(19) United States
(12) Patent Application Publication
(10) Pub. No.: US 2024/0059754 A1
(43) Pub. Date: Feb. 22, 2024

(54) CHIMERIC ANTIGEN RECEPTORS AND ENHANCEMENT OF ANTI-TUMOR ACTIVITY

(71) Applicant: iCell Gene Therapeutjics LLC, Stony Brook, NY (US)

(72) Inventors: Yupo MA, Stony Brook, NY (US); Kevin PINZ, Stony Brook, NY (US); Xun JIANG, Stony Brook, NY (US); Masayuki WADA, Stony Brook, NY (US); Kevin CHEN, Stony Brook, NY (US)

(73) Assignee: iCell Gene Therapeutjics LLC, Stony Brook, NY (US)

(21) Appl. No.: 17/590,983

(22) Filed: Feb. 2, 2022

Related U.S. Application Data
(63) Continuation of application No. 16/061,987, filed on Jun. 13, 2018, now abandoned.
(60) Provisional application No. 62/270,657, filed on Dec. 22, 2015.

(51) Int. Cl. C07K 14/705; A61K 35/17; A61K 39/00; A61P 35/02; C07K 16/28; C12N 5/0783

(52) U.S. Cl. CPC C07K 14/70517; A61K 35/17; A61K 39/0011; A61P 35/02; C07K 14/70521; C07K 14/70578; C07K 16/2896; C12N 5/0646; C07K 2319/50; C12N 2510/00; A61K 2039/5158; A61K 2039/5156; C07K 2319/00; C07K 14/5434

(57) ABSTRACT

This disclosure relates to chimeric antigen receptors targeting T cell malignancies. The present disclosure also relates to the development of methods for inactivation with engineered CARs, to enhance T cell functions or reduce T cell suppression.

### CROSS-REFERENCE TO RELATED APPLICATIONS

This application is continuation of U.S. application Ser. No. 16/061,987 filed Apr. 26, 2020, which is now abandoned, and was a national stage filing of international PCT application PCT/US2016/068358 filed Dec. 22, 2016, and claims the benefit of U.S. Provisional application Ser. No. 62/270,657, filed Dec. 22, 2015, all of which is herein incorporated by reference in its entirety.

### SEQUENCE LISTING

A Sequence Listing conforming to the rules of WIPO Standard ST.25 is hereby incorporated by reference. Said Sequence Listing has been filed as an electronic document via PatentCenter in ASCII formatted text. The electronic document, created on Jul. 3, 2023, is entitled “2541-5_PCT-US-CON_ST25.txt”, and is 109,580 bytes in size.

### FIELD OF THE INVENTION

This disclosure relates to chimeric antigen receptors targeting T cell malignancies. The present disclosure also relates to the development of methods for inactivation with engineered CARs, to enhance T cell functions or reduce T cell suppression.

### BACKGROUND

CAR therapy is a powerful new adoptive immunotherapy technique that has shown promise in recent years as a potential curative option for a number of solid and hematological cancers, most notably B-cell lymphoma (Firor, Jares et al. 2015). CAR therapy utilizes modified patient immune cells, typically T cells, but also NK cells in some cases (Arai, et al. 2008 “Infusion of the allogeneic cell line NK-92 in patients with advanced renal cell cancer or melanoma: a phase I trial.” Cytotherapy 10(6): 625-632.), to target and eliminate cancer cells in a major histocompatibility complex (MHC)-independent manner. In the CAR construct, the single-chain fragment variable (scFv) is linked to the T cell costimulatory and intracellular T cell receptor signaling domains via hinge and transmembrane regions, thus allowing for directed immune cell cytotoxic activity against a recognized surface protein. However, many immunosuppressive modifications that malignant cells make to their extracellular microenvironment as well as their own surface protein expression have thus far limited the efficacy and scope of CAR therapies for specific cancers. There is a critical need to develop a more powerful CAR by uncoupling an enhancer of the anti-tumor immune response so that CARs can retain killing ability within the immunosuppressive tumor environment.

There are numerous publications concerning CAR T cells in hematological malignancies. All focus on B-cell malignancies, myeloma and acute myeloid leukemia. Reports of targeting T cell leukemias/lymphomas with CAR are rare. Although CAR technology has been reported in the literature, the final results vary from one design to another.

The identification of a suitable surface of antigen target is particularly important for the CAR efficacy. Thus, it is very difficult to predict which CAR designs will provide the desired clinical outcome. Therefore, there remains a need for the identification of suitable surface antigens and corresponding CAR designs directed to those antigens.

### SUMMARY

The present disclosure provides chimeric antigen receptors (CARS) targeting hematologic malignancies, compositions and methods of use thereof. The disclosure also provides methods of enhancing T cell function and reduce T cell suppression.

In one embodiment, the present disclosure provides an engineered cell having a first polypeptide comprising a chimeric antigen receptor polypeptide; said chimeric antigen receptor polypeptide comprising a first antigen recognition domain, a first signal peptide, a first hinge region, a first transmembrane domain, a co-stimulatory domain, and a signaling domain; and a second polypeptide comprising a second antigen recognition domain, a second hinge region, and a second transmembrane domain, wherein the second polypeptide does not comprise a co-stimulatory domain or a signaling domain.

In another embodiment, the present disclosure provides an engineered polypeptide having a first polypeptide comprising a chimeric antigen receptor polypeptide; said chimeric antigen receptor polypeptide comprising a first antigen recognition domain, a first signal peptide, a first hinge region, a first transmembrane domain, a co-stimulatory domain, and a signaling domain; and a second polypeptide comprising a second antigen recognition domain, a second hinge region, and a second transmembrane domain, wherein the second polypeptide does not comprise a co-stimulatory domain and a signaling domain; wherein the first and second polypeptide comprise a single polypeptide molecule and comprise a high efficiency cleavage site disposed between the first polypeptide and second polypeptide.

In another embodiment, the present disclosure provides a method of reducing cancer cell proliferation or increasing cancer cell death including administering an engineered cell having a first polypeptide comprising a chimeric antigen receptor polypeptide; said chimeric antigen receptor polypeptide comprising a first antigen recognition domain, a first signal peptide, a first hinge region, a first transmembrane domain, a co-stimulatory domain, and a signaling domain; and a second polypeptide comprising a second antigen recognition domain, a second hinge region, and a second transmembrane domain, wherein the second polypeptide does not comprise a co-stimulatory domain or a signaling domain to a subject in need thereof. The second antigen recognition domain includes CD2, CD3, CD4, CD5, CD7, or CD8; and innate immune cells include at least one of CD2, CD3, CD4, CD5, CD7, or CD8 and are recruited to cancer cells.

In another embodiment, the present disclosure provides an engineered cell having a first polypeptide comprising a chimeric antigen receptor polypeptide; said chimeric antigen receptor polypeptide comprising a first antigen recognition domain, a first signal peptide, a first hinge region, a first transmembrane domain, and one of a first co-stimulatory domain and a first signaling domain; and a second polypeptide comprising a tag binding domain, a second hinge region, and a second transmembrane domain, and a second co-stimulatory domain; wherein the second polypeptide does not include a signaling domain.

In another embodiment, the present disclosure provides a method of activating or expanding engineered cells including contacting the engineered cell with a tag to provide an activated or expanded engineered cells comprising a chimeric antigen receptor polypeptide. The engineered cell includes a first polypeptide comprising a chimeric antigen receptor polypeptide; said chimeric antigen receptor polypeptide comprising a first antigen recognition domain, a first signal peptide, a first hinge region, a first transmembrane domain, and one of a first co-stimulatory domain and a first signaling domain; and a second polypeptide comprising a tag binding domain, a second hinge region, and a second transmembrane domain, and a second co-stimulatory domain; wherein the second polypeptide does not include a signaling domain.

In another embodiment, the present disclosure provides a method of identifying engineered cells having a chimeric antigen receptor polypeptide. This method includes contacting the engineered cell with a tag and identifying engineered cells having a tag. The engineered cell includes a first polypeptide including a chimeric antigen receptor polypeptide; said chimeric antigen receptor polypeptide comprising a first antigen recognition domain, a first signal peptide, a first hinge region, a first transmembrane domain, and one of a first co-stimulatory domain and a first signaling domain; and a second polypeptide comprising a tag binding domain, a second hinge region, a second transmembrane domain, and a second co-stimulatory domain; wherein the second polypeptide does not include a signaling domain.

In another embodiment, the present disclosure provides a method of isolating engineered cells including contacting the engineered cell with a tag and selecting for the engineered cells that bind the tag. The engineered cell includes a first polypeptide including a chimeric antigen receptor polypeptide; said chimeric antigen receptor polypeptide comprising a first antigen recognition domain, a first signal peptide, a first hinge region, a first transmembrane domain, and one of a first co-stimulatory domain and a first signaling domain; and a second polypeptide comprising a tag binding domain, a second hinge region, a second transmembrane domain, and a second co-stimulatory domain; wherein the second polypeptide does not include a signaling domain.

In some embodiments, the present disclosure provides a method of identifying a substance specific to human CD5, that inactivate or down-regulate CD5 in CD5 expressing cells.

In some embodiments, the present disclosure provides a method of identifying a substance specific to human CD5, that recognizes the extracellular portion of CD5 in CD5 expressing cells.

### BRIEF DESCRIPTION OF THE FIGURES

FIGS. 1A-1D. Generation of CDSCAR.

FIGS. 1A-1B. The DNA gene construct and the translated protein construct for CDSCAR, and anchored CD5 scFv antibody and a cartoon demonstrating the creation and function of CDSCAR. The DNA construct of the third generation CDSCAR construct from 5′ to 3′ reads: Leader sequence, the anti-CD5 extracellular single chain variable fragment (Anti-CD5 scFv), the hinge region, the trans-membrane region, and the three intracellular signaling domains that define this construct as a 3rd generation car; CD28, 4-1BB and CD3ζ. The DNA construct of the anchored CD5 scFv antibody is the same as the CDSCAR construct without the intracellular signaling domains, as is the translated protein product for anchored CD5 scFv antibody. The translated protein constructs contain the anti-CD5 ScFv that will bind to the CD5 target, the hinge region that allows for appropriate positioning of the anti-CD5 ScFv to allow for optimal binding position, and the trans-membrane region. The complete CDSCAR protein also contains the two co-stimulatory domains and an intracellular domain of CD3 zeta chain. This construct is considered as a 3rd generation CAR: CD28, 4-1BB, and CD3ζ.

FIG. 1C. A Western blot analysis demonstrates the CDSCAR expression in HEK293 cells. HEK293 cells which transduced with GFP (as negative control) or CDSCAR lentiviruses for 48 h were used for Western blot analysis using CD3zeta antibody to determine the expression of CDSCAR. Left lane, the GFP control HEK293 cells, with no band as expected. The right lane showing a band at about 50 kDa, the molecular weight that we expected based on the CDSCAR construct.

FIG. 1D. Flow cytometry analysis for CDSCAR expression on T cells surface for lentiviral transduced CDSCAR T cells. This analysis was performed on the double transduced CDSCAR T cells at day 8 after the second lentiviral transduction. Left: isotype control T cell population (negative control); right, transduced T cells expressing CD5 CAR showing 20.53% on T cells by flow cytometry using goat anti-mouse F(AB′)2-PE.

FIGS. 2A-2C. Study Schema of the transduction of CDSCAR T-cells.

FIG. 2A. Steps for generation of CD5 CAR T cells by single transduction.

FIG. 2B. Steps for generation of CD5 CAR T cells by double transduction.

FIG. 2C. Comparisons of single and double transductions with CD5 CAR lentviruses in the down-regulation of surface CD5 expression on the T cells.

The down-regulation of extracellular CD5 protein versus GFP T-cell control over 8 days following lentiviral transduction is analyzed (top). The single transduced CDSCAR T-cells do not show complete downregulation of CD5 from cell surface by day 8, with a maximum decrease in CD5 protein expression on day 6 (middle). In the double transduced population, we note the decrease in the absolute number of CD5+, CD3+ double positive CDSCAR T-cells over time, from 65.29% on day 0 to a complete reduction of CD5 expression on day 4 (bottom). In contrast, the GFP T-cell control maintains a CD5+, CD3+ double positive population above 95% from day 2 through day 8.

FIGS. 3A-3B. Downregulation of CD5 expression on T-cells after lentiviral transduction of anchored CD5 scFv antibody after 7 days.

FIG. 3A. Study schema for the transduction of anchored CD5 scFv lentiviruses, single transduction.

FIG. 3B. Anchored CD5 scFv down-regulates or reduces the quantity of surface CD5 expression on T cells. Flow cytometry analysis demonstrating the significant decrease in CD5 protein expression (˜32%) after single transduction of CD5 scFv after 7 day incubation. Elimination of CD5 expression is observed, but not complete after 7 days, and a follow up study is currently being completed for a double transduced anchored CD5 scFv antibody.

FIGS. 4A-4D. Generation of stable CD5-deficient CCRF-CEM and Molt-4 leukemic cells using CRISPR/Cas9 lentivirus system

FIG. 4A. Flow cytometry analysis demonstrating the downregulation of CD5 expression in CCRF-CEM T-cells with CRISPR/Cas9 KD using two different primer sequences (sequence CDSA and CDSB, middle and right columns) after puromycin selection. Wild type control is seen in the left most scatter plot. Because the CRISPR/Cas9 KD technique with primer CDSA was more successful at CD5 protein downregulation, this population (denoted by the blue circle and arrow) was selected for sorting, purification and analysis in FIG. 4B.

FIG. 4B. Flow cytometry analysis data indicating the percentage of purely sorted stable CD5 negative CCRF-CEM cells transduced using the scCD5A CRISPR/Cas9 technique. We note the >99% purity of CD45 positive, CD5 negative CCRF sgCD5A T-cells.

FIG. 4C. Flow cytometry analysis demonstrating the downregulation of CD5 expression in Molt-4 T-cells with CRISPR/Cas9 KD using two different primer sequences (sequence CDSA and CDSB, middle and right columns) after puromycin treatment. Wild type control is seen the leftmost scatter plot. Because the CRISPR/Cas9 KD technique with primer CDSA was more successful at CD5 protein downregulation, this population (denoted by the blue circle and arrow) was selected for sorting, purification and analysis in FIG. 4D.

FIG. 4D. Flow cytometry analysis data indicating the percentage of purely sorted stable CD5 negative MOLT-4 cells transduced using the scCD5A CRISPR/Cas9 technique. We note the >99% purity of CD45 positive, CD5 negative MOLT-4 sgCD5A T-cells.

FIGS. 5A-5B. CDSCAR cells effectively lyseT-ALL cell lines that express CD5, and do not lyse a T leukemic cell line that does not express CD5.

FIG. 5A. Flow cytometry analysis of T-ALL cell lines alone (left column), in co-culture with GFP T-cells (middle row) and in co-culture with CDSCAR T-cells (right row). Each cell line is seen in each row. The CD5+ cell lines in the top and middle rows (CCRF-CEM and Molt-4) with the CD5− cell line seen as the bottom row (KARPAS 299). The incubation time for all co-cultures was 24 hrs, with an effector:target cell ratio of 5:1. The cell lysis compared to GFP control was over 78% for both CD5% populations, compared to the negative value for the GFP control. CCRF-CEM, 82.7% lysis vs. GFP control. Molt-4, 78.7% lysis vs. GFP control. KARPAS 299, −8.2% lysis vs. GFP control.

FIG. 5B. This bar graph denotes the T cell lysis achieved by the CDSCAR T-cells when compared to the GFP T-cells co-culture described in FIG. 5A. The cell lysis compared to GFP control was over 78% for both CD5% populations, compared to the lower value for the GFP control. (n=2 independent experiments done in duplicate).

FIGS. 6A-6D. CDSCAR cells effectively lyse T-acute lymphoblastic leukemic cells from patient samples that express CD5.

FIG. 6A. Flow cytometry analysis of T-ALL cells alone (left column), in co-culture with GFP T-cells (middle row) and in co-culture with CDSCAR T-cells (right row). Each patient cells are given a row, and are numbered to maintain patient confidentiality. The incubation time for all co-cultures was 24 hrs, with an effector:target cell ratio of 5:1. The cell lysis compared to GFP control was over 71.3% for the T-ALL-1 compared to control. The rest of the cell lines demonstrated positive cell lysis as well, but to a lesser degree, between 33-47%. This may be related to the CD5 expression for each leukemic sample, which is discussed in FIGS. 6C and 6D. T-ALL-1, 71.3% lysis vs. GFP control. T-ALL-3, 46.1% lysis vs. GFP control. T-ALL-6, 32.6% lysis vs. GFP control. T-ALL-7, 33.4% lysis vs. GFP control.

FIG. 6B. This bar graph denotes the T cell lysis achieved by the CDSCAR T-cells when compared to the GFP T-cell co-culture described in FIG. 6A. All experiments were done in duplicate.

FIG. 6C. Flow cytometry analysis data demonstrating CD3 and CD5 expression levels for patient T cell ALL samples analyzed in FIG. 6. We observe a greater percentage of CD5 positivity for T-ALL 1 and T-ALL 3 (76.64% and 89.81%) versus T-ALL 6 and T-ALL 7 (31.31% and 48.22%). Top row is unstained and bottom row is stained by CD5 and CD3 antibodies.

FIG. 6D. Flow cytometry analysis of the levels of CD5 expression on a panel of four patient sample T-ALL cell populations. The difference of mean fluorescent intensity (MFI) was determined by flow cytometry analysis (FIG. 6C).

FIG. 7. Analysis of CDSCAR T-cell killing ability for patient T-ALL cells (T-ALL-8) in details.

Flow cytometry analysis demonstrating CDSCAR T-cell killing ability for patient's T-ALL cells. The control GFP-T cell and T-ALL-8 cell co-culture are seen on the left, and the CDSCAR co-culture with T-ALL 8 is seen on the right. We note avid lysis of all CD5 positive cells, both CD34 positive (circled in red) and CD34 negative (circled in green, T cells), with no lysis noted for CD5 negative cells. When compared to GFP control, CDSCAR T cells lyse at minimum 93.1% of T-ALL-8 cells when compared to GFP control. Experiment was done in duplicate. In addition, CDSCAR T cells essentially eliminate the T cell population (CD5+CD34−, circled in green).

### CD123CAR NK Cells

FIG. 8B. Generation of CD123CAR.

FIG. 8: CD123 CAR construct comprises a leader sequence, anti-CD123 scFv (single-chain fragment), a hinge (H) region, a transmembrane domain (TM), co-stimulatory domains (CD28, 4-1BB), and an intracellular signaling domain, CD3 zeta chain.

To identify the CD123CAR construct, transfected 293-T cells were subjected to Western blot analysis. Immunoblotting with an anti-CD3zeta monoclonal antibody showed bands of predicted size for CD123CAR. In contrast, no CD3zeta expression was shown for the GFP control vector.

FIGS. 9A-9C. Generation of NK-92 NK cells expressing CD123CAR

FIG. 9A. Steps for transduction of CD123CAR lentiviruses on NK-92 cells.

FIG. 9B. CD123CAR expression in NK-92 cells. The transduced NK-92 cells were analyzed by flow cytometry with biotinylated Goat-anti-Mouse against F(Ab)2 region, showing about 35% of NK-92 cells expressing CD123CAR.

FIG. 9C. Transduced NK-92 cells were sorted by flow cytometry after expansion. The CD123CAR-expressing cells were approximately 94% positive after sorting.

FIGS. 10A-10D. CD123CAR NK cells effectively lysed AML cell lines, KG1A and TF1.

FIG. 10A. Immunophenotype of AML cell line, KG1A showing CD123 expression

FIG. 10B. CD123CAR NK cells show a potent activity in killing KG1A cells in co-culture assay. After 4 hour co-culture with different ratios of effector CD123CAR NK cells: target cells (E:T), the cells were analyzed by flow cytometry. Three different ratios were used: 0.5 to 1, 1 to 1, and 2 to 1. The cell lysis by CD123 NK cells (upper panel) was compared to that of GFP control (lower panel). The ability of CD123CAR NK cells to lyse target CD123 cells was evaluated by comparing the amount of residual CD123+ GFP T-cells after co-culture.

FIG. 10C. CD123CARNK cells effectively kill TF1 cells. After 4 hour co-culture with different ratios of effective CD123CAR NK cells: target cells (E:T), the cells were analyzed by flow cytometry. Three different ratios were used: 0.5 to 1, 1 to 1, and 2 to 1. The cell lysis by CD123 NK cells (upper panel) was compared to that of GFP control (lower panel).

FIG. 10D. The Bar graph shows the NK cell lysis achieved by CD123CAR NK cells when compared to the GFP NK cells co-culture. The cell lysis compared to GFP control was about 38% to 50% for both AML cell lines.

FIGS. 11A-11C. CD123CAR NK-92 cells effectively kill leukemic cells from a human AML sample (AML-9).

FIG. 11A. Immunophenotype of a human AML sample (AML-9) showing the expression of both CD34 and CD123 by flow cytometry.

FIG. 11B. CD123CARNK-92 cells effectively lysed human AML cells expressing CD34 and CD123 at a ratio of 5 to 1 (E:T). After 4-hour co-culture, cells were analyzed by flow cytometry and the cell lysis was compared to that of GFP controls.

FIG. 11C. The bar graph shows the AML-9 lysis by CD123NK cells. The cell lysis compared to GFP controls was over 40% for CD123 or CD34 leukemic population.

### CD3CAR NK Cells

FIGS. 12A-12BA. Characterization of CD3CAR NK cells.

FIG. 12A. A co-culture assay showing the incubation of CD3CAR NK cells with target GFP transduced T-cells expressing the CD3 T-cell marker. The NK CAR cells are identified by the CD56 marker for NK cells and co-culture conditions were carried out in NK cell media with 2.5% serum. Co-cultures were incubated for 4 or 24 hours and labeled for flow cytometry analysis. FIG. 12AB. The ability of CD3-CAR NK cells to lyse target CD3 cells at the ratios of 2:1 and 5:1, was evaluated by comparing the amount of residual CD3+ GFP T-cells after co-culture. Human peripheral blood T cells were used as target cells to determine the potency of killing of CD3CAR NK cells on these cells. Note: The NK-92 cells were used to generate CD3CAR NK cells.

FIG. 12B. Importantly, with an increased incubation period, target CD3+ GFP T-cells were shown to be lysed with over 85% efficiency at a dosage of 5:1 effector to target cell ratio after 24 hour co-culture. Results from FIG. 12BA summarized in the bar graph. These studies indicate that CD3 CAR has a potent killing activity.

FIG. 13. A schematic showing eCAR (enhanced CAR) construct. The construct consists of a SFFV promoter driving the expression of anchored protein or marker or domains thereof, for T cells having at least one anti-CD5 extracellular single-chain variable fragment (scFv) and at least one unit of CAR linked by a peptide, such as, P2A. Upon cleavage of the linker, the eCAR splits to generate the anchored CD5 scFv protein and a CAR (s) on T cells. The CAR has its own scFv domain targeting a tumor antigen(s) and the CAR may have one or more of co-stimulatory domains.

### CD4CAR NK Cells

FIGS. 14A-14C. CD4CAR construct.

FIG. 14A. Schematic representation of recombinant lentiviral vector encoding third generation CD4CAR, driven by spleen focus-forming virus (SFFV) promoter. The construct contains a leader sequence, anti-CD4 scFv, hinge domain (H), transmembrane (TM) and signaling domains CD28, 4-1BB, and CD3 zeta.

FIG. 14B. HEK293FT cells were transfected with GFP (lane 1) and CD4CAR (lane 2) lentiviral plasmids. Forty-eight hours after transfection, cells were removed and subsequently used for Western blot analysis with mouse anti-human CD3z antibody.

FIG. 14C. Illustration of third-generation CAR NK cells targeting CD4 expressing cells.

FIGS. 15A-15C. CD4CAR NK cell production.

FIG. 15A. Experimental design of NK cell activation, transduction, and expansion.

FIG. 15B. CD4CAR expression levels on NK cells prior to being sorted by FACS (N=3); NK cells were incubated with biotin-labeled goat anti-mouse F(Ab′)2 followed by streptavidin-PE. (C) CD4CAR expression on NK cells after sorting and expansion, prior to co-culture experiments (N=3); NK cells were again incubated with biotin-labeled goat anti-mouse F(Ab′)2 followed by streptavidin-PE. NK cells were then transduced with either GFP or CDCAR lentiviral supernatant, or cultured for non-transduced control. After 7 days of incubation, cells were harvested and analyzed by flow cytometry with Biotin-labeled goat anti-mouse F(Ab′)2 followed by streptavidin-PE. NK cells were greater than 85% positive for CD4CAR after following FACS sorting for CD4CAR^{high}. Isotype control of sorted cells on the left.

FIG. 15C. Data after sorting; 85% positive for CAR; isotype control of sorted cells on the left.

FIGS. 16A-16L. CD4 CAR NK cells ablate CD4 positive leukemia and lymphoma cells in co-culture assays. All direct flow data shown in 16A-16F were from co-cultures performed at an effector to target ratio of 2:1 for 24 hours, after which, cells were stained with mouse anti-human CD56 and CD4 antibodies. Each assay consists of activated NK cells transduced with either GFP (center) or CD4CAR (right) lentiviral supernatant and incubated with target cells, as well as target cells incubated alone as a control (left). CD4CAR NK cells eliminated Karpas 299 leukemic T-cells (A, B; N=3), HL-60 T-cells (C, D; N=2), and CCRF-CEM cells (E, F; N=2). CD4CAR NK cells eliminated primary T-cell leukemia cells from patients with CD4 expressing T-cell leukemia/Sézary syndrome (16G and 16H; N=2) and CD4 expressing pediatric T-cell ALL (16I and 16J; N=2). Co-culture, 24 hours—CD4CAR NK vs Cord blood T cells (16K). (16L) Bar graph summarizing co-culture assay results for both 2:1 and 5:1 ratios. In CD4 positive cell lines only, CD4CAR NK cells showed enhanced killing ability relative to that of GFP NK cells. All co-cultures were done for 24 hours.

FIG. 17. Co-culture killing curve. CD4CAR NK cells kill CD4-expressing leukemic cell lines in a dose dependent manner. NK cells transduced with either CD4CAR or GFP control lentiviral supernatant were incubated with CFSE-stained Karpas 299 cells or CMTMR-stained CCRF-CEM cells at 1:4, 1:2, and 1:1 effector to target ratios. After 24 hours, 7-AAD dye was added and remaining live cells were analyzed by flow cytometry. Percent killing of target cells was measured by comparing CD4 positive Karpas 299 or CCRF-CEM cell survival in CD4CAR NK cell co-cultures relative to that in control GFP NK cell co-cultures.

FIG. 18. CD4CAR NK cells were incubated at co-culture effector:target ratios of 2:1 and 5:1 respectively with 500 CD34+CB cells for 24 hours in NK cell media supplemented with IL-2. Experimental controls used were CD34+ cells alone, and non-transduced NK cells co-cultured at respective 2:1 and 5:1 effector:target ratios with CD34+CB cells. Hematopoietic compartment output was assessed via formation of erythroid burst-forming units (BFU-E) and number of granulocyte/monocyte colony-forming units (CFU-GM) at Day 16. CFU statistical analysis was performed via 2-way ANOVA with alpha set at 0.05.

FIGS. 19A-19E. CD4CAR NK cells demonstrate anti-leukemic effects in vivo. NSG mice were sublethally irradiated and intradermally injected with luciferase-expressing Karpas 299 cells (Day 0) to induce measurable tumor formation. On day 1 and every 5 days for a total of 6 courses, mice were intravenously injected with 5×10 6 CD4CAR NK cells or GFP NK control cells.

FIGS. 19A and B. On days 7, 14, and 21, mice were injected subcutaneously with RediJect D-Luciferin™ and subjected to IVIS imaging.

FIG. 19C. Average light intensity measured for the CD4CAR NK injected mice was compared to that of GFP NK injected mice.

FIG. 19D. On day 1, and every other day after, tumor size area was measured and the average tumor size between the two groups was compared.

FIG. 19E. Percent survival of mice was measured and compared between the two groups.

FIGS. 20A-20B. CDSCAR NK-92 cells almost eliminate CD5 positive cells in a co-culture assay.

FIG. 20A. Flow cytometry analysis of GFP transduced T cells alone, in co-culture with non-transduced NK-92 cells, in co-culture with two sorted preparations of CDSCAR NK-92 cells-(a) and CDSCAR-NK92 cells-(b) (from left to right).

The NK-92 cells were transduced with lenti-CDSCAR viruses. The transduced cells, CDSNK-92 cells were sorted by flow cytometry. The CD5 positive T cells were labeled with GFP with lenti-GFP viruses. The incubation time for all co-cultures was 4 hrs, with an effector:target cell ratio of 4:1. GFP-transduced T-cells were detected by CD3-PerCP antibody and NK-92 cells were identified CD56-PE antibody. The % of cell lysis compared to non-transduced NK92 (control), both of sorted CD5CARNK-92 cells showed over 88% of cell lysis activity against T cells.

FIG. 20B. The results from 20A were summarized in the bar graph. This bar graph indicates % of cell lysis activity by sorted CDSCAR NK-92-(a) or -(b) cells compared to the non-transduced NK92 cells in co-culture assay described in above.

FIG. 21. Depicts an embodiment of an engineered cell of the present disclosure. In particular, the cell surface of an engineered cell is shown with a first polypeptide comprising a chimeric antigen receptor polypeptide; said chimeric antigen receptor polypeptide comprising a first antigen recognition domain, a first signal peptide, a first hinge region, a first transmembrane domain, a co-stimulatory domain, and a signaling domain; and a second polypeptide comprising a second antigen recognition domain, a second signal peptide, a second hinge region, and a second transmembrane domain, wherein the second polypeptide does not comprise a co-stimulatory domain or a signaling domain.

FIG. 22. Depicts an embodiment of an engineered cell of the present disclosure. In particular, the cell surface of an engineered cell is shown with a first polypeptide comprising a chimeric antigen receptor polypeptide; said chimeric antigen receptor polypeptide comprising a first antigen recognition domain, a first signal peptide, a first hinge region, a first transmembrane domain, and one of a first co-stimulatory domain and a first signaling domain; and a second polypeptide comprising a tag binding domain, a second signal peptide, a second hinge region, and a second transmembrane domain, and a second co-stimulatory domain; wherein the second polypeptide does not comprise a signaling domain.

### DETAILED DESCRIPTION

A chimeric antigen receptor (CAR) polypeptide includes a signal peptide, an antigen recognition domain, a hinge region, a transmembrane domain, at least one co-stimulatory domain, and a signaling domain.

First-generation CARs include CD3z as an intracellular signaling domain, whereas second-generation CARs include at least one single co-stimulatory domain derived from various proteins. Examples of co-stimulatory domains include, but are not limited to, CD28, CD2, 4-1BB (CD137, also referred to as “4-BB”), and OX-40 (CD124). Third generation CARs include two co-stimulatory domains, such as, without limiting, CD28, 4-1BB, CD134 (OX-40), CD2, and/or CD137 (4-1BB).

As used herein, the terms “peptide,” “polypeptide,” and “protein” are used interchangeably, and refer to a compound having amino acid residues covalently linked by peptide bonds. A protein or peptide must contain at least two amino acids, and no limitation is placed on the maximum number of amino acids that can include a protein's or peptide's sequence. Polypeptides include any peptide or protein having two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A “signal peptide” includes a peptide sequence that directs the transport and localization of the peptide and any attached polypeptide within a cell, e.g. to a certain cell organelle (such as the endoplasmic reticulum) and/or the cell surface.

The signal peptide is a peptide of any secreted or transmembrane protein that directs the transport of the polypeptide of the disclosure to the cell membrane and cell surface, and provides correct localization of the polypeptide of the present disclosure. In particular, the signal peptide of the present disclosure directs the polypeptide of the present disclosure to the cellular membrane, wherein the extracellular portion of the polypeptide is displayed on the cell surface, the transmembrane portion spans the plasma membrane, and the active domain is in the cytoplasmic portion, or interior of the cell.

In one embodiment, the signal peptide is cleaved after passage through the endoplasmic reticulum (ER), i.e. is a cleavable signal peptide. In an embodiment, the signal peptide is human protein of type I, II, III, or IV. In an embodiment, the signal peptide includes an immunoglobulin heavy chain signal peptide.

The “antigen recognition domain” includes a polypeptide that is selective for or targets an antigen, receptor, peptide ligand, or protein ligand of the target; or a polypeptide of the target.

The antigen recognition domain may be obtained from any of the wide variety of extracellular domains or secreted proteins associated with ligand binding and/or signal transduction. The antigen recognition domain may include a portion of Ig heavy chain linked with a portion of Ig light chain, constituting a single chain fragment variable (scFv) that binds specifically to a target antigen. The antibody may be monoclonal or polyclonal antibody or may be of any type that binds specifically to the target antigen. In another embodiment, the antigen recognition domain can be a receptor or ligand. In particular embodiments, the target antigen is specific for a specific disease condition and the disease condition may be of any kind as long as it has a cell surface antigen, which may be recognized by at least one of the chimeric receptor construct present in the compound CAR architecture. In a specific embodiment, the chimeric receptor may be for any cancer for which a specific monoclonal or polyclonal antibody exists or is capable of being generated. In particular, cancers such as neuroblastoma, small cell lung cancer, melanoma, ovarian cancer, renal cell carcinoma, colon cancer, Hodgkin's lymphoma, and childhood acute lymphoblastic leukemia have antigens specific for the chimeric receptors.

In some embodiments, antigen recognition domain can be non-antibody protein scaffolds, such as but not limited to, centyrins, non-antibody protein scaffolds that can be engineered to bind a variety of specific targets with high affinity. Centyrins are scaffold proteins based on human consensus tenascin FN3 domain, are usually smaller than scFv molecules.

The target specific antigen recognition domain preferably includes an antigen binding domain derived from an antibody against an antigen of the target, or a peptide binding an antigen of the target, or a peptide or protein binding an antibody that binds an antigen of the target, or a peptide or protein ligand (including but not limited to a growth factor, a cytokine, or a hormone) binding a receptor on the target, or a domain derived from a receptor (including but not limited to a growth factor receptor, a cytokine receptor or a hormone receptor) binding a peptide or protein ligand on the target.

In one embodiment, the antigen recognition domain includes the binding portion or variable region of a monoclonal or polyclonal antibody directed against (selective for) the target.

In another embodiment, the antigen recognition domain includes Camelid single domain antibody, or portions thereof. In one embodiment, Camelid single-domain antibodies include heavy-chain antibodies found in camelids, or VHH antibody. A VHH antibody of camelid (for example camel, dromedary, llama, and alpaca) refers to a variable fragment of a camelid single-chain antibody (See Nguyen et al, 2001; Muyldermans, 2001), and also includes an isolated VHH antibody of camelid, a recombinant VHH antibody of camelid, or a synthetic VHH antibody of camelid.

In another embodiment, the antigen recognition domain includes ligands that engage their cognate receptor. By way of example, APRIL is a ligand that binds the TAC1 receptor or the BCMA receptor. In accordance with subject matter disclosed herein, the antigen recognition domain includes APRIL, or a fragment thereof. By way of further example, BAFF is a ligand that binds the BAFF-R receptor or the BCMA receptor. In accordance with the subject matter disclosed herein, the antigen recognition domain includes BAFF, or a fragment thereof. In another embodiment, the antigen recognition domain is humanized.

It is understood that the antigen recognition domain may include some variability within its sequence and still be selective for the targets disclosed herein. Therefore, it is contemplated that the polypeptide of the antigen recognition domain may be at least 95%, at least 90%, at least 80%, or at least 70% identical to the antigen recognition domain polypeptide disclosed herein and still be selective for the targets described herein and be within the scope of the disclosure.

The target includes interleukin 6 receptor, NY-ESO-1, alpha fetoprotein (AFP), glypican-3 (GPC3), BCMA, BAFF-R, TACI, LeY, CD13, CD14, CD15, CD30, BAFF, TACI, CD19, CD20, CD22, CD33, CD41, CD61, CD64, CD68, CD117, CD123, CD138, CD267, CD269, CD38, Flt3 receptor, CS1, CD45, ROR1, PSMA, MAGE A3, Glycolipid, glypican 3, F77, GD-2, WT1, CEA, HER-2/neu, MAGE-3, MAGE-4, MAGE-5, MAGE-6, alpha-fetoprotein, CA 19-9, CA 72-4, NY-ESO, FAP, ErbB, c-Met, MART-1, CD30, EGFRvIII, immunoglobin kappa and lambda, CD38, CD52, CD3, CD4, CD8, CD5, CD7, CD2, and CD138

In another embodiment, the target includes any portion interleukin 6 receptor, NY-E50-1, alpha fetoprotein (AFP), glypican-3 (GPC3), BCMA, BAFF-R, TACI, LeY, CD13, CD14, CD15, CD30, BAFF, TACI, CD19, CD20, CD22, CD33, CD41, CD61, CD64, CD68, CD117, CD123, CD138, CD267, CD269, CD38, Flt3 receptor, CS1, CD45, TACI, ROR1, PSMA, MAGE A3, Glycolipid, glypican 3, F77, GD-2, WT1, CEA, HER-2/neu, MAGE-3, MAGE-4, MAGE-5, MAGE-6, alpha-fetoprotein, CA 19-9, CA 72-4, NY-ESO, FAP, ErbB, c-Met, MART-1, CD30, EGFRvIII, immunoglobin kappa and lambda, CD38, CD52, CD3, CD4, CD8, CD5, CD7, CD2, and CD138.

In one embodiment, the target includes surface exposed portions of interleukin 6 receptor, NY-ESO-1, alpha fetoprotein (AFP), glypican-3 (GPC3), BCMA, BAFF-R, TACI, LeY, CD13, CD14, CD15, CD30, BAFF, TACI, CD19, CD20, CD22, CD33, CD41, CD61, CD64, CD68, CD117, CD123, CD138, CD267, CD269, CD38, Flt3 receptor, CS1, CD45, TACI, ROR1, PSMA, MAGE A3, Glycolipid, glypican 3, F77, GD-2, WT1, CEA, HER-2/neu, MAGE-3, MAGE-4, MAGE-5, MAGE-6, alpha-fetoprotein, CA 19-9, CA 72-4, NY-ESO, FAP, ErbB, c-Met, MART-1, CD30, EGFRvIII, immunoglobin kappa and lambda, CD38, CD52, CD3, CD4, CD8, CD5, CD7, CD2, and CD138 polypeptides.

In another embodiment, the target antigens include viral or fungal antigens, such as E6 and E7 from the human papillomavirus (HPV) or EBV (Epstein Barr virus) antigens; portions thereof; or surface exposed regions thereof.

The hinge region is a sequence positioned between for example, including, but not limited to, the chimeric antigen receptor, and at least one co-stimulatory domain and a signaling domain. The hinge sequence may be obtained including, for example, from any suitable sequence from any genus, including human or a part thereof. Such hinge regions are known in the art. In one embodiment, the hinge region includes the hinge region of a human protein including CD-8 alpha, CD28, 4-1BB, OX40, CD3-zeta, T cell receptor a or β chain, a CD3 zeta chain, CD28, CD3ε, CD45, CD4, CD5, CD8, CD8a, CD9, CD16, CD22, CD33, CD37, CD64, CD80, CD86, CD134, CD137, ICOS, CD154, functional derivatives thereof, and combinations thereof.

In one embodiment the hinge region includes the CD8 a hinge region.

In some embodiments, the hinge region includes one selected from, but is not limited to, immunoglobulin (e.g. IgG1, IgG2, IgG3, IgG4, and IgD).

The transmembrane domain includes a hydrophobic polypeptide that spans the cellular membrane. In particular, the transmembrane domain spans from one side of a cell membrane (extracellular) through to the other side of the cell membrane (intracellular or cytoplasmic).

The transmembrane domain may be in the form of an alpha helix or a beta barrel, or combinations thereof. The transmembrane domain may include a polytopic protein, which has many transmembrane segments, each alpha-helical, beta sheets, or combinations thereof.

In one embodiment, the transmembrane domain that is naturally associated with one of the domains in the CAR is used. In another embodiment, the transmembrane domain is selected or modified by amino acid substitution to avoid binding of such domains to the transmembrane domains of the same or different surface membrane proteins to minimize interactions with other members of the receptor complex.

For example, a transmembrane domain includes a transmembrane domain of a T-cell receptor α or β chain, a CD3 zeta chain, CD28, CD3ε, CD45, CD4, CD5, CD7, CD8, CD9, CD16, CD22, CD33, CD37, CD64, CD80, CD86, CD68, CD134, CD137, ICOS, CD41, CD154, functional derivatives thereof, and combinations thereof.

In one embodiment, the transmembrane domain is artificially designed so that more than 25%, more than 50% or more than 75% of the amino acid residues of the domain are hydrophobic residues such as leucine and valine. In one embodiment, a triplet of phenylalanine, tryptophan and valine is found at each end of the synthetic transmembrane domain.

In one embodiment, the transmembrane domain is the CD8 transmembrane domain. In another embodiment, the transmembrane domain is the CD28 transmembrane domain. Such transmembrane domains are known in the art.

The signaling domain and co-stimulatory domain include polypeptides that provide activation of an immune cell to stimulate or activate at least some aspect of the immune cell signaling pathway.

In an embodiment, the signaling domain includes the polypeptide of a functional signaling domain of CD3 zeta, common FcR gamma (FCER1G), Fc gamma Rlla, FcR beta (Fc Epsilon Rib), CD3 gamma, CD3 delta, CD3 epsilon, CD79a, CD79b, DNAX-activating protein 10 (DAP10), DNAX-activating protein 12 (DAP12), active fragments thereof, functional derivatives thereof, and combinations thereof. Such signaling domains are known in the art.

In an embodiment, the CAR polypeptide further includes one or more co-stimulatory domains. In an embodiment, the co-stimulatory domain is a functional signaling domain from a protein including OX40; CD27; CD28; CD30; CD40; PD-1; CD2; CD7; CD258; Natural killer Group 2 member C (NKG2C); Natural killer Group 2 member D (NKG2D), B7-H3; a ligand that binds to at least one of CD83, ICAM-1, LFA-1 (CD1 la/CD18), ICOS, and 4-1BB (CD137); CDS; ICAM-1; LFA-1 (CD1a/CD18); CD40; CD27; CD7; B7-H3; NKG2C; PD-1; ICOS; active fragments thereof; functional derivatives thereof; and combinations thereof.

As used herein, the at least one co-stimulatory domain and signaling domain may be collectively referred to as the intracellular domain. As used herein, the hinge region and the antigen recognition may be collectively referred to as the extracellular domain.

The present disclosure further provides a polynucleotide encoding the chimeric antigen receptor polypeptide described herein.

The term “polynucleotide” as used herein is defined as a chain of nucleotides. Polynucleotide includes DNA and RNA. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and polymerase chain reaction (PCR), and the like, and by synthetic means.

The polynucleotide encoding the CAR is easily prepared from an amino acid sequence of the specified CAR by any conventional method. A base sequence encoding an amino acid sequence can be obtained from the aforementioned NCBI RefSeq IDs or accession numbers of GenBenk for an amino acid sequence of each domain, and the nucleic acid of the present disclosure can be prepared using a standard molecular biological and/or chemical procedure. For example, based on the base sequence, a polynucleotide can be synthesized, and the polynucleotide of the present disclosure can be prepared by combining DNA fragments which are obtained from a cDNA library using a polymerase chain reaction (PCR).

In one embodiment, the polynucleotide disclosed herein is part of a gene, or an expression or cloning cassette.

The polynucleotide described above can be cloned into a vector. A “vector” is a composition of matter which includes an isolated polynucleotide and which can be used to deliver the isolated polynucleotide to the interior of a cell. Numerous vectors are known in the art including, but not limited to, linear polynucleotides, polynucleotides associated with ionic or amphiphilic compounds, plasmids, phagemid, cosmid, and viruses. Viruses include phages, phage derivatives. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like. In one embodiment, vectors include cloning vectors, expression vectors, replication vectors, probe generation vectors, integration vectors, and sequencing vectors.

In an embodiment, the vector is a viral vector. In an embodiment, the viral vector is a retroviral vector or a lentiviral vector. In an embodiment, the engineered cell is virally transduced to express the polynucleotide sequence.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the patient either in vivo or ex vivo. A number of retroviral systems are known in the art. In some embodiments, adenovirus vectors are used. A number of adenovirus vectors are known in the art. In one embodiment, lentivirus vectors are used.

Viral vector technology is well known in the art and is described, for example, in Sambrook et al, (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endomiclease sites, and one or more selectable markers, (e.g., WO 01/96584; WO 01/29058; and U.S. Pat. No. 6,326,193).

Lentiviral vectors have been well known for their capability of transferring genes into human T cells with high efficiency but expression of the vector-encoded genes is dependent on the internal promoter that drives their expression. A strong promoter is particularly important for the third or fourth generation of CARs that bear additional co-stimulatory domains or genes encoding proliferative cytokines as increased CAR body size does not guarantee equal levels of expression. There are a wide range of promoters with different strength and cell-type specificity. Gene therapies using CAR T cells rely on the ability of T cells to express adequate CAR body and maintain expression over a long period of time. The EF-1α promoter has been commonly selected for the CAR expression.

The present disclosure relates to an expression vector containing a strong promoter for high level gene expression in T cells or NK cells. In further embodiment, the inventor discloses a strong promoter useful for high level expression of CARs in T cells or NK cells. In particular embodiments, a strong promoter relates to the SFFV promoter, which is selectively introduced in an expression vector to obtain high levels of expression and maintain expression over a long period of time in T cells or NK cells. Expressed genes prefer CARs, T cell co-stimulatory factors and cytokines used for immunotherapy.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1 a (EF-1 a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the disclosure should not be limited to the use of constitutive promoters, inducible promoters are also contemplated as part of the disclosure. The use of an inducible promoter provides a molecular switch capable of turning on expression of the polynucleotide sequence, which is operatively linked when such expression is desired, or turning off the expression when expression is not desired. Examples of inducible promoters include, but are not limited to a metalothionine promoter, a glucocorticoid promoter, a progesterone promoter, and a tetracycline promoter.

Expression of chimeric antigen receptor polynucleotide may be achieved using, for example, expression vectors including, but not limited to, at least one of a SFFV (spleen-focus forming virus) or human elongation factor 11a (EF) promoter, CAG (chicken beta-actin promoter with CMV enhancer) promoter human elongation factor 1a (EF) promoter. Examples of less-strong/lower-expressing promoters utilized may include, but is not limited to, the simian virus 40 (SV40) early promoter, cytomegalovirus (CMV) immediate-early promoter, Ubiquitin C (UBC) promoter, and the phosphoglycerate kinase 1 (PGK) promoter, or a part thereof. Inducible expression of chimeric antigen receptor may be achieved using, for example, a tetracycline responsive promoter, including, but not limited to, TRE3GV (Tet-response element, including all generations and preferably, the 3rd generation), inducible promoter (Clontech Laboratories, Mountain View, CA) or a part or a combination thereof.

In a preferred embodiment, the promoter is an SFFV promoter or a derivative thereof. It has been unexpectedly discovered that SFFV promoter provides stronger expression and greater persistence in the transduced cells in accordance with the present disclosure.

“Expression vector” refers to a vector including a recombinant polynucleotide comprising expression control sequences operatively linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide. The expression vector may be a bicistronic or multicistronic expression vectors. Bicistronic or multicistronic expression vectors may include (1) multiple promoters fused to each of the open reading frames; (2) insertion of splicing signals between genes; fusion of genes whose expressions are driven by a single promoter; (3) insertion of proteolytic cleavage sites between genes (self-cleavage peptide); and (iv) insertion of internal ribosomal entry sites (IRESs) between genes.

In one embodiment, the disclosure provides an engineered cell having at least one chimeric antigen receptor polypeptide or polynucleotide.

An “engineered cell” means any cell of any organism that is modified, transformed, or manipulated by addition or modification of a gene, a DNA or RNA sequence, or protein or polypeptide. Isolated cells, host cells, and genetically engineered cells of the present disclosure include isolated immune cells, such as NK cells and T cells that contain the DNA or RNA sequences encoding a chimeric antigen receptor or chimeric antigen receptor complex and express the chimeric receptor on the cell surface. Isolated host cells and engineered cells may be used, for example, for enhancing an NK cell activity or a T lymphocyte activity, treatment of cancer, and treatment of infectious diseases.

In an embodiment, the engineered cell includes immunoregulatory cells. Immunoregulatory cells include T-cells, such as CD4 T-cells (Helper T-cells), CD8 T-cells (Cytotoxic T-cells, CTLs), and memory T cells or memory stem cell T cells. In another embodiment, T-cells include Natural Killer T-cells (NK T-cells).

In an embodiment, the engineered cell includes Natural Killer cells. Natural killer cells are well known in the art. In one embodiment, natural killer cells include cell lines, such as NK-92 cells. Further examples of NK cell lines include NKG, YT, NK-YS, HANK-1, YTS cells, and NKL cells.

NK cells mediate anti-tumor effects without the risk of GvHD and are short-lived relative to T-cells. Accordingly, NK cells would be exhausted shortly after destroying cancer cells, decreasing the need for an inducible suicide gene on CAR constructs that would ablate the modified cells.

In accordance with the present disclosure, it was surprisingly found that NK cells provide a readily available cell to be engineered to contain and express the chimeric antigen receptor polypeptides disclosed herein.

Allogeneic or autologous NK cells induce a rapid immune response but disappear relatively rapidly from the circulation due to their limited lifespan. Thus, applicants surprisingly discovered that there is reduced concern of persisting side effects using CAR cell based therapy.

According to one aspect of the present disclosure, NK cells can be expanded and transfected with CAR polynucleotides in accordance to the present disclosure. NK cells can be derived from cord blood, peripheral blood, iPS cells and embryonic stem cells. According to one aspect of the present disclosure, NK-92 cells may be expanded and transfected with a CAR. NK-92 is a continuously growing cell line that has features and characteristics of natural killer (NK) cells (Arai, Meagher et al. 2008). NK-92 cell line is IL-2 dependent and has been proven to be safe(Arai, Meagher et al. 2008) and feasible. CAR expressing NK-92 cells can be expanded in the serum free-medium with or without co-culturing with feeder cells. A pure population of NK-92 carrying the CAR of interest may be obtained by sorting.

In one embodiment, engineered cells include allogeneic T cells obtained from donors that are modified to inactivate components of TCR (T cell receptor) involved in MHC recognition. As a result, TCR deficient T cells would not cause graft versus host disease (GVHD).

In some embodiments, the engineered cell may be modified to prevent expression of cell surface antigens. For example, an engineered cell may be genetically modified to delete the native CD5 gene to prevent expression and cell surface display thereof.

In some embodiments, the engineered cell includes an inducible suicide gene (“safety switch”) or a combination of safety switches, which may be assembled on a vector, such as, without limiting, a retroviral vector, lentiviral vector, adenoviral vector or plasmid. Introduction of a “safety switch” greatly increases safety profile and limits on-target or off-tumor toxicities of the compound CARs. The “safety switch” may be an inducible suicide gene, such as, without limiting, caspase 9 gene, thymidine kinase, cytosine deaminase (CD) or cytochrome P450. Other safety switches for elimination of unwanted modified T cells involve expression of CD20 or CD19 or truncated epidermal growth factor receptor in T cells. All possible safety switches are have been contemplated and are embodied in the present disclosure.

In some embodiments, the suicide gene is integrated into the engineered cell genome.

### Multiple Polypeptide Units

The present disclosure provides an engineered cell having at least two distinct polypeptide units. A polypeptide unit, as used herein includes a polypeptide that has an antigen binding domain, or tag binding domain, a signal peptide, a hinge region, and a transmembrane domain. Two polypeptides are distinct if they have different antigen binding domains, different tag binding domains, or an antigen binding domain and a tag binding domain.

In some embodiments, multiple units of polypeptides are expressed in a T or NK cell using bicistronic or multicistronic expression vectors. There are several strategies can be employed to construct bicistronic or multicistronic vectors including, but not limited to, (1) multiple promoters fused to the polypeptide open reading frames; (2) insertion of splicing signals between polypeptide units; fusion of polypeptides whose expressions are driven by a single promoter; (3) insertion of proteolytic cleavage sites between polypeptide units (self-cleavage peptide); and (iv) insertion of internal ribosomal entry sites (IRESs).

In a preferred embodiment, multiple polypeptide units are expressed in a single open reading frame (ORF), thereby creating a single polypeptide having multiple polypeptide units. In this embodiment, an amino acid sequence or linker containing a high efficiency cleavage site is disposed between each polypeptide unit.

As used herein, high cleavage efficiency is defined as more than 50%, more than 70%, more than 80%, or more than 90% of the translated protein is cleaved. Cleavage efficiency may be measured by Western Blot analysis, as described by Kim 2011.

Furthermore, in a preferred embodiment, there are equal amounts of cleavage product, as shown on a Western Blot analysis.

Examples of high efficiency cleavage sites include porcine teschovirus-1 2A (P2A), FMDV 2A (abbreviated herein as F2A); equine rhinitis A virus (ERAV) 2A (E2A); Thoseaasigna virus 2A (T2A); cytoplasmic polyhedrosis virus 2A (BmCPV2A); flacherie Virus 2A (BmIFV2A); or a combination thereof. In a preferred embodiment, the high efficiency cleavage site is P2A. High efficiency cleavage sites are described in Kim J H, Lee S-R, Li L-H, Park H-J, Park J-H, Lee K Y, et al. (2011) High Cleavage Efficiency of a 2A Peptide Derived from Porcine Teschovirus-1 in Human Cell Lines, Zebrafish and Mice. PLoS ONE 6(4): e18556, the contents of which are incorporated herein by reference.

In embodiments wherein multiple CAR units are expressed in a single open reading frame (ORF), expression is under the control of a strong promoter. Examples of strong promoters include the SFFV promoter, and derivatives thereof.

In another embodiment, the hinge region is designed to exclude amino acids that may cause undesired intra- or intermolecular interactions. For example, the hinge region may be designed to exclude or minimize cysteine residues to prevent formation of disulfide bonds. In another embodiment, the hinge region may be designed to exclude or minimize hydrophobic residues to prevent unwanted hydrophobic interactions.

### Engineered Cell Having CAR Polypeptide and Accessory Component

In another embodiment, the present disclosure provides an engineered cell having at least one chimeric antigen receptor polypeptide and an accessory component.

In one embodiment, the present disclosure provides an engineered cell having at least two distinct chimeric antigen receptor polypeptides and an accessory component.

As used herein, an accessory component includes a biological molecule that promotes or enhances the activity of the engineered cell having the chimeric antigen receptor polypeptide. Accessory component include cytokines. In another embodiment, accessory component includes IL-2, IL-7, IL-12, IL-15, IL-21, PD-1, PD-L1, CSF1R, CTAL-4, TIM-3, and TGFR beta, receptors for the same, and functional fragments thereof.

Accessory components may be expressed by the engineered cell described herein and displayed on the surface of the engineered cell or the accessory component may be secreted into the surrounding extracellular space by the engineered cell. Methods of surface display and secretion are well known in the art. For example, the accessory component may be a fusion protein with a peptide that provides surface display or secretion into the extracellular space.

The effect of the accessory component may be complemented by additional factors such as accessory component receptors and functional fragments thereof. The additional factors may be co-expressed with the accessory component as a fusion protein or expressed as a separate peptide and secreted into the extracellular space.

In one embodiment, the accessory component is IL-15. In this instance, the additional factor is the IL-15 receptor, and functional fragments thereof. Functional fragments include the IL-15 receptor, IL-15RA, and the sushi domain of IL-15RA.

Interleukin (IL)-15 and its specific receptor chain, IL-15Ra (IL-15-RA) play a key functional role in various effector cells, including NK and CD8 T cells. CD8+ T cells can be modified to express autocrine growth factors including, but not limited to, IL-2, 11-7, IL21, or IL-15, to sustain survival following transfer in vivo. Without wishing to be bound by theory, it is believed that IL-15 could overcome the CD4 deficiency to induce primary and recall memory CD8T cells. Overexpression of IL-15-RA or an IL-15 IL-RA fusion on CD8 T cells significantly enhances its survival and proliferation in-vitro and in-vivo. In some embodiments, CD4CAR or any CAR can include expressing any one or more of moieties, IL-15, IL15RA and IL-15/IL-15R or IL15-RA/IL-15, or a part or a combination thereof, to enhance survival or proliferation of CAR T or NK, and to improve expansion of memory CAR CD8+ T cells.

The present disclosure relates to an engineered cell having a CAR as described herein and any one or more of moieties of IL-15, IL15RA and IL-15/IL-15R or IL15-RA/IL-15, or a part or a combination thereof, to enhance survival or persistent or proliferation of CAR T or NK for treating cancer in a patient.

### Methods of Generating Engineered Cells

Any of the polynucleotides disclosed herein may be introduced into an engineered cell by any method known in the art.

In one embodiment, CAR polynucleotides are delivered to the engineered cell by any viral vector as disclosed herein.

In one embodiment, to achieve enhanced safety profile or therapeutic index, any of the engineered cells disclosed herein can be constructed as a transient RNA-modified “biodegradable” version or derivatives, or a combination thereof. The RNA-modified CARs of the present disclosure may be electroporated into T cells or NK cells.

In some embodiments of the present disclosure, any of the engineered cells disclosed herein may be constructed in a transponson system (also called a “Sleeping Beauty”), which integrates the CAR DNA into the host genome without a viral vector.

### Engineered Cell Having CAR Polypeptide and Accessory Component

In another embodiment, the present disclosure provides a method making an engineered cell that expresses at least one CAR unit and an accessory component.

In some embodiments, at least one CAR unit and accessory component is expressed in a T or NK cell using bicistronic or multicistronic expression vectors. There are several strategies can be employed to construct bicistronic or multicistronic vectors including, but not limited to, (1) multiple promoters fused to the CARs' open reading frames; (2) insertion of splicing signals between units of CAR; fusion of CARs whose expressions are driven by a single promoter; (3) insertion of proteolytic cleavage sites between units of CAR (self-cleavage peptide); and (iv) insertion of internal ribosomal entry sites (IRESs).

In a preferred embodiment, at least one CAR unit and an accessory component are expressed in a single open reading frame (ORF), thereby creating a single polypeptide having at least one CAR unit and an accessory component. In this embodiment, an amino acid sequence or linker containing a high efficiency cleavage site is disposed between each CAR unit and between a CAR unit and accessory component. In this embodiment, the ORF is under the control of a strong promoter. Examples of strong promoters include the SFFV promoter, and derivatives thereof.

Furthermore, in a preferred embodiment, there are equal amounts of cleavage product, as shown on a Western Blot analysis.

### Methods of Treatment Using the Compositions Disclosed Herein

The compositions and methods of this disclosure can be used to generate a population of T lymphocyte or NK cells that deliver both primary and co-stimulatory signals for use in immunotherapy in the treatment of cancer, in particular, the treatment of lung cancer, melanoma, breast cancer, prostate cancer, colon cancer, renal cell carcinoma, ovarian cancer, brain cancer, sarcoma, leukemia, and lymphoma.

Immunotherapeutics generally rely on the use of immune effector cells and molecules to target and destroy cancer cells. The effector may be a lymphocyte carrying a surface molecule that interacts, either directly or indirectly, with a tumor cell target. Various effector cells include cytotoxic T cells, NK cells, and NK-92 cells. The compositions and methods described in the present disclosure may be utilized in conjunction with other types of therapy for cancer, such as chemotherapy, surgery, radiation, gene therapy, and so forth. The compositions and methods described in the present disclosure may be utilized in other disease conditions that rely on immune responses such as inflammation, immune diseases, and infectious diseases.

In accordance with the present disclosure, natural killer (NK) cells represent alternative cytotoxic effectors for CAR driven killing. Unlike T-cells, NK cells do not need pre-activation and constitutively exhibit cytolytic functions.

Further, NK cells are known to mediate anti-cancer effects without the risk of inducing graft-versus-host disease (GvHD).

There is aberrant overexpression of CD123 on CD34+CD38− AML cells, while the normal bone marrow counterpart CD34+CD38− does not express CD123. This population of CD123+, CD34+CD38− has been considered as LSCs as these cells are able to initiate and maintain the leukemic process into immunodeficient mice.

The number of CD34+/CD38−/CD123+LSCs can be used to predict the clinical outcome for AML patients. The CD34+/CD38−/CD123+ cells, greater than 15% in AML patients, are associated with a lack of complete remission and unfavorable cytogenetic profiles. In addition, the presence of more than 1% of CD34+/CD38-/CD123+ cells could also have a negative impact on disease-free survival and overall survival.

Identification of appropriate surface target antigens is a prerequisite for developing CAR T/NK cells in adaptive immune therapy.

CD123, the alpha chain of the interleukin 3 receptor, is overexpressed on a variety of hematologic malignancies, including acute myeloid leukemia (AML), B-cell acute lymphoblastic leukemia (B-ALL), hairy cell leukemia, and blastic plasmocytoid dendritic neoplasms. CD123 is absent or minimally expressed on normal hematopoietic stem cells. More importantly, CD123 is expressed on a subset of leukemic cells related to leukemic stem cells (LSCs), the ablation of which is essential in preventing disease refractoriness and relapse.

CD33 is a transmembrane receptor expressed on 90% of malignant cells in acute myeloid leukemia. Thus, according to the present disclosure, CD123 and CD33 target antigens are particularly attractive from a safety standpoint.

Multiple myeloma (MM) is a haematological malignancy with a clonal expansion of plasma cells. Despite important advances in the treatment, myeloma remains an incurable disease; thus novel therapeutic approaches are urgently needed.

CS1 (also called as CD319 or SLAMF7) is a protein encoded by the SLAMF7 gene. The surface antigen CS1 is a robust marker for normal plasma cells and myeloma cells (malignant plasma cells).

Tumour necrosis factor receptor superfamily, member 17 (TNFRSF17), also referred to as B-cell maturation antigen (BCMA) or CD269 is almost exclusively expressed at the terminal stages of plasma cells and malignant plasma cells. Its expression is absent other tissues, indicating the potential as a target for CAR T or NK cells.

Malignant plasma cells display variable degrees of antigenic heterogeneity for CD269 and CS1. A single CAR unit product targeting either CD269 or CS1 could target the majority of the cells in a bulk tumor resulting in an initial robust anti-tumor response. Subsequently residual rare non-targeted cells are expanded and cause a disease relapse. While multiple myeloma is particularly heterogeneous, this phenomena could certainty apply to other leukemias or tumors. A recent clinical trial at NIH using BCMA CAR T cells showed a promising result with a complete response in some patients with multiple myeloma. However, these patients relapsed after 17 weeks, which may be due to the antigen escape. The antigen escape is also seen in CD19 CAR and NY-ESO1 CAR T cell treatments. Thus, there is an urgent need for more effective CAR T cell treatment in order to prevent the relapse.

In one aspect of the present disclosure, BCMA and CS1 are the targets for BCMACS1 CAR therapy.

BAFF (B-cell-activation factor) and APRIL (a proliferation-induced ligand) are two TNF homologs that bind specifically TACI (also called as TNFRSF1 3B or CD267) and BCMA with high affinity. BAFF (also known as BLyS) binds BAFF-R and functionally involves in the enhancement of survival and proliferation of later stage of B cells. BAFF has been shown to involve some autoimmune disorders. APRIL plays an important role in the enhancement of antibody class switching. Both BAFF and APRIL have been implicated as growth and survival factors for malignant plasma cells.

In further embodiments, the target antigens can include at least one of this group, but not limited to, ROR1, PSMA, MAGE A3, Glycolipid, glypican 3, F77, GD-2, WT1, CEA, HER-2/neu, MAGE-3, MAGE-4, MAGE-5, MAGE-6, alpha-fetoprotein, CA 19-9, CA 72-4, NY-ESO, FAP, ErbB, c-Met, MART-1, CD30, EGFRvIII, immunoglobin kappa and lambda, CD5, CD38, CD52, CD3, CD4, CD8, CD5, CD7, CD2, and CD138. The target antigens can also include viral or fungal antigens, such as E6 and E7 from the human papillomavirus (HPV) or EBV (Epstein Barr virus) antigens.

In some embodiments, the CAR polypeptide is part of an expressing gene or a cassette. In a preferred embodiment, the expressing gene or the cassette may include an accessory gene or a tag or a part thereof, in addition to the CDSCAR. The accessory gene may be an inducible suicide gene or a part thereof, including, but not limited to, caspase 9 gene, thymidine kinase, cytosine deaminase (CD) or cytochrome P450. The “suicide gene” ablation approach improves safety of the gene therapy and kills cells only when activated by a specific compound or a molecule. In some embodiments, the suicide gene is inducible and is activated using a specific chemical inducer of dimerization (CID).

In some embodiments, safety switch can include the accessory tags are a c-myc tag, CD20, CD52 (Campath), truncated EGFR gene (EGFRt) or a part or a combination thereof. The accessory tag may be used as a nonimmunogenic selection tool or for tracking markers.

In some embodiments, safety switch can include a 24-residue peptide that corresponds to residues 254-277 of the RSV F glycoprotein A2 strain (NSELLSLINDMPITNDQKKLMSNN) (SEQ ID NO: 27).

In some embodiments, safety switch can include the amino acid sequence of TNF a bound by monoclonal anti-TNF a drugs.

Administration of any of the engineered cells described herein may be supplemented with the co-administration of a CAR enhancing agent. Examples of CAR enhancing agents include immunomodulatory drugs that enhance CAR activities, such as, but not limited to agents that target immune-checkpoint pathways, inhibitors of colony stimulating factor-1 receptor (CSF1R) for better therapeutic outcomes. Agents that target immune-checkpoint pathways include small molecules, proteins, or antibodies that bind inhibitory immune receptors CTLA-4, PD-I, and PD-1-1, and result in CTLA.-4 and PD-1/PD-L1 blockades. As used herein, enhancing agent includes accessory component as described above.

### Anchor

In one embodiment, the disclosure provides an engineered cell having a first polypeptide including a chimeric antigen receptor polypeptide; said chimeric antigen receptor polypeptide comprising a first antigen recognition domain, a first signal peptide, a first hinge region, a first transmembrane domain, a co-stimulatory domain, and a signaling domain; and a second polypeptide comprising a second antigen recognition domain, a second signal peptide, a second hinge region, and a second transmembrane domain, wherein the second polypeptide does not comprise a co-stimulatory domain or a signaling domain.

In one embodiment, the first antigen recognition domain is CD123 and the second antigen recognition domain is CD3. See SEQ ID NO: 9 and SEQ ID NO: 10.

In one embodiment, the first antigen recognition domain is CD123 and the second antigen recognition domain is CD33. See SEQ ID NO: 11 and SEQ ID NO: 12.

In one embodiment, the first antigen recognition domain is CD123 and the second antigen recognition domain is CD269. See SEQ ID NO: 13 and SEQ ID NO: 14.

In one embodiment, the first antigen recognition domain is CD123 and the second antigen recognition domain is CD20. See SEQ ID NO: 15 and SEQ ID NO: 16.

In one embodiment, the first antigen recognition domain is CD123 and the second antigen recognition domain is CD20b. See SEQ ID NO: 17 and SEQ ID NO: 18.

In one embodiment, the first antigen recognition domain is CD123 and the second antigen recognition domain is CD22. See SEQ ID NO: 19 and SEQ ID NO: 20.

In one embodiment, the first antigen recognition domain is CD123 and the second antigen recognition domain is CD19b. See SEQ ID NO: 21 and SEQ ID NO: 22.

In one embodiment, the antigen recognition domain and the second antigen recognition domain are specific and bind to different targets.

In one embodiment, the first and second transmembrane domain are different.

In one embodiment, the disclosure provides an engineered cell having a first polypeptide including a chimeric antigen receptor polypeptide; said chimeric antigen receptor polypeptide comprising a first antigen recognition domain, a first signal peptide, a first hinge region, a first transmembrane domain, a co-stimulatory domain, and a signaling domain; a second polypeptide comprising a second antigen recognition domain, a second signal peptide, a second hinge region, and a second transmembrane domain; and a third polypeptide comprising a third antigen recognition domain, a third signal peptide, a third hinge region, and a third transmembrane domain. The second polypeptide and third polypeptide do not have a co-stimulatory domain or a signaling domain.

In one embodiment, the disclosure provides an engineered cell having a first polypeptide including a chimeric antigen receptor polypeptide; said chimeric antigen receptor polypeptide comprising a first antigen recognition domain, a first signal peptide, a first hinge region, a first transmembrane domain, a co-stimulatory domain, and a signaling domain; and at least one polypeptide unit wherein each polypeptide unit includes a antigen recognition domain, a signal peptide, a hinge region, and a transmembrane region. Wherein each of the polypeptide units do not have a co-stimulatory domain or a signaling domain.

### Anchor Redirector

In one embodiment, the engineered cell may be used to recruit innate immune cells to a target of the engineered cell by virtue of the second polypeptide. For example, an engineered cell having a CDX antigen recognition domain will target the engineered cell to cells having the CDX antigen. Furthermore, by virtue of the tag binding domain a tag will be bound as well. When the tag binding domain is CD2 or CD7, innate or host natural killer cells and t-cells are bound and recruited to the target of the first polypeptide. When the tag binding domain is CD4, CD3, CD5, or CD8, then T-cells are bound and recruited to the target of the first polypeptide.

### Identifying Engineered Cells

In one embodiment, the disclosure provides a method of identifying engineered cell. In this embodiment, the engineered cell includes a first polypeptide including a chimeric antigen receptor polypeptide; said chimeric antigen receptor polypeptide comprising a first antigen recognition domain, a first signal peptide, a first hinge region, a first transmembrane domain, a co-stimulatory domain, and a signaling domain; and a second polypeptide comprising a second antigen recognition domain, a second signal peptide, a second hinge region, and a second transmembrane domain, wherein the second polypeptide does not comprise a co-stimulatory domain or a signaling domain.

The engineered cell is contacted with a tag that binds the tag binding domain. Upon binding, the tag may be identified by any known method. Examples of identification may plate reader or with flow cytometry.

In one embodiment, engineered cells having the polypeptides described above may be identified among a population of cells by flow cytometry.

### Isolating Engineered Cells

In one embodiment, the present disclosure provides a method of isolating engineered cells having a chimeric antigen receptor polypeptide. In this embodiment, the engineered cell includes a first polypeptide including a chimeric antigen receptor polypeptide; said chimeric antigen receptor polypeptide comprising a first antigen recognition domain, a first signal peptide, a first hinge region, a first transmembrane domain, a co-stimulatory domain, and a signaling domain; and a second polypeptide comprising a second antigen recognition domain, a second signal peptide, a second hinge region, and a second transmembrane domain, wherein the second polypeptide does not comprise a co-stimulatory domain or a signaling domain.

The engineered cell is contacted with a tag that binds the tag binding domain. Upon binding, the engineered cell may be isolated by virtue of the tag bound to the tag binding domain. The engineered cell may be isolated from a population of engineered cells and non-engineered cells.

For example, a population of engineered cells described above and non-engineered cells is contacted with a tag. The tag is bound to a solid support. The engineered cells bind the solid support by virtue of interaction between the tag binding domain and the tag.

In one embodiment, the second peptide of the engineered cell described above includes a strep tag polypeptide as the tag binding domain. These cells are incubated with streptactin beads. The mixture is then loaded onto a column and washed with buffer. The engineered cells are predominantly bound to the streptactin beads in the column. The cells can be eluted from the column by incubating the engineered cells bound to the streptactin beads with biotin or a biotin derivative.

The eluted cells are enriched for the engineered cells described above.

### Anchor Tag Enhancer

In one embodiment, the present disclosure provides an engineered cell having a first polypeptide comprising a chimeric antigen receptor polypeptide; said chimeric antigen receptor polypeptide comprising a first antigen recognition domain, a first signal peptide, a first hinge region, a first transmembrane domain, and one of a first co-stimulatory domain and a first signaling domain; and a second polypeptide comprising a tag binding domain, a second signal peptide, a second hinge region, and a second transmembrane domain, and a second co-stimulatory domain; wherein the second polypeptide does not comprise a signaling domain.

In one embodiment, the first and second transmembrane domains are different.

In one embodiment, the first and second signal peptides are different.

Tag binding domain is a polypeptide sequence that is specific for and binds a tag. The tag binding domain may be an antibody, binding portion or variable region of a monoclonal antibody, or scFv.

Examples of tag binding domain includes AviTag, a peptide allowing biotinylation by the enzyme BirA and so the protein can be isolated by streptavidin (GLNDIFEAQKIEWHE) (SEQ ID NO: 28); Calmodulin-tag, a peptide bound by the protein calmodulin (KRRWKKNFIAVSAANRFKKISSSGAL) (SEQ ID NO: 29); polyglutamate tag, a peptide binding efficiently to anion-exchange resin such as Mono-Q (EEEEEE) (SEQ ID NO: 30); E-tag, a peptide recognized by an antibody (GAPVPYPDPLEPR) (SEQ ID NO: 31); FLAG-tag, a peptide recognized by an antibody (DYKDDDDK) (SEQ ID NO: 32); HA-tag, a peptide from hemagglutinin recognized by an antibody (YPYDVPDYA) (SEQ ID NO: 331; His-tag, 5-10 histidines bound by a nickel or cobalt chelate (HHHHHH) (SEQ ID NO: 34); Myc-tag, a peptide derived from c-myc recognized by an antibody (EQKLISEEDL) (SEQ ID NO: 35); NE-tag, a novel 18-amino-acid synthetic peptide (TKENPRSNQEESYDDNES) (SEQ ID NO: 36) recognized by a monoclonal IgG1 antibody, which is useful in a wide spectrum of applications including Western blotting, ELISA, flow cytometry, immunocytochemistry, immunoprecipitation, and affinity purification of recombinant proteins; S-tag, a peptide recognized by an antibody (KETAAAKFERQHMDS) (SEQ ID NO: 37); SBP-tag, a peptide which binds to streptavidin (MDEKTTGWRGGHVVEGLAGELEQLRARLEHHPQGQREP) (SEQ ID NO: 38); Softag 1, for mammalian expression (SLAELLNAGLGGS) (SEQ ID NO: 39); Softag 3, for prokaryotic expression (TQDPSRVG) (SEQ ID NO: 40); Strep-tag, a peptide which binds to streptavidin or the modified streptavidin called streptactin (Strep-tag II: WSHPQFEK) (SEQ ID NO: 41); TC tag, a tetracysteine tag that is recognized by FlAsH and ReAsH biarsenical compounds (CCPGCC) (SEQ ID NO: 42); V5 tag, a peptide recognized by an antibody (GKPIPNPLLGLDST) (SEQ ID NO: 43); VSV-tag, a peptide recognized by an antibody (YTDIEMNRLGK) (SEQ ID NO: 44); Xpress tag, which binds ProBond resin (DLYDDDDK) (SEQ ID NO: 45).

The tag is a molecule that is specific for and binds the tag binding domain. The tag may be an antibody, streptavidin, biotin, a metal chelate, HIS, MYC, HA, agarose, V5, Maltose, GST, S-protein, or GFP.

In another embodiment, the tag may include a detectable moiety. The tag may be a detectable moiety itself or it may be bound or conjugated to a detectable moiety. Examples of detectable moieties include GFP, fluorescent marker or dye, APC, PE, pacific blue, or FITC.

Examples of fluorescent markers or dyes include Alexa Fluor™ dyes, coumarin, rhodamine, xanthene (such as fluorescein), and cyanine dyes.

In one embodiment, the tag binding domain is FLAG and T cell co-stimulatory domain is CD28. See SEQ ID NO: 23 and SEQ ID NO: 24.

In one embodiment, the tag binding domain is FLAG and T cell co-stimulatory domain is 4-1BB. See SEQ ID NO: 25 and SEQ ID NO: 26.

In one embodiment, the tag is conjugated to an antibody.

### Activating or Expanding Engineered Cells

In one embodiment, the present disclosure provides a method of activating or expanding engineered cells having a chimeric antigen receptor polypeptide. In this embodiment, the engineered cell includes a first polypeptide a first polypeptide having a chimeric antigen receptor polypeptide; said chimeric antigen receptor polypeptide comprising a first antigen recognition domain, a first signal peptide, a first hinge region, a first transmembrane domain, and one of a first co-stimulatory domain and a first signaling domain; and a second polypeptide comprising a tag binding domain, a second signal peptide, a second hinge region, and a second transmembrane domain, and a second co-stimulatory domain; wherein the second polypeptide does not comprise a signaling domain.

The engineered cells may be activated or expanded in vivo or ex vivo.

In one embodiment, the first polypeptide includes a 4-1BB costimulation domain and a CD3 zeta signaling domain; and the second polypeptide includes a CD28 costimulation domain. Binding of the first antigen receptor polypeptide to its target results in CD3 zeta signaling and 4-1BB costimulation. Upon binding of the tag to the binding domain of the second polypeptide, CD28 of the second polypeptide provides further costimulation. Therefore, binding of the first antigen recognition domain and tag binding domain to their cognate targets provides controllable activation or expansion of the engineered cell.

### Identifying Engineered Cells

In one embodiment, the present disclosure provides a method of identifying engineered cells having a chimeric antigen receptor polypeptide. In this embodiment, the engineered cell includes a first polypeptide a first polypeptide having a chimeric antigen receptor polypeptide; said chimeric antigen receptor polypeptide comprising a first antigen recognition domain, a first signal peptide, a first hinge region, a first transmembrane domain, and one of a first co-stimulatory domain and a first signaling domain; and a second polypeptide comprising a tag binding domain, a second signal peptide, a second hinge region, and a second transmembrane domain, and a second co-stimulatory domain; wherein the second polypeptide does not comprise a signaling domain.

The engineered cell is contacted with a tag that binds the tag binding domain. Upon binding, the tag may be identified by any known method. Examples of identification methods include by way of plate reader or flow cytometry.

In one embodiment, engineered cells having the polypeptides described above may be identified among a population of cells by flow cytometry.

### Isolating Engineered Cells

In one embodiment, the present disclosure provides a method of isolating engineered cells having a chimeric antigen receptor polypeptide. In this embodiment, the engineered cell includes a first polypeptide a first polypeptide having a chimeric antigen receptor polypeptide; said chimeric antigen receptor polypeptide comprising a first antigen recognition domain, a first signal peptide, a first hinge region, a first transmembrane domain, and one of a first co-stimulatory domain and a first signaling domain; and a second polypeptide comprising a tag binding domain, a second signal peptide, a second hinge region, and a second transmembrane domain, and a second co-stimulatory domain; wherein the second polypeptide does not comprise a signaling domain.

The engineered cell is contacted with a tag that binds the tag binding domain. Upon binding, the engineered cell may be isolated by virtue of the tag bound to the tag binding domain. The engineered cell may be isolated from a population of engineered cells and non-engineered cells.

For example, a population of engineered cells described above and non-engineered cells is contacted with a tag. The tag is bound to a solid support. The engineered cells bind the solid support by virtue of interaction between the tag binding domain and the tag.

In one embodiment, the second peptide of the engineered cell described above includes a strep tag polypeptide as the tag binding domain. These cells are incubated with streptactin beads. The mixture is then loaded onto a column and washed with buffer. The engineered cells are predominantly bound to the streptactin beads in the column. The cells can be eluted from the column by incubating the engineered cells bound to the streptactin beads with biotin or a biotin derivative.

The eluted cells are enriched for the engineered cells described above.

Deletion of Engineered Cells with an Anchor Tag In Vivo

In one embodiment, an engineered cell expressing an anchor tag protein can be contact with non-endogenous binding molecules and induce cell death. In a further embodiment, an engineered cell with an anchor tag can be marked by tagged antibodies, which lead to cell death. The system can be used as a “safety switch” for CAR T/NK cell therapy.

### CD5 Deficient Engineered Cells

In one embodiment, the engineered cells disclosed herein are CD5 deficient. An engineered cell is CD5 deficient when it has reduced level of cell surface CD5 as compared to a wild type CD5 immune cell. As used herein, “deficient” is used interchangeably with “down-regulated” or “inactivated”. Methods of down-regulation or inactivation are commonly known in the art.

The CD5 deficient engineered cell may have more than 10% reduction, more than 25% reduction, more than 50% reduction, more than 75% reduction, more than 90% reduction, more than 95% reduction in the level of cell surface CD5 as compared to a wild type CD5 immune cell.

CD5 deficient engineered cells may be made by genetic editing methods such as CRISPR based methods.

In one embodiment, a CD5 deficient engineered cell may be made by expression of a polypeptide having a CD5 antigen recognition domain. The antigen recognition domain may be an antibody, binding portion or variable region of a monoclonal antibody, or scFv. In one embodiment, the CD5 antigen recognition domain may be part of a chimeric antigen receptor polypeptide.

### CD5 Target for CARs

T cells are a type of white blood cells that are critical for the immune system and represent the key of adaptive immunity. T cells can function as “soldiers” that search out and destroy the targeted infectious agents and cancer cells. Natural killer cells (NK cells) are a type of cytotoxic lymphocyte critical to the innate immune system and provide rapid responses to infectious agents or tumor formation.

CD5 is expressed in more than 80% of T cell acute lymphoblastic leukemia (-T-ALL). A one-treatment option is to treat patients with anti-CD5 antibodies as T-cell leukemias/lymphomas expressing the CD5 surface molecule. However attempts have met limited success.

Selection of an appropriate target antigen is a key consideration for CAR therapy. For better outcomes, target for T cell therapy should be universally expressed on the cancer cells to be eliminated, allowing for effective tumor lysis while avoiding relapse. In addition, target antigen expression should be restricted to the cancer cell type to avoid off tumor, on target effects. CD5 (Lyt-1) is a 67 kDa type-1 membrane glycoprotein belonging to the scavenger receptor cysteine-rich (SRCR) superfamily. The CD5 gene, located on chromosome 11, encodes a surface receptor consisting of an extracellular region made up of three tandem SRCR domains (D1, D2, and D3), a transmembrane region, and an intracytoplasmic signaling domain. CD5 is universally expressed on T-cells, and is expressed on a small subset of B cells (B-1a cells) that include B regulatory/suppressor cells (Bregs), a main source of the immunosuppressive interleukin Il-10.

Importantly, CD5 expression is restricted to the hematologic compartment, limiting the possibility of off-tumor, on-target effects in non-hematopoietic tissues. CD5 dysfunction is associated with a number of autoimmune diseases, and a decreased ability of the immune system to recognize and eliminate malignant cells that upregulate CD5 expression on tumor infiltrating lymphocytes (TILs). For this reason, CD5 may be an important target in autoimmune disorders and malignant diseases, including Systemic Lupus Erythmatosis, rheumatoid arthritis, Insulin-Dependent Diabetes Mellitus, Autoimmune Nephropathy, EBV-associated hemophagocytic lymphoshistocytosis, and malignant hematologic disease.

Anti-CD5 directed monoclonal antibody therapy has been used to treat rheumatoid arthritis in Phase I and II studies with only transient treatment-associated adverse effects that quickly resolved without sequelae. In addition, the modulation of CD5 utilizing monoclonal antibody therapy has been utilized in human subjects as a safe and effective prophylaxis for graft-versus-host disease following bone marrow transplantation therapy (BMT therapy), although the therapeutic benefit was offset by graft rejection and loss of the graft versus leukemia effect. Since CD5 expression and function is relegated to the hematologic compartment, modification of CD5 expression in treatment of autoimmune disorders and malignancies is a potentially powerful tool that can increase the potency of immunotherapies including CAR therapy.

The present disclosure provides the CDSCAR constructs under the control of a high expression promoter. CD5CARs contain an anti-CD5 scFv targeting an epitope on CD5. In some embodiment, the CARs including scFv fused to the co-stimulatory domain (s) and intracellular signaling domain, CD3 zeta chain via a hinge region and a transmembrane domain.

In some embodiments, CDSCAR includes at least one or more than one of co-stimulatory domains selected from but is not limited to, CD28, CD2, 4-1BB (CD137, also referred to as “4-BB”), OX-40 (CD124), CDS, ICAM-1, LFA-1 (CD1 la/CD18), CD40, CD27, CD7, B7-H3, NKG2C, PD-1, and ICOS.

In some embodiments, CDSCAR includes one hinge region selected from, but is not limited to, CD8a, CD4, immunoglobulin (e.g. IgG1, IgG2, IgG3, IgG4 and IgD).

In some embodiments, CDSCAR includes at least one of transmembrane domains selected from, but is not limited to, CD28, CD4, CD5, CD7, CD3 epsilon, CD8, CD9, CD16, CD22, CD33, CD137, CD154, CD86, CD41, CD64, and CD68. The transmembrane domain can be a polypeptide, derivative or analogue comprising predominantly hydrophobic residues.

Expression of CDSCAR could be controlled by a promoter selected from, but is not limited to, spleen focus-forming virus (SFFV) or human elongation factor 11a (EF) promoter, CAG (chicken beta-actin promoter with CMV enhancer) promoter human elongation factor 1α (EF) promoter, the simian virus 40 (SV40) early promoter, cytomegalovirus (CMV) immediate-early promoter, Ubiquitin C (UBC) promoter, and the phosphoglycerate kinase 1 (PGK) promoter. The constructs of the present disclosure may use a tetracycline responsive promoter, TRE3GV (Tet-response element, including all generations and preferably, the 3rd generation) (Clontech Laboratories, Mountain View, CA).

The disclosure includes a method of generating CDSCAR. In some embodiments, the CDSCAR is generated using T-cells. In other embodiments, the CD5 is using primary NK cells isolated from the peripheral blood or cord blood, and NK-92 cells. The development of an NK CAR construct could bypass the need for HLA matching, such that CAR NK cells can be administered “off-the-shelf” to any mammal with a disease or cancer.

In some embodiments, the extracellular domain may be derived from any of the wide variety of extracellular domains or receptors or secreted proteins associated with ligand binding and/or signal transduction. The extracellular domain may include scFv derived from a portion of Ig heavy chain linked with a portion of Ig light chain. In further embodiments, the scFv is derived from the CD5 monoclonal antibody, polyclonal antibodies or other means of antibody technology.

In some embodiments, the vectors for expressing the CDSCAR can be viral expression vectors including, but are not limited to, lentivirus-, retrovirus-, or adenovirus-based vectors.

In some embodiments, CDSCAR may combine with an inducible suicide gene as a “safety switch”. The “safety switch” may be an inducible suicide gene, but is not limited to, caspase 9 gene, thymidine kinase, cytosine deaminase (CD) or cytochrome P450. Other safety switches for elimination of unwanted modified CAR T cells involve expression of CD20 or CD19 or truncated epidermal growth factor receptor in T cells.

In other embodiments, CD5CARs may be constructed as a transient RNA-modified “biodegradable derivatives”. The RNA-modified derivatives may be electroporated into a T or NK cell. In a further embodiment, CDSCAR may be constructed in a transponson system also called a “Sleeping Beauty”), which integrates the compound CAR DNA into the host genome without a viral vector.

In some embodiments, CDSCAR may be constructed as a second-generation CAR, which bears at least one single co-stimulatory domain. CDSCAR may also be constructed as a third-generation CAR, which comprises two co-stimulatory domains.

In further embodiments, CDSCAR T or NK cells can be designed with inducible or constitutive cytokine (s) to accumulate and maintain therapeutic levels of cytokines in the target tissue. The cytokines can include, but is not limited to, interleukin 12, interleukin 7, and interleukin 15.

In some embodiments, the CDSCAR of the present disclosure may act as a bridge to bone marrow transplant for those patients who are not longer responding to chemotherapy or have minimal residual diseases and are not eligible for bone marrow transplant. In further embodiments, CDSCAR can eliminate CD5 positive leukemic cells followed by bone marrow stem rescues to support lymphopenia.

In particular embodiments, the disclosure provides a CDSCAR engineered T cell or NK cell that targets cells that express CD5. Target cells may be, but is not limited to cancer cells, such as T cell lymphoma or leukemia, precursor acute T cell lymphoblastic leukemia/lymphoma (T-ALL), B cell chronic lymphocytic leukemia/small lymphocytic lymphoma, mantle cell lymphoma and thymic carcinoma.

In one embodiment, CDSCAR may be used for treating non-hematologic disorders including, but not limited to, rheumatoid arthritis, graft-versus-host-disease and autoimmune diseases.

In some embodiments, the CDSCAR T or T cells are co-administrated with immunomodulatory drugs, such as, but not limited to CTLA-4 and PD-1/PD-L1 blockades, or cytokines, such as IL-2 and IL12 or inhibitors of colony stimulating factor-1 receptor (CSF1R), such as FPA008, which lead to better therapeutic outcomes.

In some embodiments, the disclosure provides a CD5 CAR engineered cell that co-expresses a transgene and releases a transgenic product, such as IL-12 and IL-15 in the targeted tumor lesion and further modulate the tumor microenvironment. In a further embodiment, CDSCAR T/NK cells are administrated to a mammal as a part of cancer or any other disease treatment plan.

In one embodiment, CDSCAR T cells are derived from allogeneic lymphocytes, which are used for allogeneic infusion. In another embodiment, CDSCAR T cells are derived from autologous lymphocytes, which are used for autologous infusion. The T cells used for the generation of CDSCAR T cells are isolated from one of sources, but are not limited to the following sources: the peripheral blood, bone marrow and/or cord blood.

In one embodiment, NK cells used for the generation of CDSCAR are isolated from one of sources including, but is not limited to the peripheral blood, bone marrow, cord blood and/or derivatives from stem cells.

### CD5-Deficient T or CAR T Cells

CAR constructs have not definitively addressed the mechanisms by which the tumor evades the immune response. Activation or “priming” of the patient immune system may augment adoptive immunotherapy. Such combinatorial strategies typically seek to target checkpoint pathways in addition to the CAR regimen. Notably, nivolumab, an anti-PD-1 antibody, and ipilimumab, an anti-CTLA4 antibody, have been both been used in patients with metastatic melanoma.

CD5 is another TCR (T cell receptor) inhibitory molecule in addition to CTLA-4, TIM-3 and PD1. CD5 functions as a negative modulator of antigen-driven activation of T cells (and certain B cells as well). CD5 plays an important role in the regulation of T-cell immune responses and involves a key event in the maintenance of immune homeostasis and tolerance.

CD5 deficient mice exhibit a delayed tumor growth as compared with their wild-type counterparts. Mice with the absence of CD5 show a strong antitumor immune response that is associated with extensive tumor infiltrating by hyper-activated tumor specific T cells. The absence of CD5 expression is considered to reduce the T-cell activation threshold resulting in the enhancement of tumor-specific T-cell responses. Furthermore, CD5 expression renders tumor-infiltrating lymphocytes responsive to the specific tumor antigen stimulation.

Immunotherapies such as current T-cell based therapy or the CAR T cell approach combining strategies of the inactivation or down-regulation of CD5 expression may constitute a powerful alternative for the design of CAR T cells capable of inducing effective and prolonged antitumor responses.

The modulation of CD5 expression or activities in CAR T cells or tumor-specific T lymphocytes may significantly improve the clinical outcome of adoptive cancer immunotherapies. However, the strategies of how to inactivate CD5 or down-regulate the CD5 expression, and/or down-modulate the CD5 signaling in a preclinical setting have not been studied to date.

A transgenic mouse line expressing the extracellular domain (surface portion) of CD5 as a soluble protein, has shown that this surface protein is able to interact with undefined CD5 ligand(s) acting as a decoy receptor. These transgenic mice exhibit a lower threshold of antigen activation and an increased response against different types of antigens. In addition, these mice exhibit a significant increase of antitumor responses in non-orthotopic cancer models. Similar functional changes are also seen in the wild-type mice when administrated with exogenous recombinant CD4 surface protein. Therefore, CD5 is an important immunomodulatory target for cancer or autoimmune disease treatments.

The present disclosure relates to isolated T cells comprising inactivated or downregulated CD5 for use in immunotherapy. The present disclosure also relates to decoying CD5 receptor using the soluble CD5 surface protein, or domains or fragments thereof or CD5 antibody. In a preferred embodiment, the modified T cells are used as a therapeutic product. In a further embodiment, the modified T cells may be CAR T cells or tumor infiltrating lymphocytes (TILs).

In some embodiments, the present disclosure relates to a method for treating cancers, infections, autoimmune disorders or any other disease condition by administering modified T cells.

### Inactivated or Down-Regulated CD5 in T Cells or CAR T Cells

One of the strategies of enhancing therapeutic anti-tumor immunity is to inactivate or down-regulate negative modulators of the immune response, such as but is not limited to, CD5, PD1, and CTLA-4. The counterbalancing of stimulatory or inhibitory molecules modulates anti-tumor immune response.

CD5 has an important role in regulating T cell responses. Immunological based strategies can be performed to modulate the immune system through the manipulation of the CD5 surface expression or CD5 signaling pathway. One such immunological based approach is to use immunoglobulins targeting CD5 that block its inhibitory signals or reduce the quantity of the surface CD5 protein expression, which may enhance T cell or CAR T cell responses to infections or cancers.

The blocking CD5 inhibitory signaling with an anti-CD5 antagonist antibody can be used in conjunction with CAR T cells to enhance their responses to infections or cancers, and to prevent from T or CAR T cell exhaustion.

Therefore, the present disclosure relates to a method of immunotherapy, comprising genetically modifying T cells by inactivating or down-regulating CD5. In a particular embodiment, the method comprises the following steps:
(1) Isolating T cells from a blood or bone marrow sample, for example from one a patient or from umbilical cord blood using any protocols disclosed herein.
(2) Activating isolated T cells with anti-CD3 and IL-2 or anti-CD3/CD28 beads. Under such a condition, T cells are activated and expanded.
(3) Inactivating or down-regulating CD5. The methods of inactivation may include, but is not limited to, engineering CRISPR/Cas9 system, zinc finger nuclease (ZFNs) and TALE nucleases (TALENs) and meganucleases for inactivation of CD5. (4) Transducing said T cells with CD5 CAR or any version of CARs, thus redirecting T cells against a surface antigen expressed in malignant cells.

Malignant cells may include but are not limited to, lung cancer, melanoma, breast cancer, prostate cancer, colon cancer, renal cell carcinoma, ovarian cancer, brain cancer, sarcoma, leukemia and lymphoma neuroblastoma, small cell lung cancer, melanoma, ovarian cancer, renal cell carcinoma, colon cancer, lymphoma, childhood acute lymphoblastic leukemia, T cell acute lymphoblastic leukemia, blood cancer, T cell lymphoma, T cell leukemia, precursor acute T cell lymphoblastic leukemia, precursor acute T cell lymphoblastic lymphoma, mantle cell lymphoma, acute myeloid leukemia (AML), B-cell acute lymphoblastic leukemia (B-ALL), hairy cell leukemia, blastic plasmocytoid dendritic neoplasm, EBV-positive T-cell lymphoproliferative disorders, adult T-cell leukemia, adult T-cell lymphoma, mycosis fungoides, sezary syndrome, primary cutaneous CD30 positive T-cell lymphoproliferative disorders, peripheral T-cell lymphoma, angioimmunoblastic T-cell lymphoma, anaplastic large cell lymphoma, and thymic carcinoma.

CD5 is also expressed in CAR T cells, which offset their ability of targeting these antigens. Self-killing might occur in T cells armed with CARs targeting CD5 antigen. Therefore, it may be necessary to inactivate an endogenous CD5 antigen in a T cell when used as a target to arm CARs.

The introduction of CARs can be fulfilled before or after the inactivation of CD5 by expanding in vitro engineered T cells prior to administration to a patient. The engineered or modified T cells may be expanded in the presence of IL-2 or/and both IL-7 and IL-15, or using other molecules.

In particular embodiments, the step (3) described above can be achieved by one of the following means:
(1) Expressing anti-CD5 scFv on T cell surface linked to a transmembrane domain via a hinge region. This may result in the conversion of CD5-positive T cells to CD5 negative T cells.
(2) Expressing anti-CD5 scFv that specifically binds to CD5 protein or negative modulators of CD5 thereof, or fragments or domains thereof.

In some embodiments, a scFv (single-chain antibody) against CD5 is derived from a monoclonal or polyclonal antibody binding to intracellular CD5 and blocks the transport of CD5 protein to the cell surface. In a preferred embodiment, anti-CD5 scFv bears an ER (endoplasmic reticulum) retention sequence, KDEL (SEQ ID NO: 46). When it is expressed intracellularly and retained to the ER or Golgi, the anti-CD5 scFv entraps CD5 within the secretion pathway, which results in the prevention of CD5 proper cell surface location in a T cell.

In some embodiments, the negative modulators of CD5 may comprise any negative modulator of CD5 expression or functions. The negative modulators may be involved at different regulatory levels: such as but is not limited to, the transcriptional, post-transcriptional, translational or post-translational levels.

In further embodiments, the negative modulators can be any type of ligand that specially binds CD5. For example, a dominant negative molecule, mutant CD5, acts as a CD5 decoy receptor, which competes with the inhibitory function of T cell endogenous CD5 in immune responses.

In some instances, genetically modified T cells by inactivating or down-regulating one or more of proteins, such as, CD5, PD1, and CTLA-4 may be used in concert with CARs. A CAR that specifically targets a tumor antigen is then introduced into these modified T cells. The resulting CAR T cells are resistant to T cell exhaustion or inhibition in the tumor microenvironment.

In some special instances, genetically modified T cells by inactivating or down-regulating one or more of genes including, but not limited to, CD5, CTLA-4, and PD1. The resulting modified T cells may be used in concert with CARs specifically targeting the tumor antigen(s). The inactivating or down-regulating CD5 along with CTLA-4 and or PD1 may provide synergistic effects of the prevention of T cell exhaustion or inhibition in the tumor microenvironment.

One aspect of the present disclosure provides for methods, compositions, method of manufacturing, and/or use of CD5 antibody antagonist downregulate or reduce the quantity of the CD5 present on cell surface. CD5 antibody antagonist is linked to the transmembrane via a hinge region

In some embodiments, the present disclosure relates to a method and compositions of enhancing T cell responses by employing an antagonist that reduces inhibitory signal transduction in immune cells. In another embodiment, antagonists can be polypeptides including, but not limited to, CD5 antibody or soluble extracellular portion of CD5. CD5 antibody binding to the T cell CD5 surface molecule, and soluble extracellular CD5 acting as a decoy receptor CD5, prevent the inhibitory signals of T cells being triggered. In a further embodiment, a decoy receptor is referred to the function, which would block the interaction between CD5 and its ligand (s). In further embodiments, the CD5 antibodies may be polyclonal or monoclonal; intact or truncated, e.g. F(ab)2 or scFv; xenogeneic, allogeneic, syngeneic or modified forms thereof.

A representative CDSCAR is encoded by the nucleic acid sequence, SEQ ID NO. 1.

A representative CDSCAR encoded by SEQ ID NO. 1 has amino acid sequence SEQ ID NO. 2.

In some embodiments, CD5 antagonist can be constructed as a membrane-anchored scFv antibody referred to as anchored CD5 scFv antibody. The anchored CD5 scFv is to be expressed on the surface of the T cell and the coding sequence for the scFv is fused in frame with a transmembrane domain via a hinge region and a leader sequence.

In one embodiment, the anchored CD5 scfv antibody is effective to bind to the CD5 protein.

In some embodiments, the transmembrane domain in the anchored CD5 scFv antibody can be selected from, but not limited to, CD28, CD4, CD5, CD7, CD3 epsilon, CD8, CD9, CD16, CD22, CD33, CD137, CD154, CD86, CD41, CD64, and CD68. The transmembrane domain can be a polypeptide, derivative or analogue comprising predominantly hydrophobic residues.

In some embodiments, the anchored CD5 scFv antibody can include one hinge region selected from a group comprising, but is not limited to, CD8a, CD4, immunoglobulin (e.g. IgG1, IgG2, IgG3, IgG4, and IgD).

A representative anchored CD5 scFv antibody is encoded by the nucleic acid sequence SEQ ID NO. 3.

A representative anchored CD5 scFv antibody encoded by SEQ ID NO. 3 has amino acid sequence SEQ ID NO. 4

Enhanced CARs (eCARs)

The provided methods and compositions of the present provides an engineered cell that uses an anchored protein(s) or domains or fragments thereof having an antibody, such as scFv antibody as an enhancer of the anti-tumor immune response, to create more powerful CARs is referred to eCARs. The generated eCARs are more effective at killing cancer cells and stopping tumors, and can retained killing ability within the immunosuppressive tumor environment.

The present disclosure provides a novel enhanced CAR version to combat a key mechanism by which cancer cells resist CAR activity, i.e. the T cell exhaustion or suppression.

In one embodiment, the eCAR includes an anchored CD5 scFv and a CAR via a linker. The linker may be a peptide or a part of a protein, which is self-cleaved after a protein or peptide, is generated (also called a self-cleaving peptide).

In a specific embodiment, eCAR bears the anchored CD5 scFv portion targeting CD5 protein in the T cells, and the CAR portion bearing a chimeric receptor (s) targeting antigen (s) present in the cancer cells. The chimeric receptor(s) in the CAR portion may be for any cancer or any other disease condition for which a specific monoclonal or polyclonal antibody exists or is capable of being generated. In particular, cancers such as neuroblastoma, small cell lung cancer, melanoma, ovarian cancer, renal cell carcinoma, colon cancer, lymphoma, childhood acute lymphoblastic leukemia and blood cancers have antigens specific for the chimeric receptors.

Expression of eCAR may be achieved using, for example, expression vectors including, but not limited to, at least one of a SFFV or human elongation factor 11a (EF) promoter, CAG (chicken beta-actin promoter with CMV enhancer) promoter human elongation factor 1a (EF) promoter, the simian virus 40 (SV40) early promoter, cytomegalovirus (CMV) immediate-early promoter, Ubiquitin C (UBC) promoter, and the phosphoglycerate kinase 1 (PGK) promoter. Expression of eCAR may be achieved using an inducible promoter, including but not limited, a tetracycline responsive promoter, TRE3GV (Tet-response element, including all generations and preferably, the third generation) (Clontech Laboratories, Mountain View, CA).

In some embodiments, eCAR expression in a T cell, includes a high efficiency cleavage site or “self-cleaving” peptide, between the anchored CD5 scFv and CAR. The peptide may be, without limiting, porcine teschovirus-1 2A (P2A), FMDV 2A (abbreviated herein as F2A); equine rhinitis A virus (ERAV) 2A (E2A); and Thoseaasigna virus 2A (T2A) or a combination thereof. Preferably, the “self-cleaving” peptide is P2A.

In some embodiments, an anchored CD5 scFv can be designed to simultaneously express with any one or more of CARs via a self-cleaving peptide as shown in FIG. 13. The targeted cells for CARs may be cancer cells, such as, without limiting, B-cell lymphomas or leukemias, soft tissue tumors. In further embodiments, the target antigens can include at least one of this group, but not limited to, mesothelin, PSCA, WT1, ROR1, PSMA, MAGE A3, Glycolipid, glypican 3, F77, GD-2, CEA, HER-2/neu, MAGE-3, MAGE-4, MAGE-5, MAGE-6, alpha-fetoprotein, CA 19-9, CA 72-4, NY-ESO, FAP, ErbB, c-Met, MART-1, CD30, CD33, CD123, CD19, CD20, CD22, EGFRvIII, immunglobin kappa and lambda, CD38, CD52, CD3, CD4, CD8, CD5, CD7, CD2, and CD138. The target antigens may include viral or fungal antigens, such as E6 and E7 from the human papillomavirus (HPV) or EBV (Epstein Barr virus) antigens.

In some embodiments, eCAR may be expressed in a T cell using bicistronic or multicistronic expression vectors. Several strategies may be employed to construct bicistronic or multicistronic vectors including, but not limited to, (1) multiple promoters fused to the open reading frames; (2) insertion of splicing signals between different portions of the eCAR; (3) insertion of proteolytic cleavage sites between different portions of the eCAR (self-cleavage peptide); and (iv) insertion of internal ribosomal entry sites (IRESs). In one embodiment, one or more proteolytic cleavage sites are inserted at different portions of the eCAR (self-cleavage peptide). Proteolytic cleavage sites have small size and high cleavage efficiency between different portions of eCAR upstream and downstream of the peptide, such as 2A peptide.

In an embodiment, the anchored scFv antibody comprises a CD5 antigen recognition domain, a hinge region, and a transmembrane domain, but lacks co-stimulatory domain(s) and the intracellular domain of CD3 zeta chain.

The CAR portion comprises different or same antigen recognition domain, different or same hinge region, same or different transmembrane domain. In addition, the CAR portion also bears the co-stimulatory domain (s) and intracellular domain of CD3 zeta chain, which are not included in the anchored CD5 scFv antibody portion.

The disclosure includes a method of generating an eCAR. In some embodiments, the eCAR is generated using T-cells. The T cells may be isolated from any type of cells, such as the peripheral blood, cord blood, bone marrow and tumor infiltrating lymphocytes.

A method for treating cancers using eCAR in a subject is embodied in the present disclosure. The method comprises:
(1) Obtaining T cells from a subject or donor(s).
(2) Culturing the lymphocytes/T cells
(3) Introducing an eCAR construct into the cultured T cells.
(4) Expanding eCAR T cells and monitoring CD5 negative eCAR T population
(5) Treating the subject with lymphodepletion chemotherapeutic agents
(6) Administrating the CD5 negative eCAR T cells or other mixture of CD5 positive or negative cells to the subject.

The ex vivo expansion of tumor-infiltrating lymphocytes (TILs) are successfully used in the current adoptive cell therapy. In one embodiment, TILs are harvested and successfully expanded ex vivo.

CD5 is a negative modulator of T cell activation, and thus plays a key role in preventing activation-induced cell death. CD5-deficient mice have shown the delayed tumor growth. TILs lacking CD5 expression exhibit a more highly activated phenotype and enhanced ex vivo antitumor cytotoxicity and cytokine responses than TILs expressing CD5.

In some embodiments, TILs can be obtained from a tumor tissue sample and expanding the umber of TILs. The anchored CD5 scFv construct can be introduced into TILs using any one of means described above and the anchored CD5 scFv is expressed on the surface of TILs. The anchored CD5 scFv downregulates or reduces the quantity of the CD5 presented on cell surface of TILs, which may enhance their responses to cancers, which is valuable to the disease therapies.

### CD123 CAR NK Cells

Another potential roadblock to successful CAR therapy are the known safety issues associated with this type of immune therapy, of which the most common are cytokine storm, tumor lysis syndrome, and on-target, off tumor effects. One way to address this potential issue is to utilize immune cell types other than the T cell to develop CARs for therapy. Natural-Killer (NK) cells are a critical subset of the innate immune system that recognize and kill both virally infected and malignant cells without the requirement for prior sensitization. Because NK cells will not clonally expand upon activation, they can potentially avoid the issue of cytokine storm, tumor lysis syndrome and off tumor effects in a clinical setting, though this may require a trade-off in efficacy. CAR NK cells may also be used in the setting of allogenic transplantation without risk of graft versus host disease (GVHD), while maintaining functional graft versus tumor effects. NK cell CARs have also been developed for multiple diseases, including hematological malignancies with a reassuring safety profile in phase 1 trials. For these reasons, NK cell CARs are a promising route to the development of safe and effective CAR therapies for hematological malignancies.

CD123 is the alpha chain of the interleukin 3 receptor and is overexpressed on a variety of hematologic malignancies, including acute myeloid leukemia (AML), B-cell acute lymphoblastic leukemia (B-ALL), hairy cell leukemia, and blastic plasmocytoid dendritic neoplasms (Testa, Pelosi et al. 2014). More importantly, CD123 is expressed on a subset of leukemic cells related to leukemic stem cells (LSCs), the ablation of which is essential in preventing disease refractoriness and relapse.

The clinical outcome for AML patients correlates with number of CD34+/CD38-/CD123+ LSCs. The CD34+/CD38−/CD123+ cells, greater than 15% in AML patients, are associated with a lack of complete remission and unfavorable cytogenetic profiles. In addition, the presence of more than 1% of CD34+/CD38−/CD123+ cells could also have a negative impact on disease-free survival and overall survival.

The present disclosure relates to the use of NK cells engineered to express a CAR to treat a disease associated with CD123 expression. The disease treated by CD123CAR NK cells, may include one or more of, but not limited to, ALL, acute myeloid leukemia (AML), chronic myeloid leukemia, chronic myeloproliferative neoplasms, blastic plasmacytoid dendritic cell neoplasm, hairy cell leukemia and myelodysplastic syndromes (MDS).

CD123CAR includes an anti-CD123 binding domain and at least one of intracellular signaling, hinge and/or transmembrane domains. First-generation CD23 CAR may include CD3z as an intracellular signaling domain, whereas second-generation CD123CARs include at least one single co-stimulatory domain derived from, for example, without limiting, CD28 and/or 4-1BB. Third generation CD123 CAR may include two co-stimulatory domains, such as, without limiting, CD28, 4-1BB, and any other co-stimulatory molecules.

A representative CD123CAR is encoded by the nucleic acid sequence SEQ ID NO. 5.

A representative CD123CAR antibody has amino acid sequence SEQ ID NO. 6

### CD3 CAR NK Cells

CD3 is the common marker for T cells and T cell malignancies. OK3 against CD3 epsilon is the common antibody used for identifying T cells. Anti-CD3 monoclonal antibodies as treatments include: (1) acute renal, cardiac or hepatic allograft rejection; (2) Depletion of T cells from donor marrow prior to transplant; (3) new onset of type I diabetes. CD3 against CD3 epsilon chain is the most specific T cell antibody used to identify T cells in benign and malignant disorders. Studies have shown that CD3 was found in 86% of peripheral T cell lymphomas

In some embodiments, the NK cell bearing the CD3 CAR exhibits an antitumor immunity and exerts the efficacy of killing leukemias/lymphomas expressing CD3.

The disclosure relates to the methods for deletion or reducing abnormal or malignant T cells in bone marrow, blood and organs using CD3CAR NK cells. In some embodiments, CD3 positive malignancies may include, but is not limited to precursor T lymphoblastic leukemia/lymphoma, mature T cell lymphomas/leukemias, EBV-positive T-cell lymphoproliferative disorders, adult T-cell leukemia/lymphoma, mycosis fungoides/sezary syndrome, primary cutaneous CD30-positive T-cell lymphoproliferative disorders, peripheral T-cell lymphoma (not otherwise specified), angioimmunoblastic T-cell lymphoma and anaplastic large cell lymphoma.

In some embodiments, CD3CAR NK cells can be used to treat patients with T-leukemias/lymphomas, which are not eligible for stem cell therapy or never achieved a remission despite many intensive chemotherapy regimens. In further embodiments, CD3CARNK cells may be used as a component of conditioning regimen for a bone marrow transplant or a bridge to the bone marrow transplant.

A representative CD3CAR is encoded by the nucleic acid sequence SEQ ID NO. 7.

A representative CD3CAR encoded by SEQ ID NO. 7 has amino acid sequence SEQ ID NO. 8.

As used herein, “patient” includes mammals. The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human. A patient includes subject.

In certain embodiments, the patient is a human 0 to 6 months old, 6 to 12 months old, 1 to 5 years old, 5 to 10 years old, 5 to 12 years old, 10 to 15 years old, 15 to 20 years old, 13 to 19 years old, 20 to 25 years old, 25 to 30 years old, 20 to 65 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old.

The terms “effective amount” and “therapeutically effective amount” of an engineered cell as used herein mean a sufficient amount of the engineered cell to provide the desired therapeutic or physiological or effect or outcome. Such, an effect or outcome includes reduction or amelioration of the symptoms of cellular disease. Undesirable effects, e.g. side effects, are sometimes manifested along with the desired therapeutic effect; hence, a practitioner balances the potential benefits against the potential risks in determining what an appropriate “effective amount” is. The exact amount required will vary from patient to patient, depending on the species, age and general condition of the patient, mode of administration and the like. Thus, it may not be possible to specify an exact “effective amount”. However, an appropriate “effective amount” in any individual case may be determined by one of ordinary skill in the art using only routine experimentation. Generally, the engineered cell or engineered cells is/are given in an amount and under conditions sufficient to reduce proliferation of target cells.

Following administration of the delivery system for treating, inhibiting, or preventing a cancer, the efficacy of the therapeutic engineered cell can be assessed in various ways well known to the skilled practitioner. For instance, one of ordinary skill in the art will understand that a therapeutic engineered cell delivered in conjunction with the chemo-adjuvant is efficacious in treating or inhibiting a cancer in a patient by observing that the therapeutic engineered cell reduces the cancer cell load or prevents a further increase in cancer cell load. Cancer cell loads can be measured by methods that are known in the art, for example, using polymerase chain reaction assays to detect the presence of certain cancer cell nucleic acids or identification of certain cancer cell markers in the blood using, for example, an antibody assay to detect the presence of the markers in a sample (e.g., but not limited to, blood) from a subject or patient, or by measuring the level of circulating cancer cell antibody levels in the patient.

Throughout this specification quantities are defined by ranges, and by lower and upper boundaries of ranges. Each lower boundary can be combined with each upper boundary to define a range. The lower and upper boundaries should each be taken as a separate element.

Reference throughout this specification to “one embodiment,” “an embodiment,” “one example,” or “an example” means that a particular feature, structure or characteristic described in connection with the embodiment or example is included in at least one embodiment of the present embodiments. Thus, appearances of the phrases “in one embodiment,” “in an embodiment,” “one example,” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus.

Further, unless expressly stated to the contrary, “or” refers to an inclusive “or” and not to an exclusive “or”. For example, a condition A or B is satisfied by any one of the following: A is true (or present) and B is false (or not present), A is false (or not present and B is true (or present), and both A and B are true (or present).

Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e” and “in one embodiment”

In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any one, two, three, or four of the members, e.g., a and c; a, d, and e; b, c, d, and e; etc.

As used herein, a XXXX antigen recognition domain is a polypeptide that is selective for XXXX. “XXXX” denotes the target as discussed herein and above. For example, a CD38 antigen recognition domain is a polypeptide that is specific for CD38.

As used herein, CDXCAR refers to a chimeric antigen receptor having a CDX antigen recognition domain.

The present disclosure may be better understood with reference to the examples, set forth below. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure.

### Examples

### Generation of the Third Generation of CDSCAR

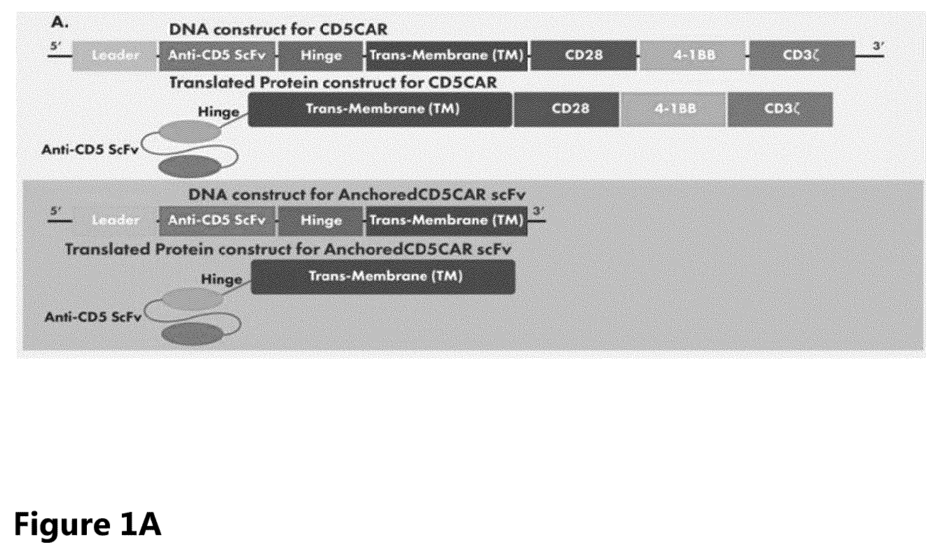

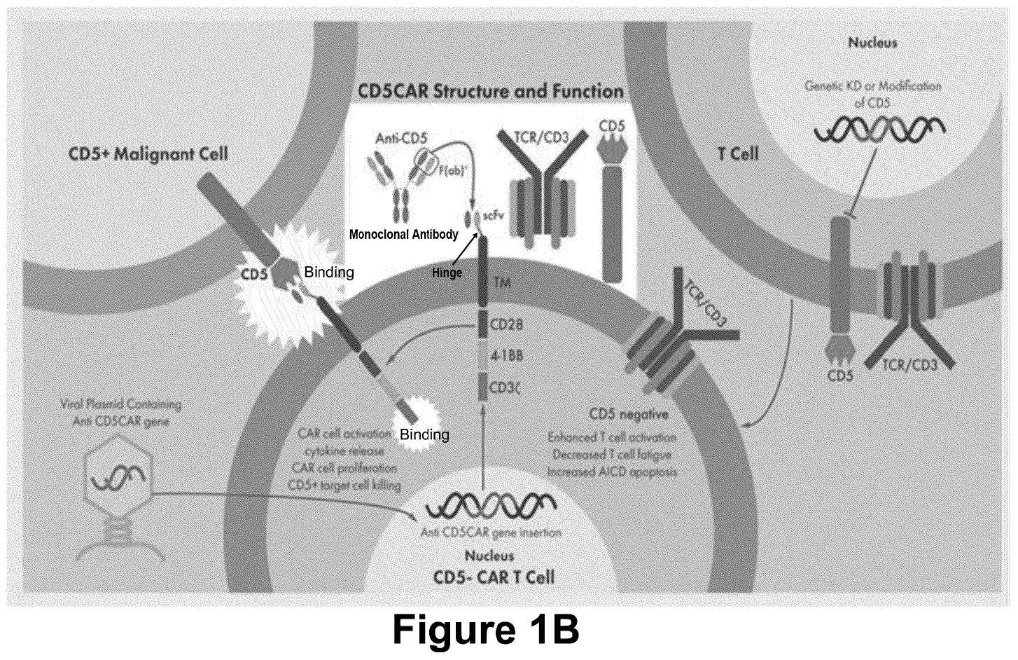

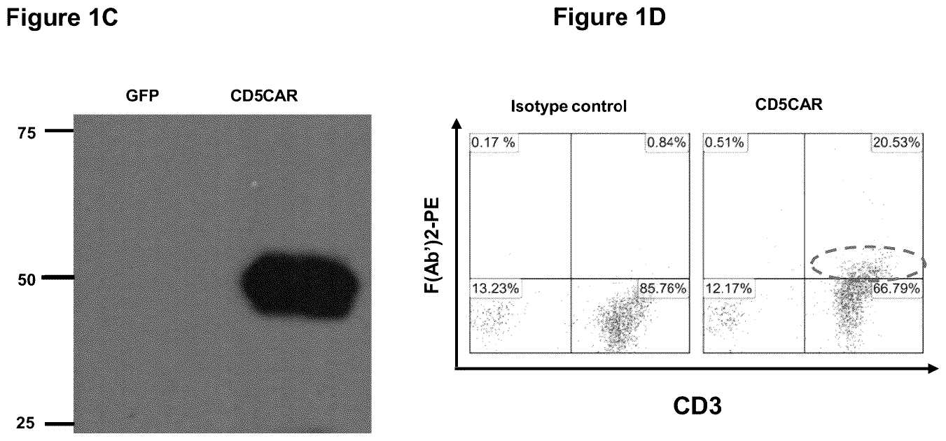

The construct for CD5 CAR, as well as anchored CD5 scFv antibody were designed to test the function and mechanism of CDSCAR T cells in terms of both the targeting and lysis of CD5 expressing cells and the ability of CDSCAR T cells to down-regulate CD5 expression within their own CDSCAR T cell population (FIG. 1A). To confirm the CDSCAR construct, the generated CDSCAR lentiviruses were transduced into HEK293 cells. After 48h treatment with CDSCAR or GFP-lentiviruses, the expression of CDSCAR in HEK293 cells was verified by Western blot analysis using CD3□ antibody which recognize C-terminal region of CDSCAR protein (FIG. 1B). The resulting band was the predicted size of CDSCAR protein in CDSCAR transduced HEK293 cells, but GFP transduced HEK293 cells did not exhibit any specific band by Western blot analysis. In order to evaluate the function of CDSCAR protein for future experiments, CDSCAR lentiviruses were transduced into activated human T cells. The expression of CDSCAR on surface of T cells was evaluated by flow cytometry analysis using goat anti-mouse F(ab′) antibody, which recognize scFv region of CDSCAR protein. Flow cytometric analysis showed that about 20% of CDSCAR expression was observed on CDSCAR transduced T-cells compared to isotype control (FIG. 1C). These results indicated that we succeed to generate CDSCAR expression T cell for following experiments.

### The Down-Regulation of CD5 Expression for CAR Therapy

Prior to CDSCAR T cell co-culture with MOLT-4 and CCRF-CEM cell lines, the expression of CD5 on the surface of CDSCAR T cells is down regulated to avoid self-killing within the CDSCAR population. The down-regulation of CD5 will prevent the self-killing of CAR T cells within the CAR T cell population, and the down-regulation of CD5 is associated with an increased killing ability of T-cells. A CAR that is produced within T-cells that has no CD5 expression could be a super-functional CAR, no matter the construct of the CAR itself. Initially, down-regulation of CD5 was accomplished through the stable knockdown (KD) of CD5 for both cell lines using two CDISPR/Cas9 KD sequences that differed in the choice of leader sequence (FIGS. 4A and 4C). The most successful population in terms of CD5 downregulation was chosen for each cell line, and these cells were sorted and found to be of >99% purity CD45+ and CD5-(FIGS. 4B and 4D).

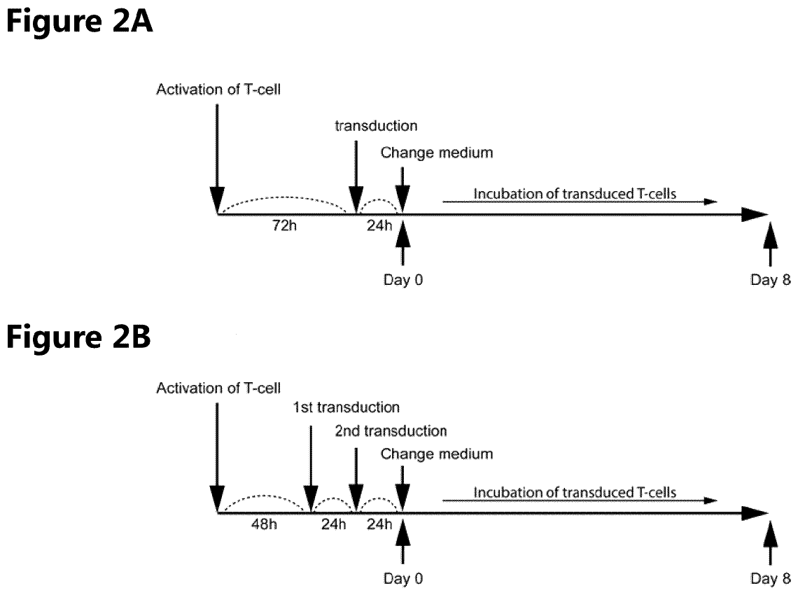

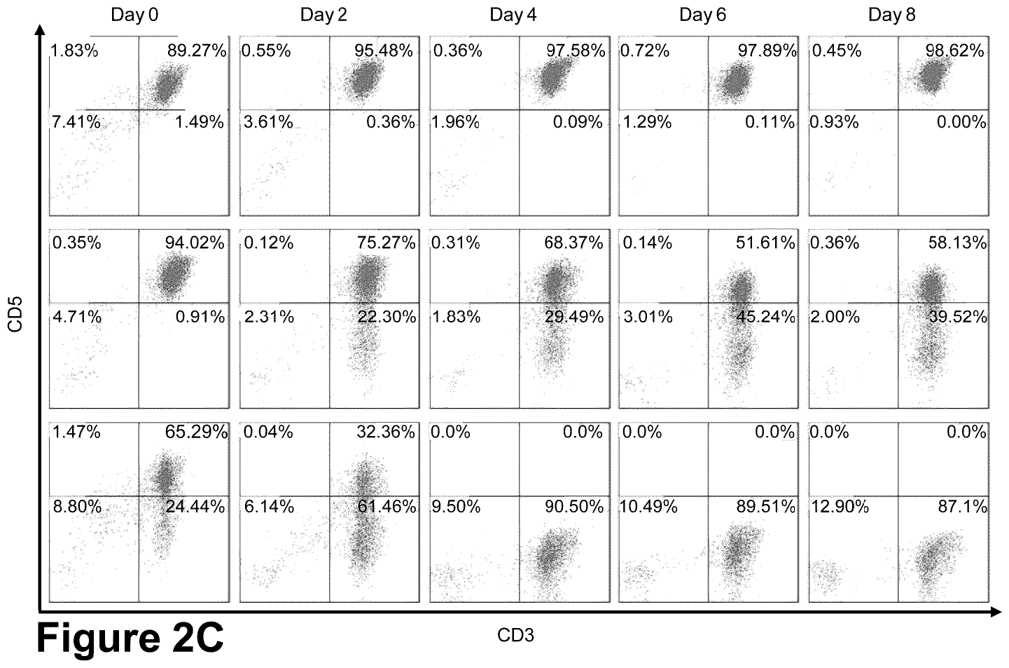

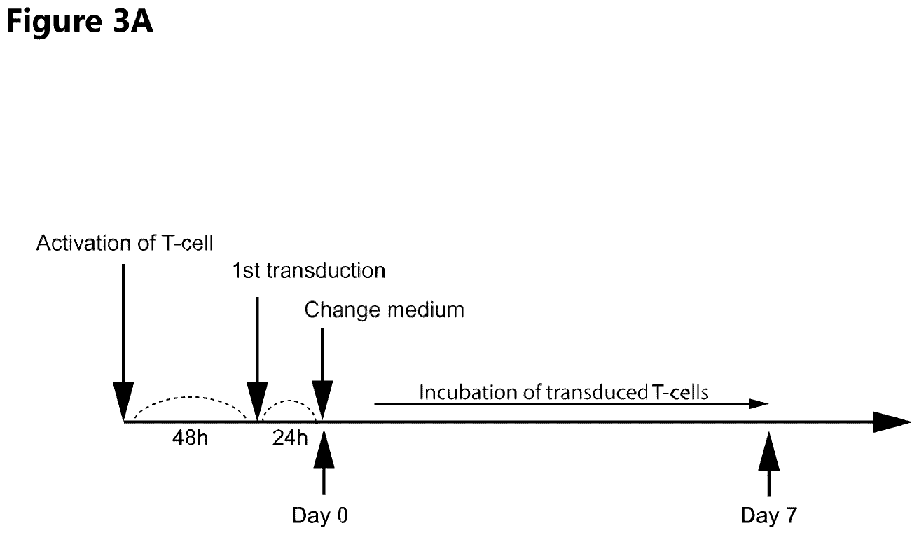

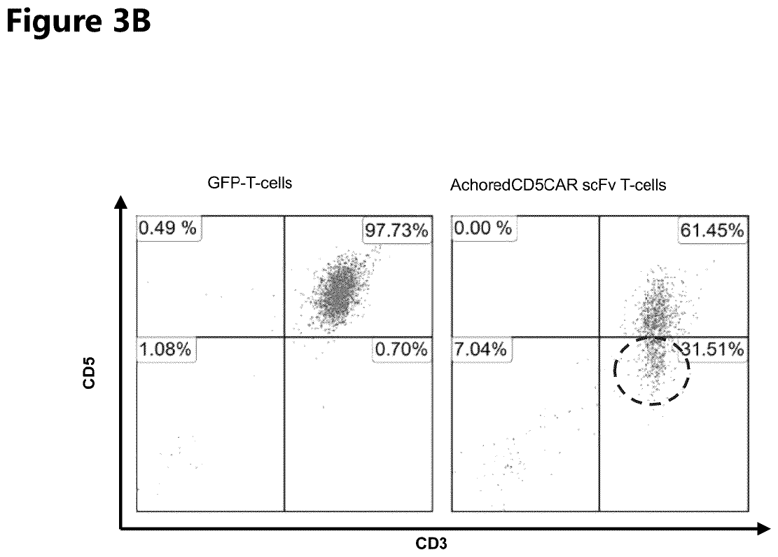

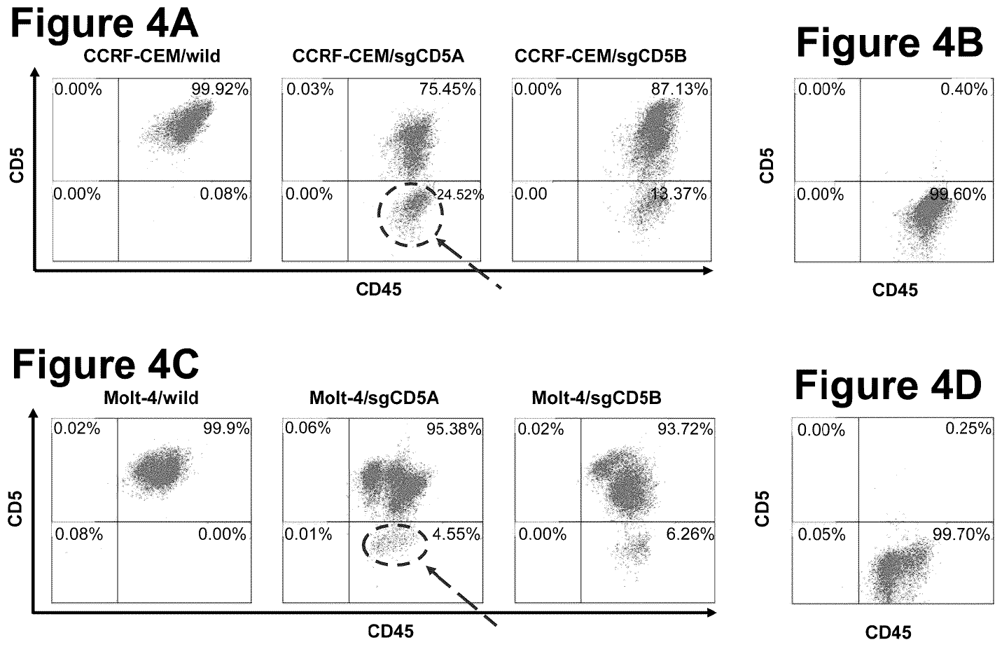

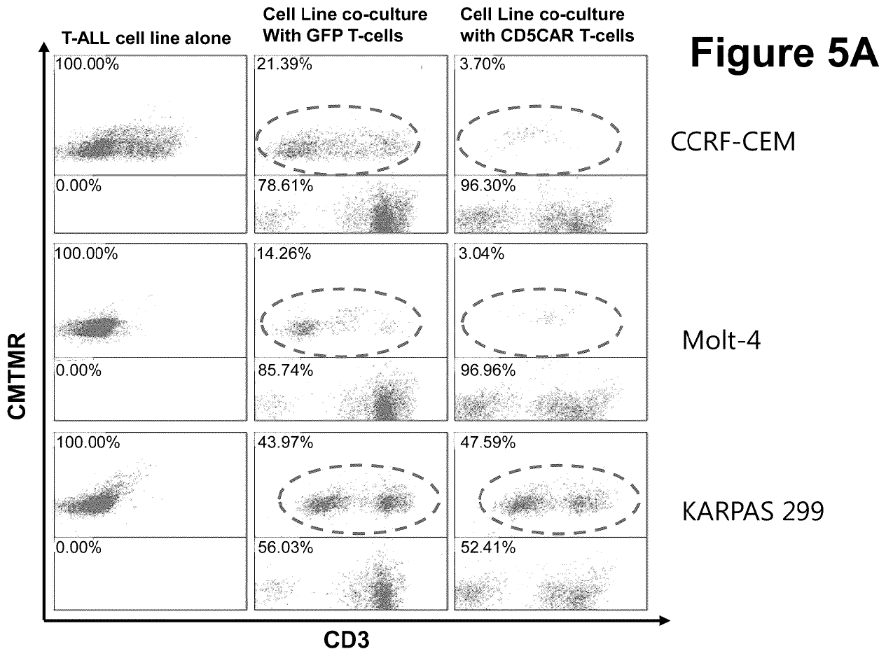

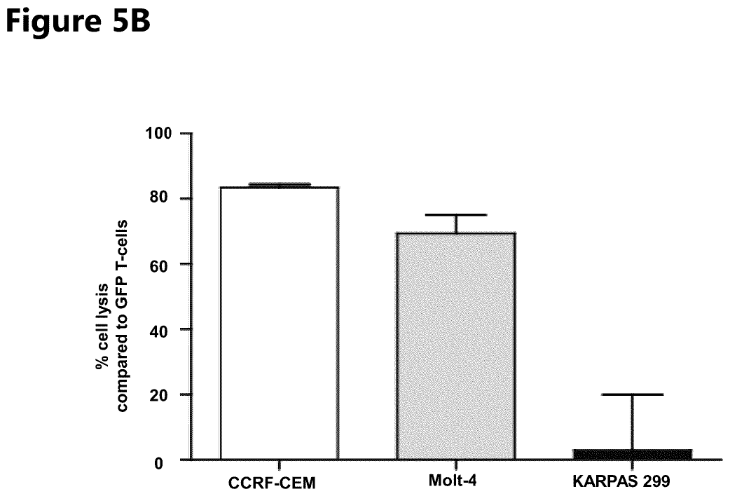

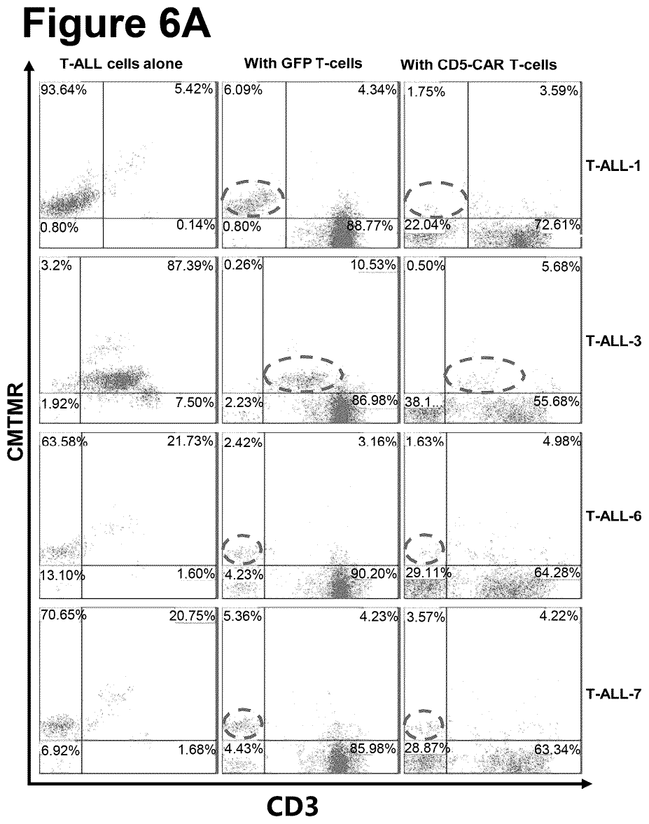

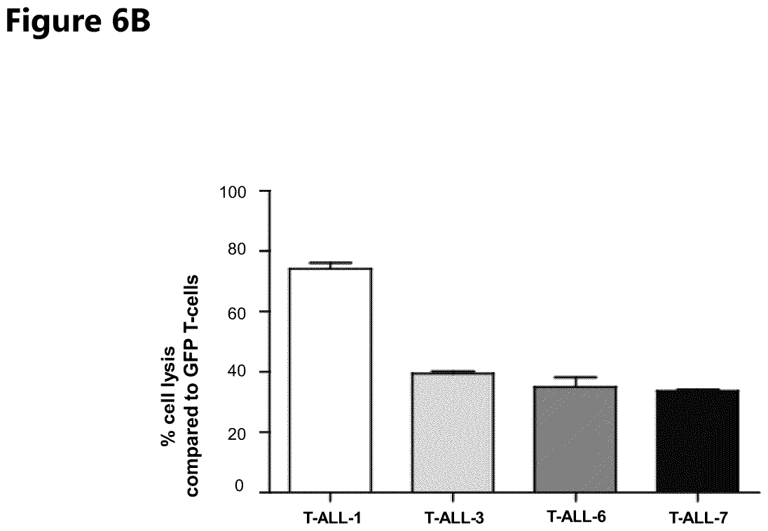

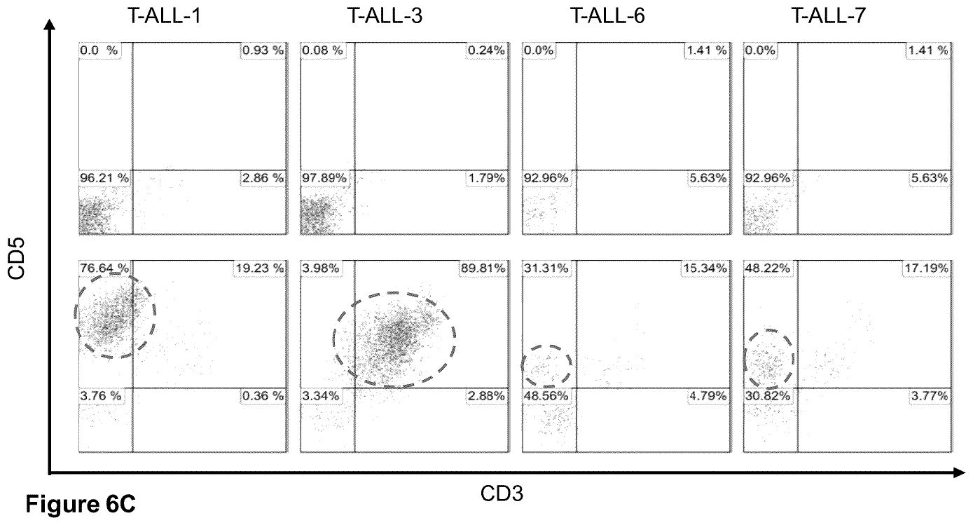

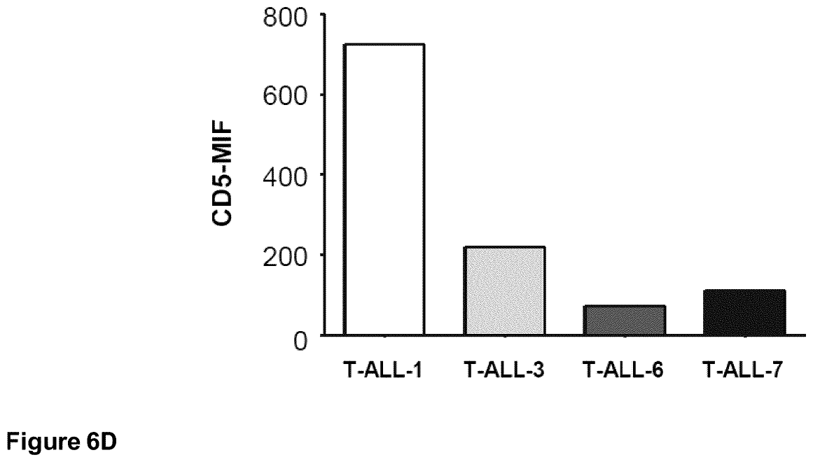

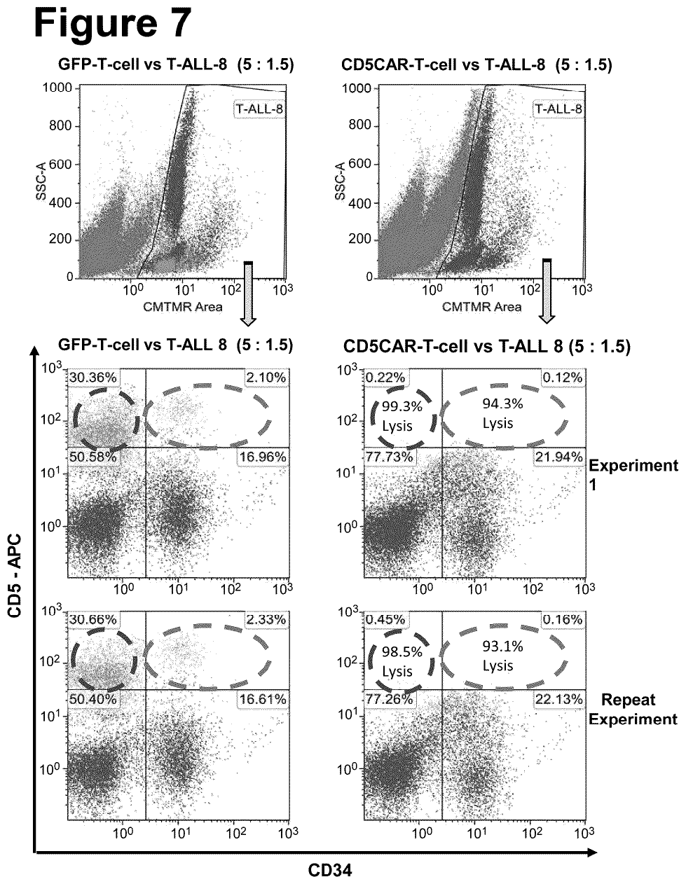

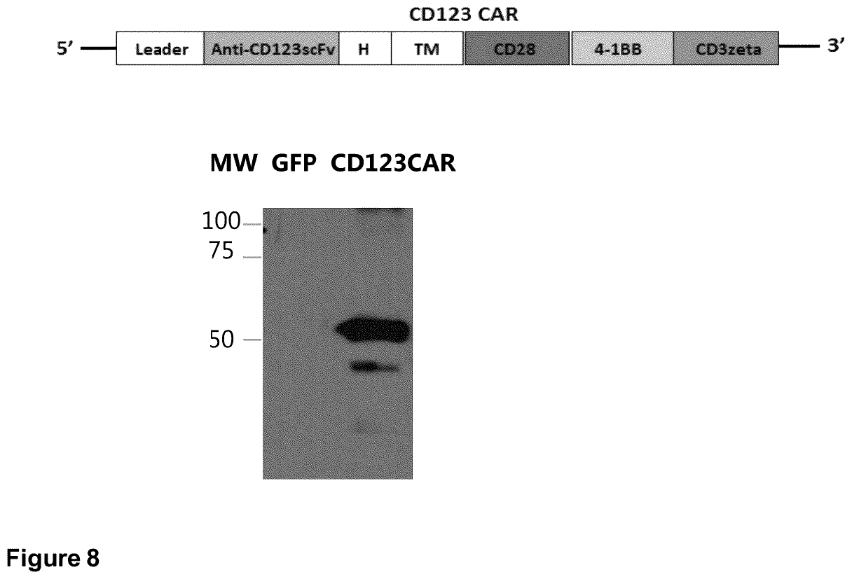

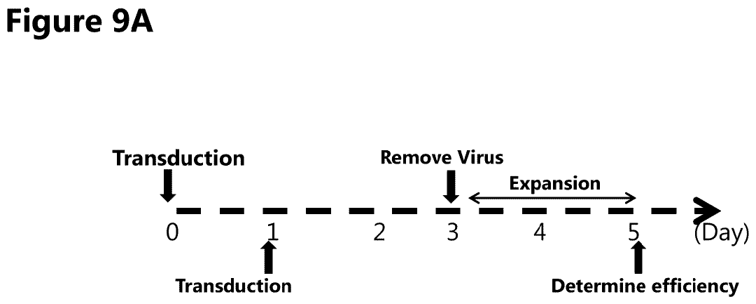

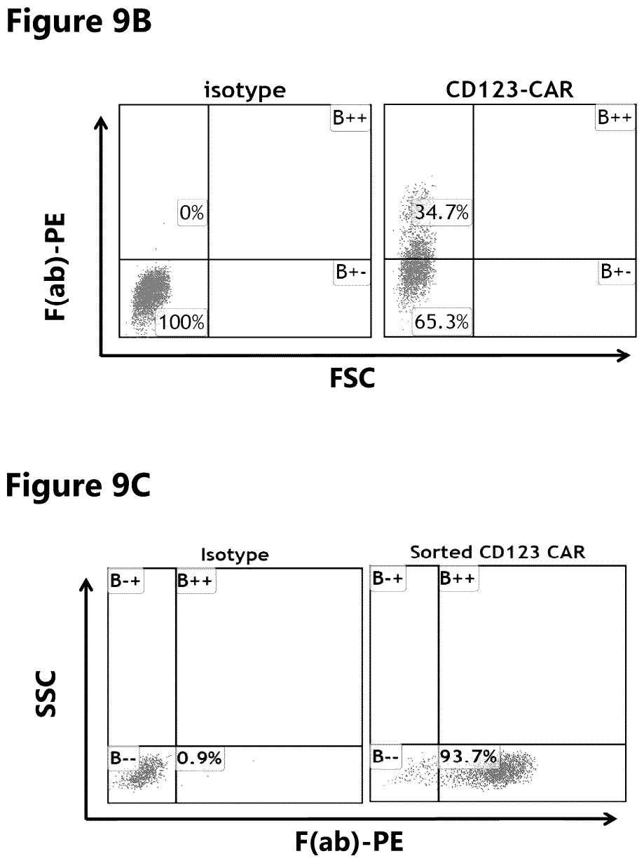

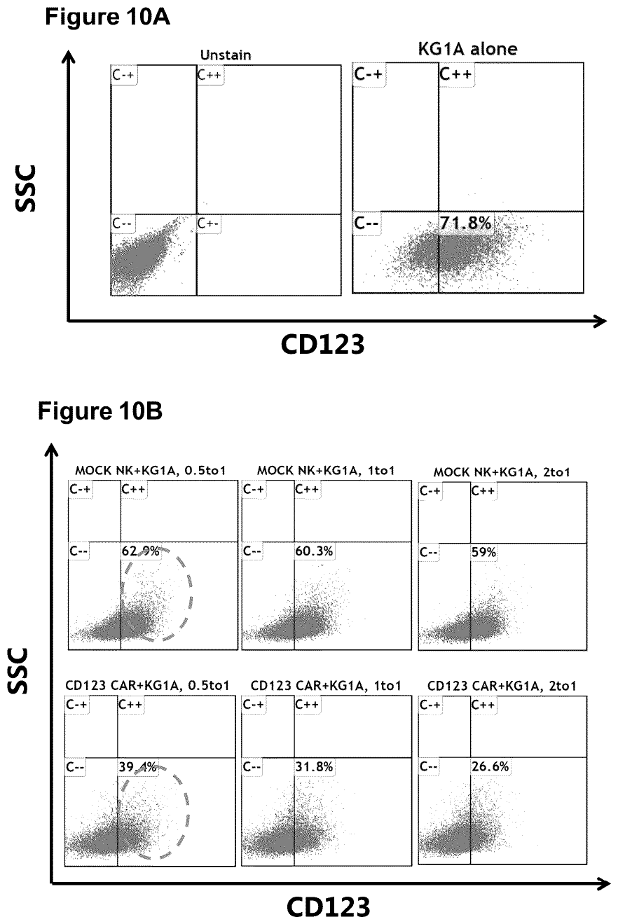

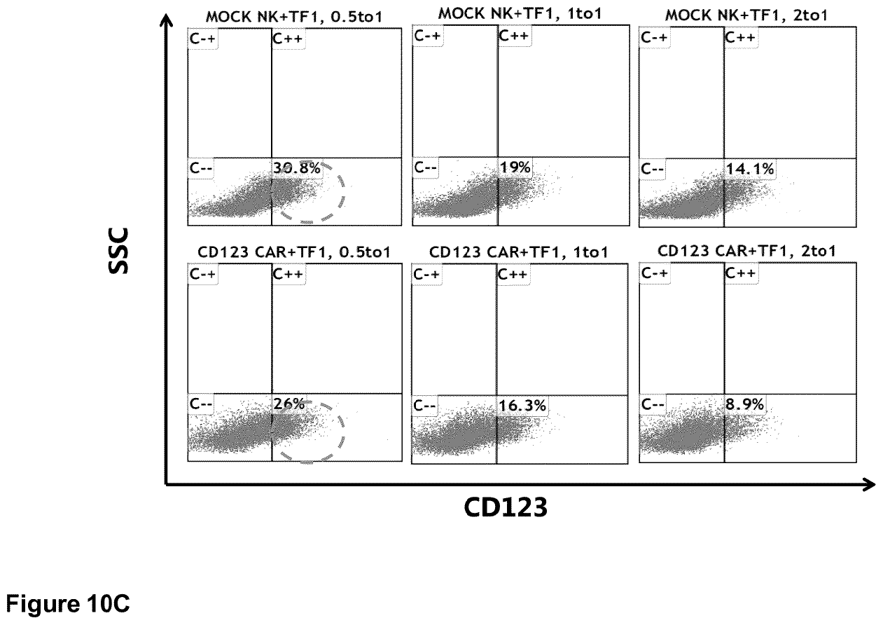

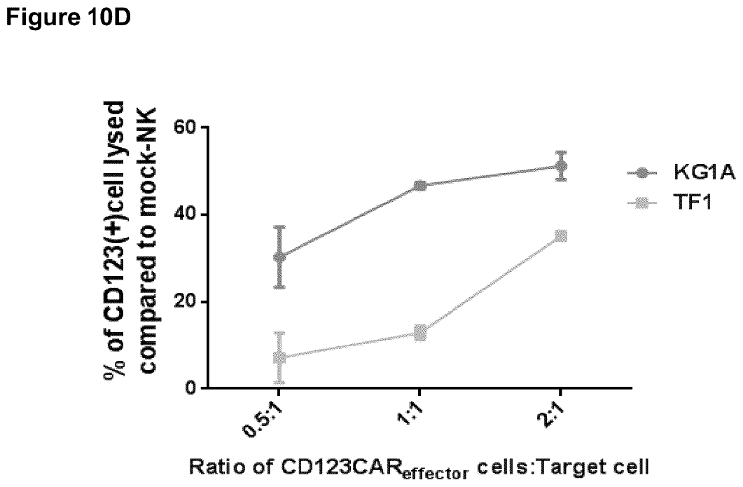

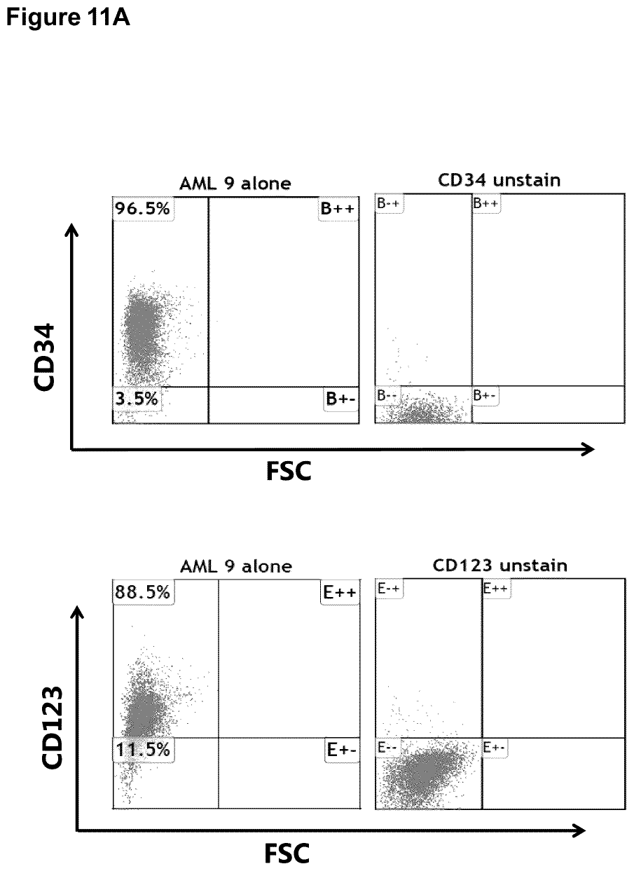

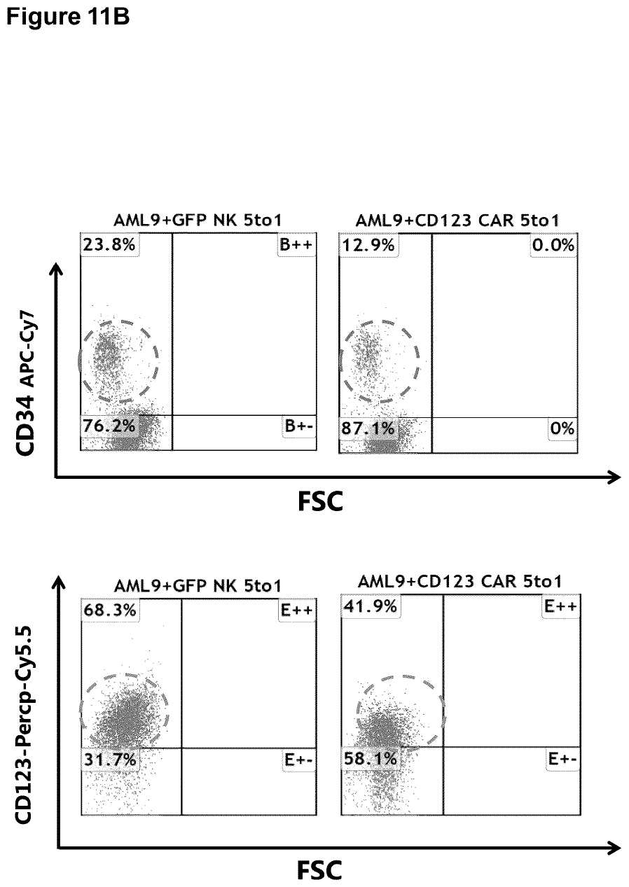

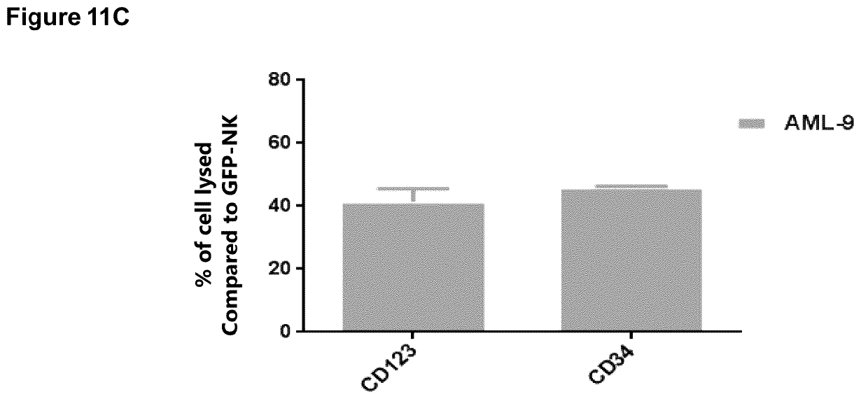

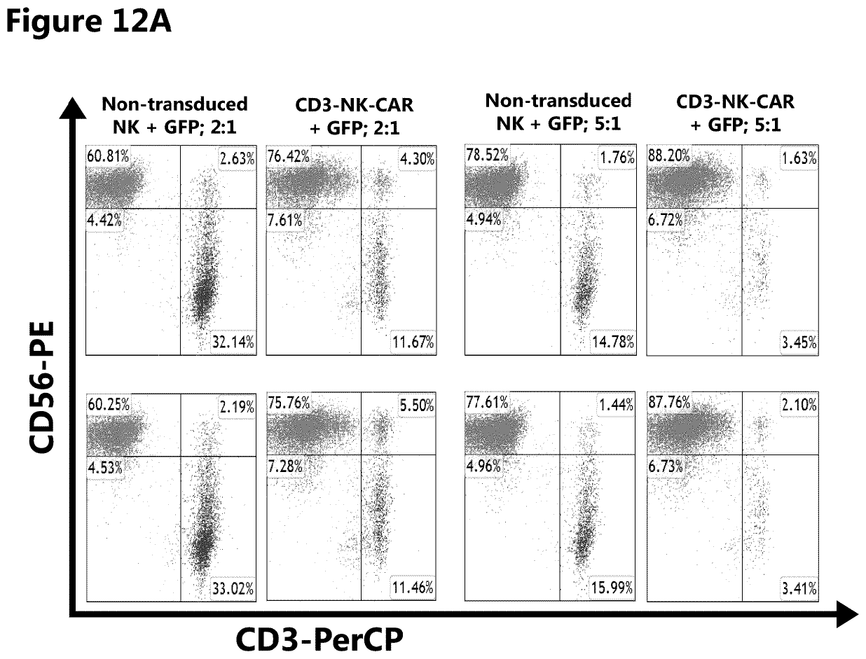

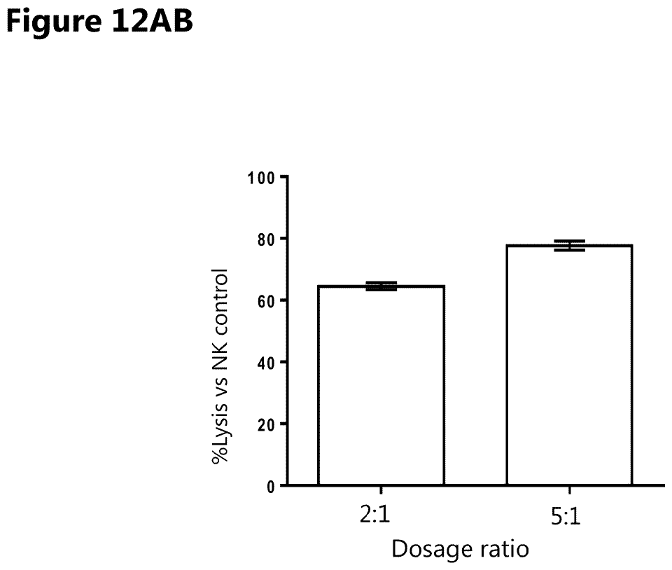

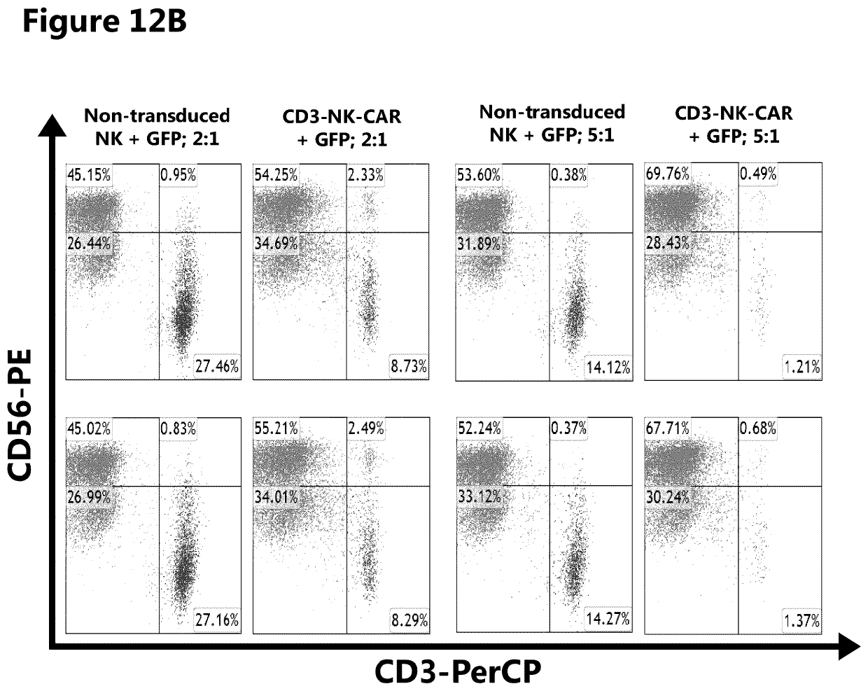

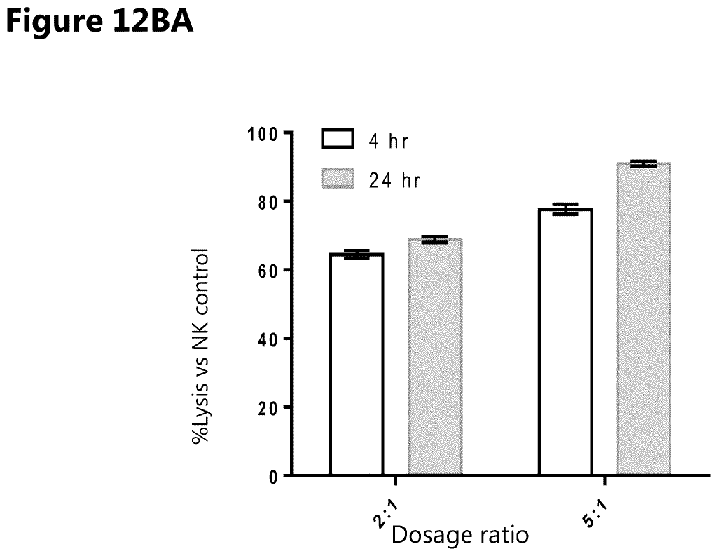

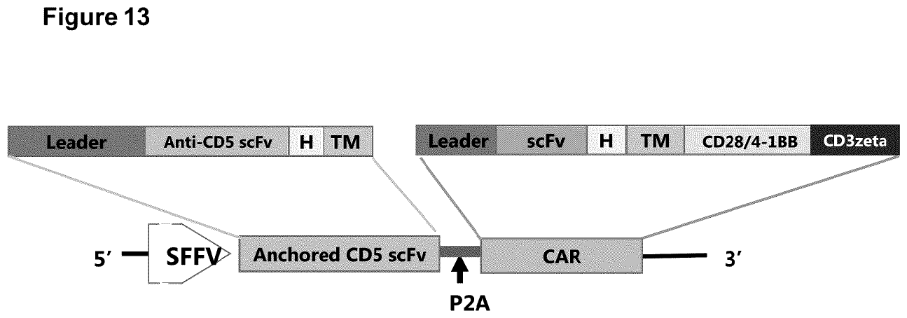

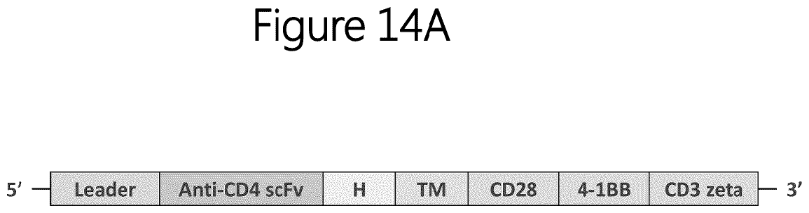

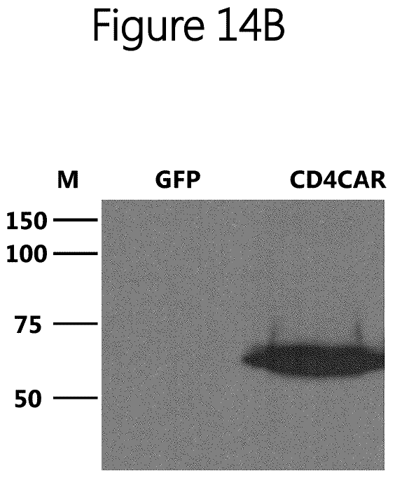

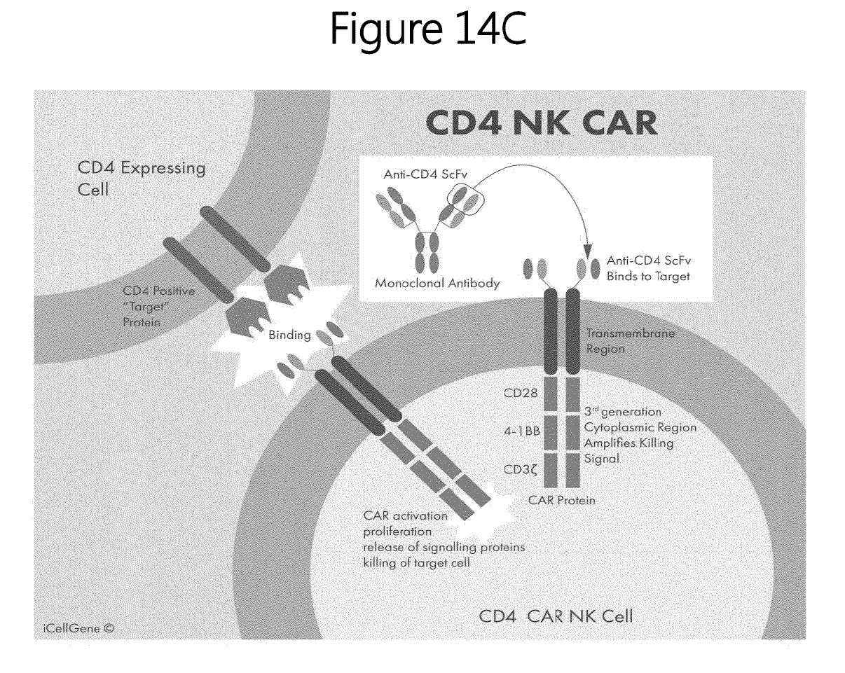

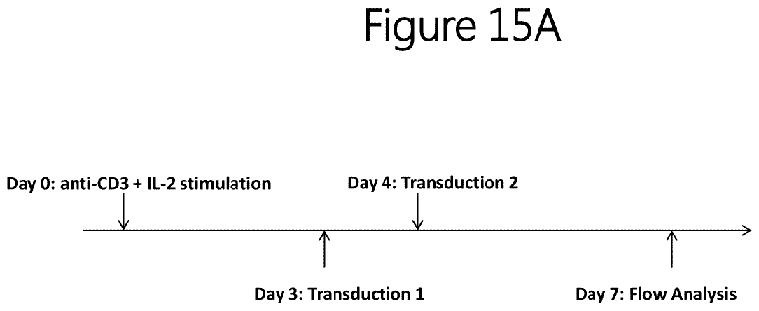

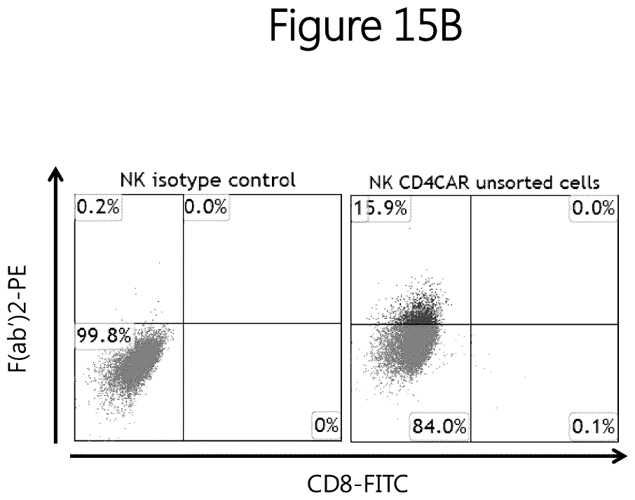

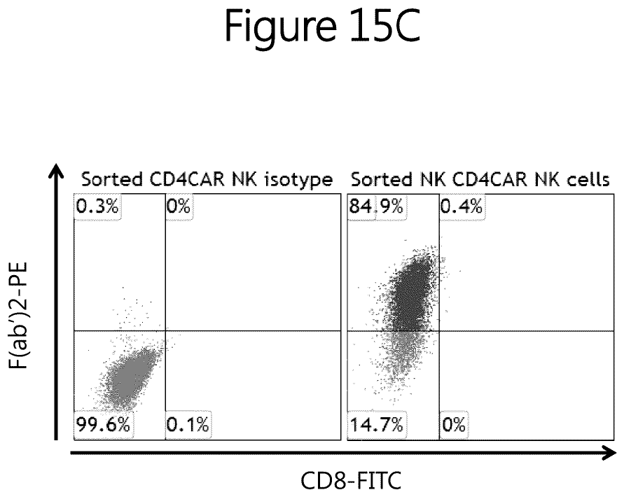

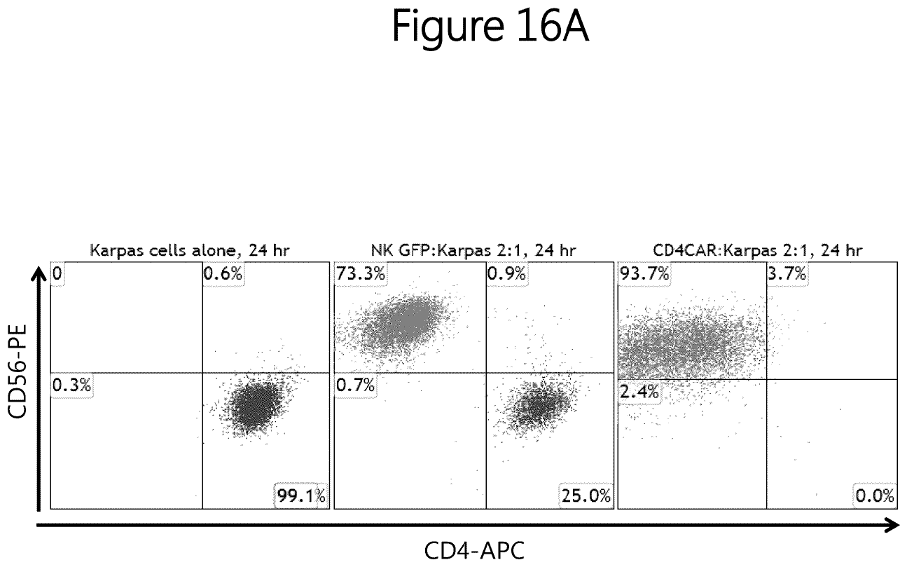

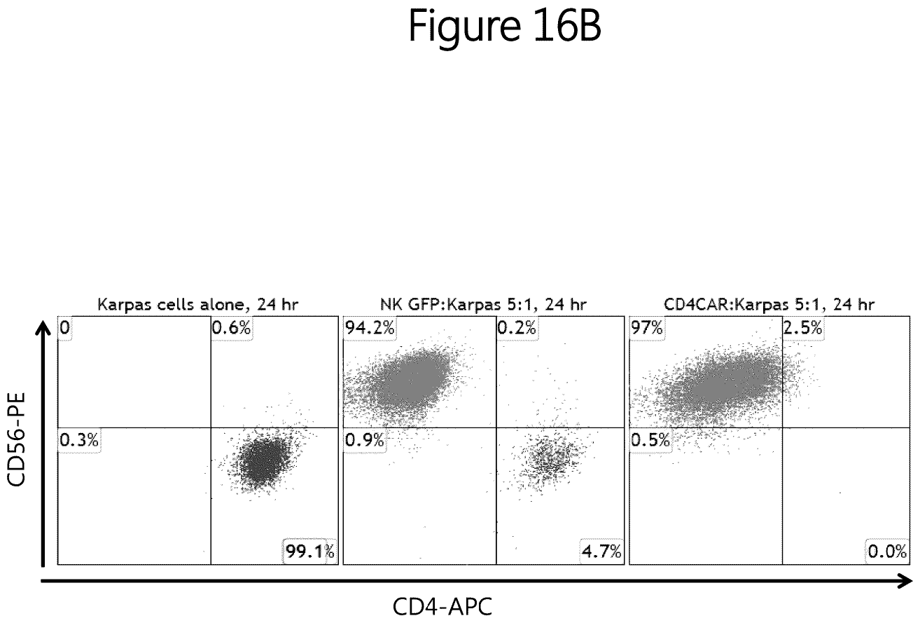

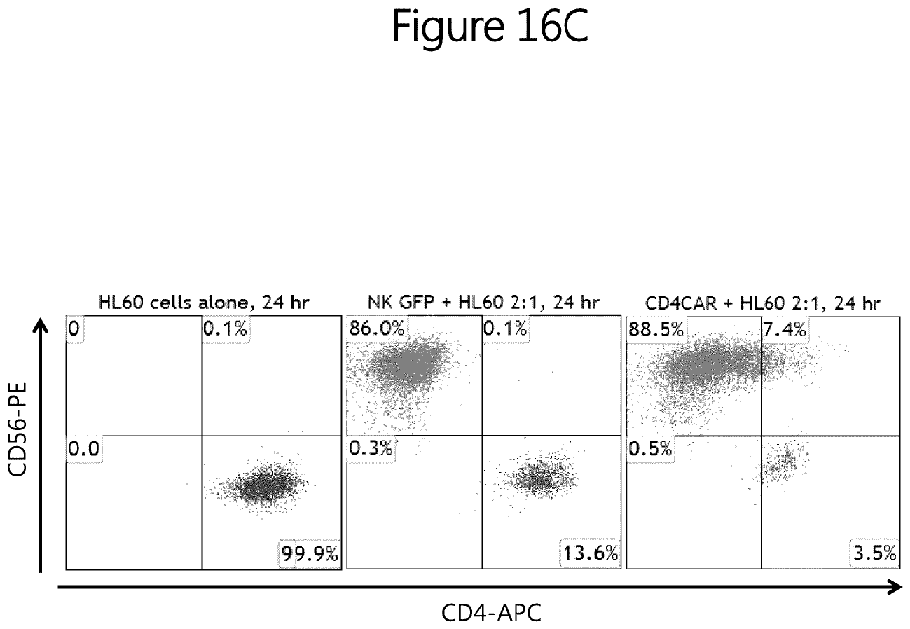

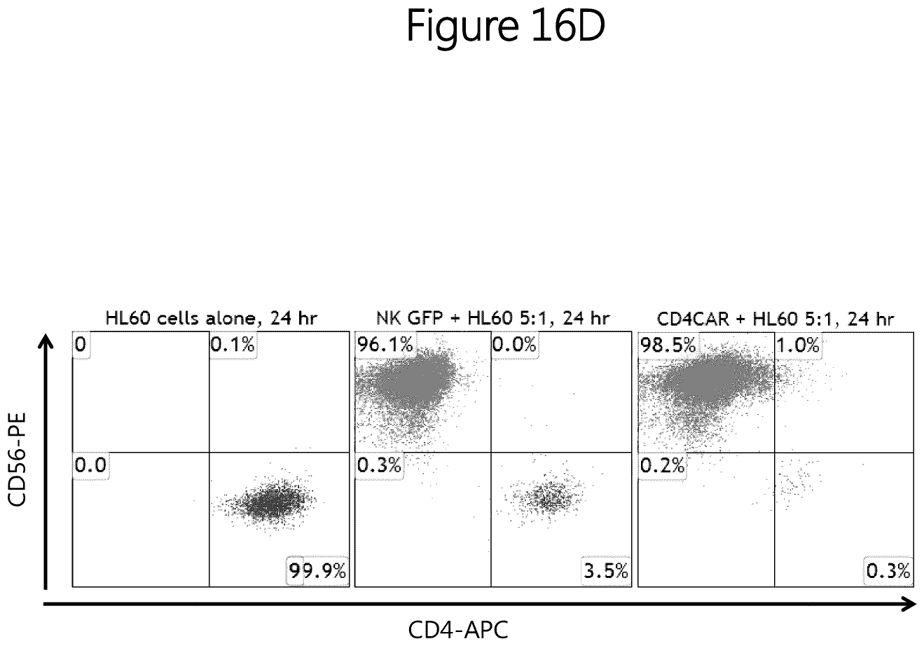

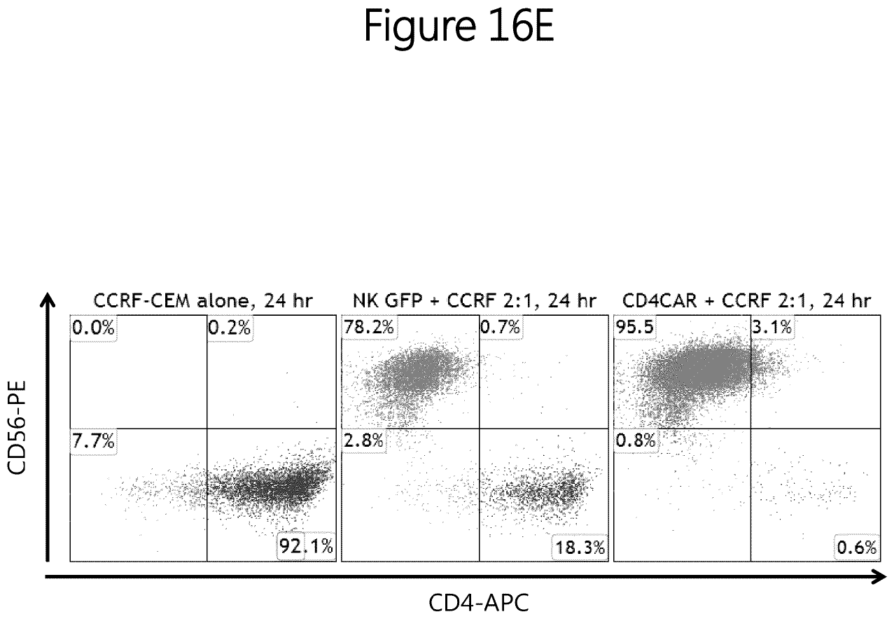

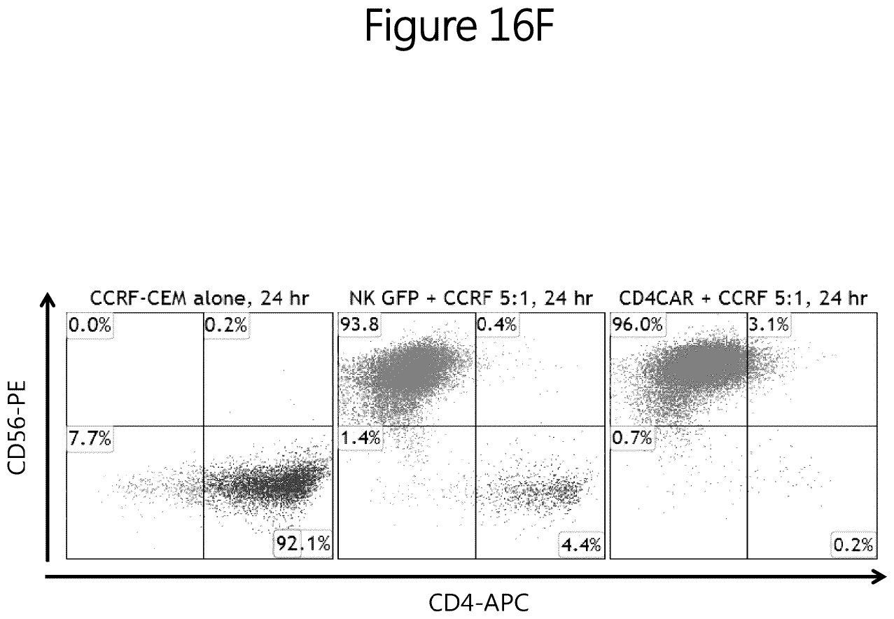

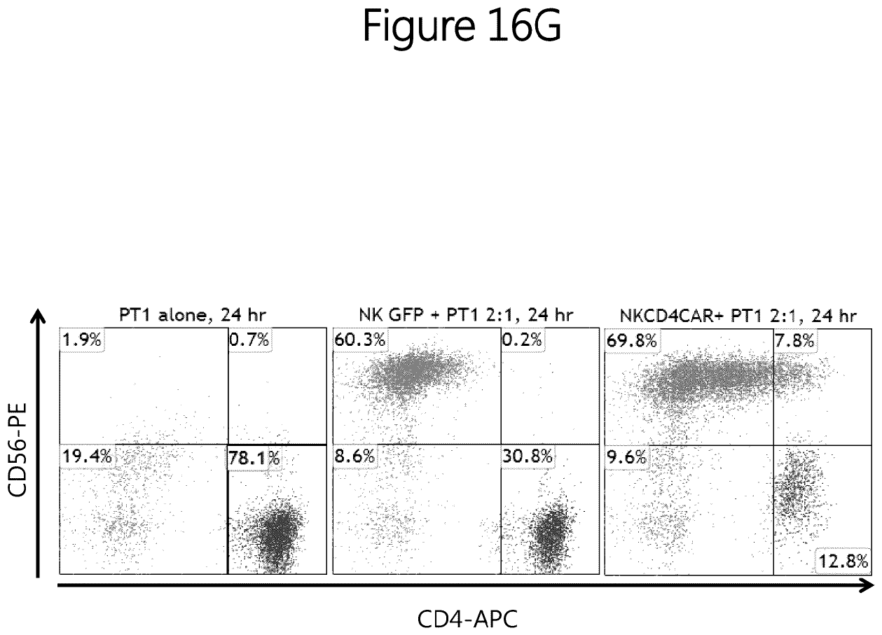

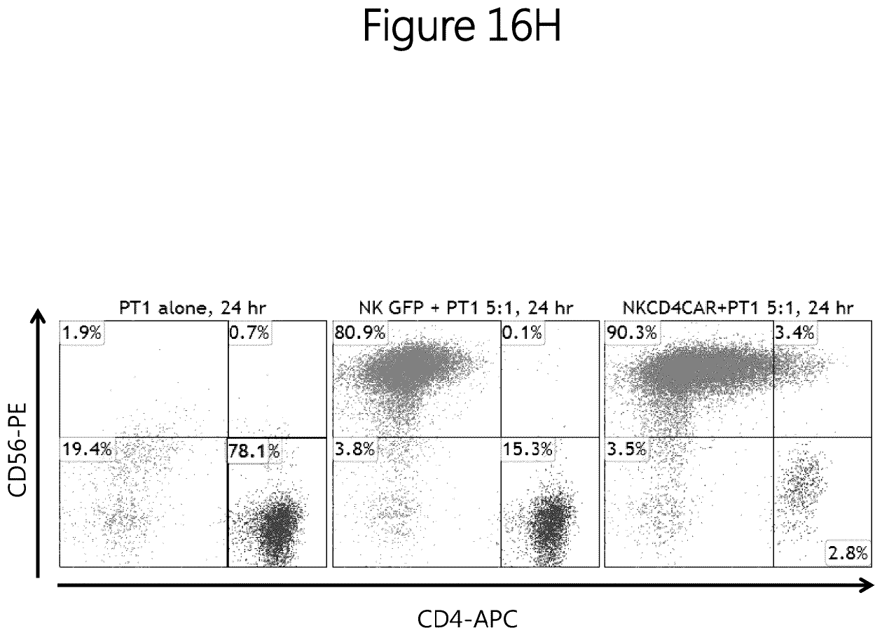

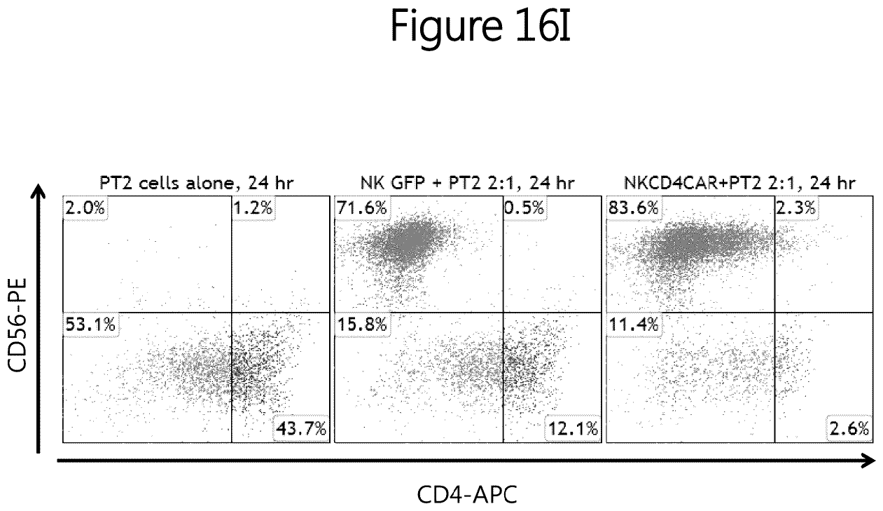

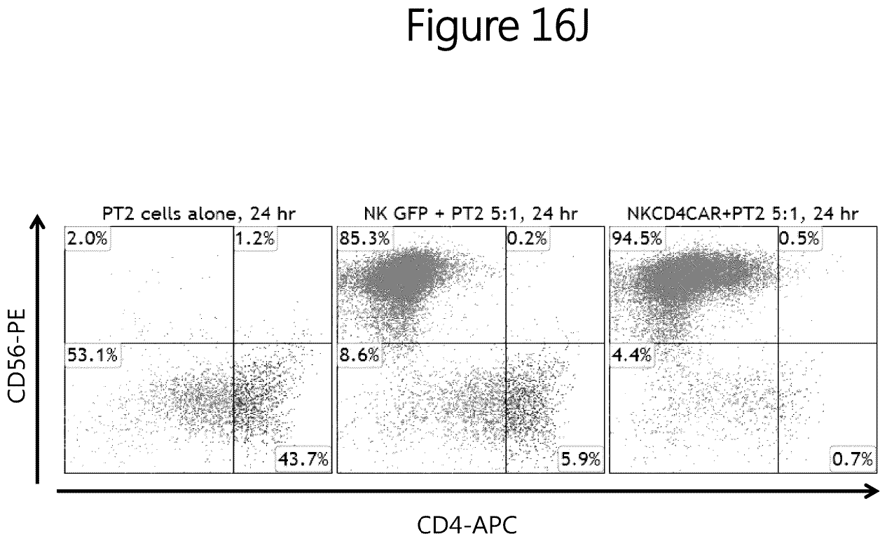

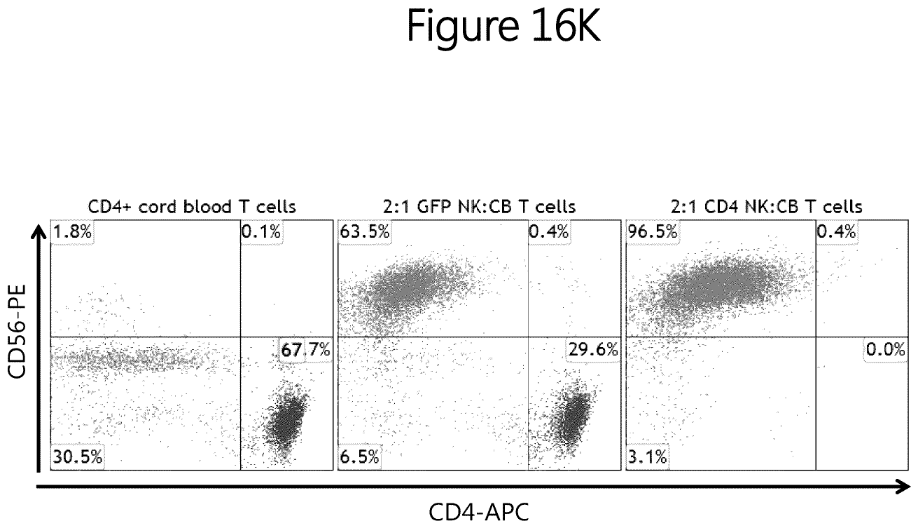

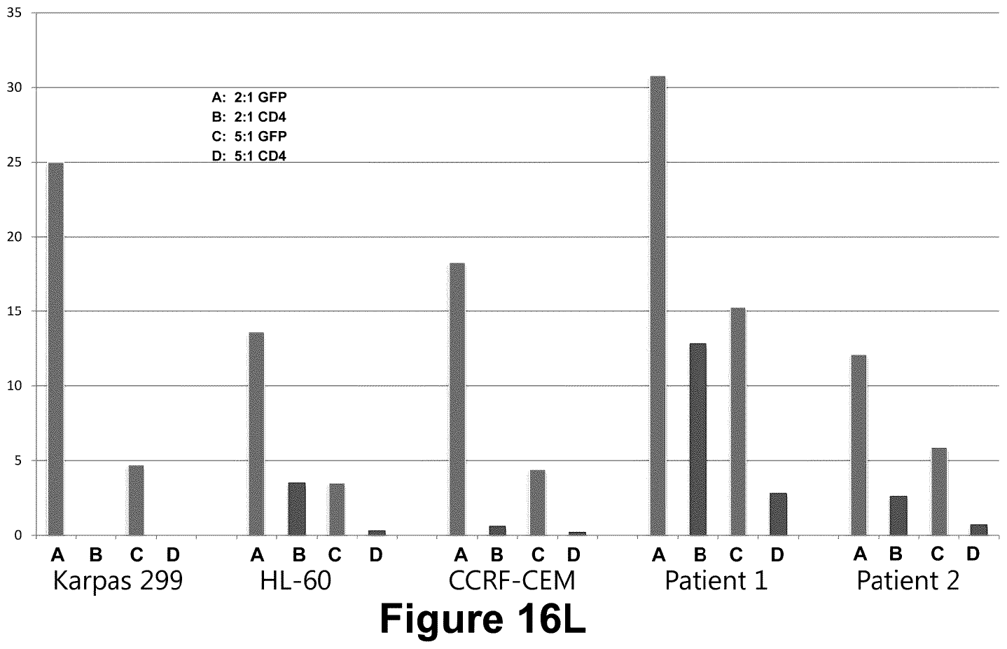

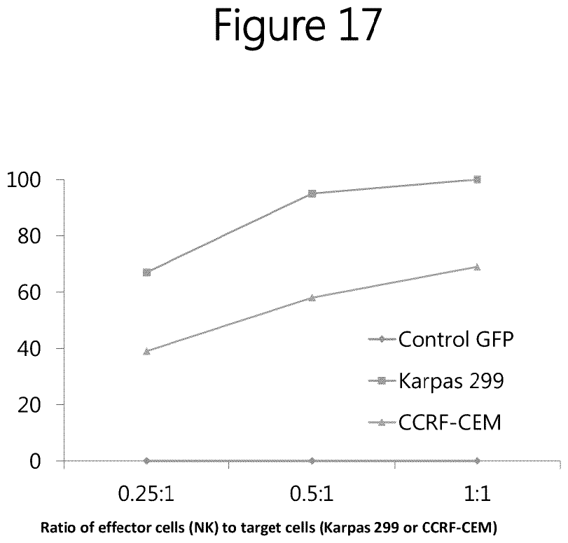

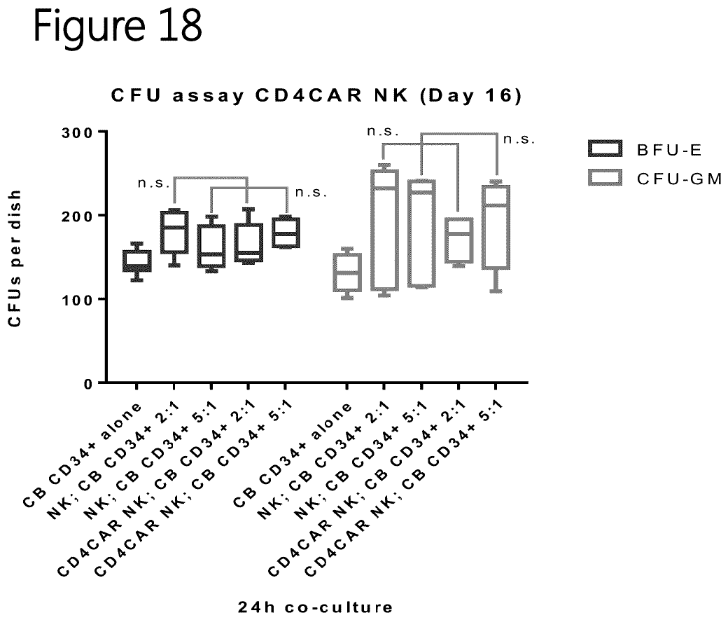

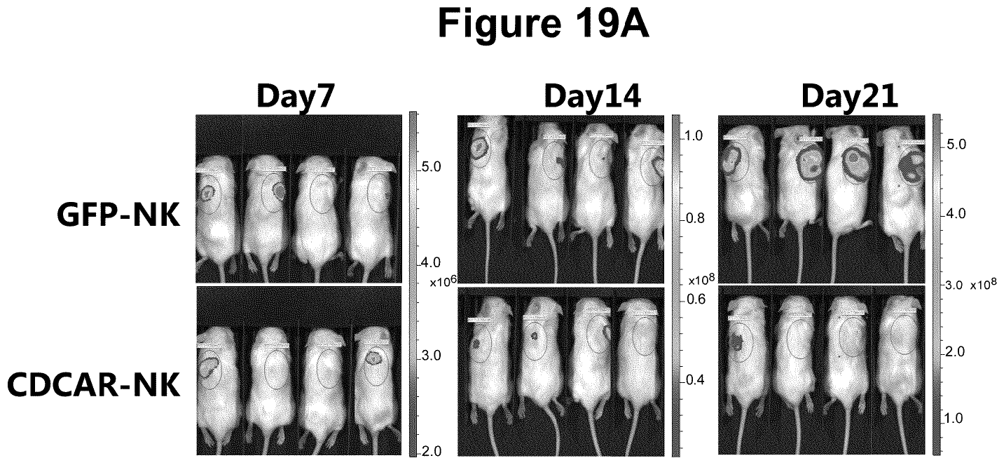

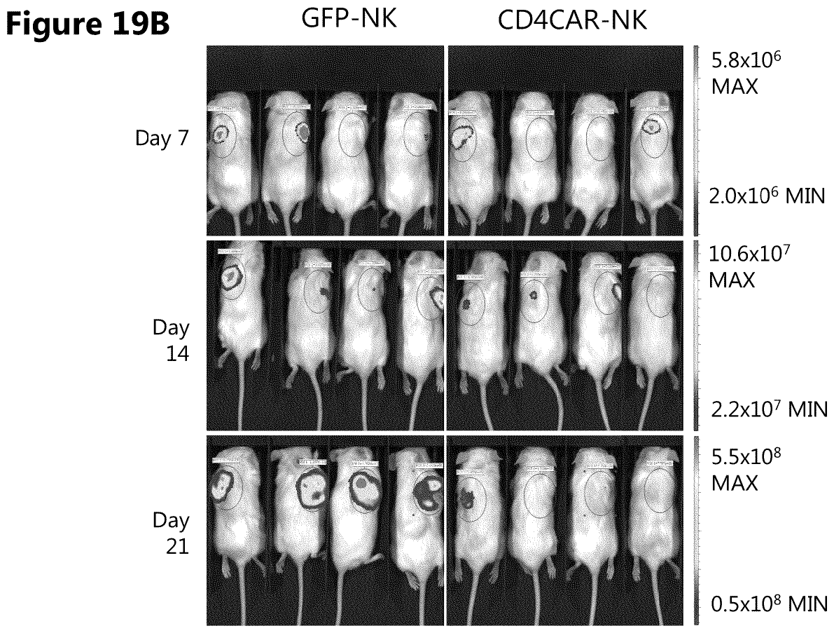

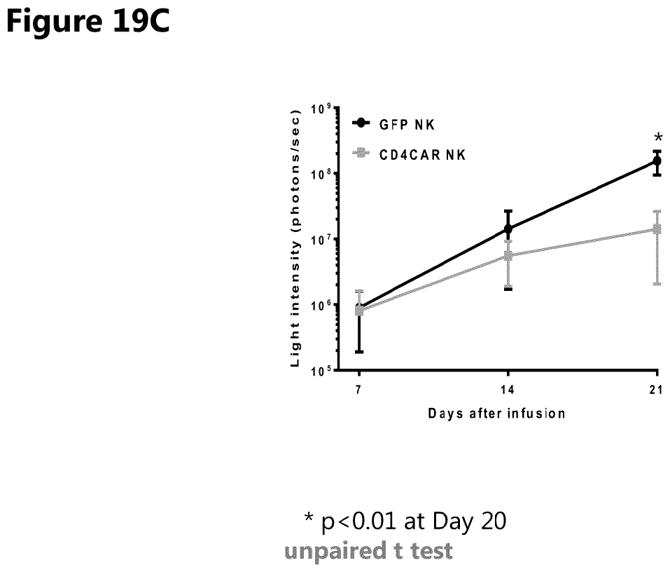

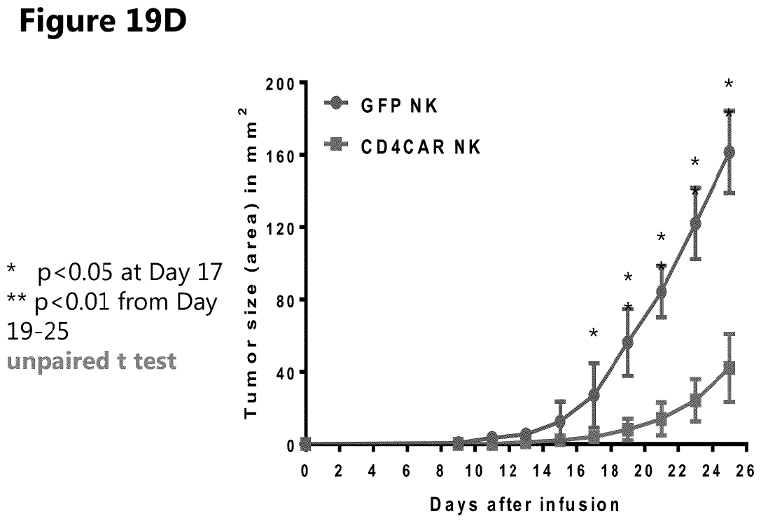

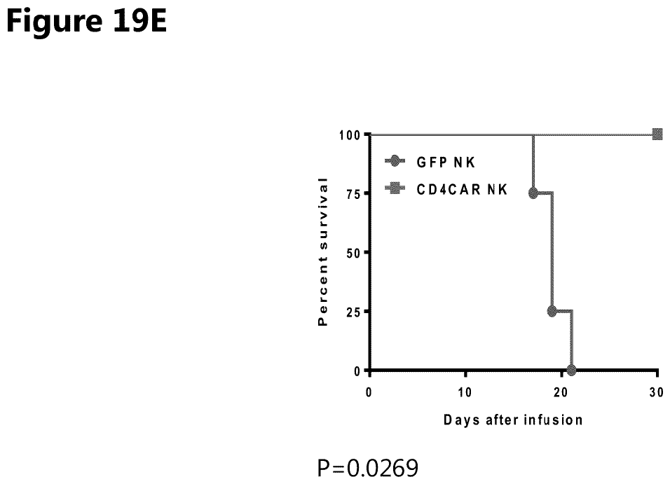

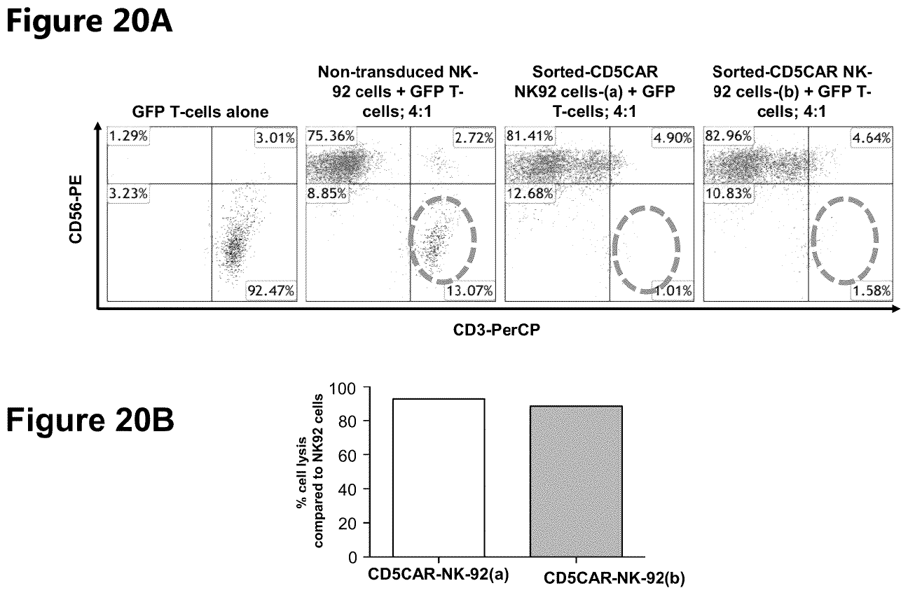

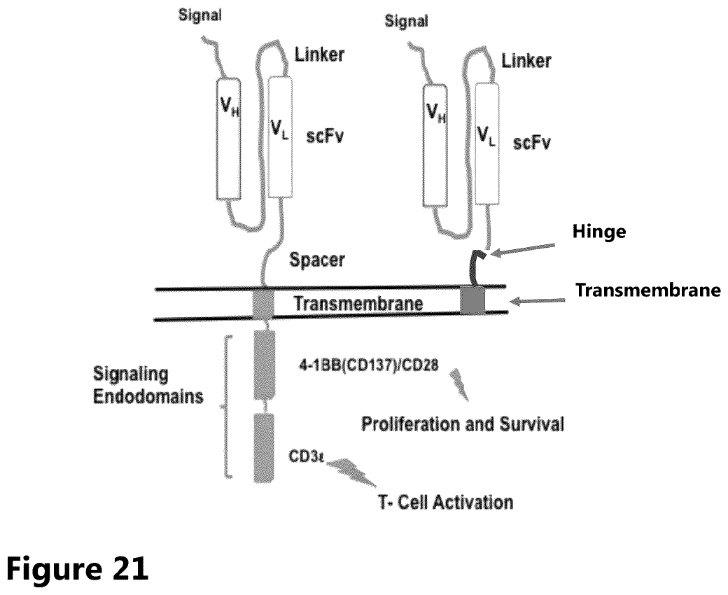

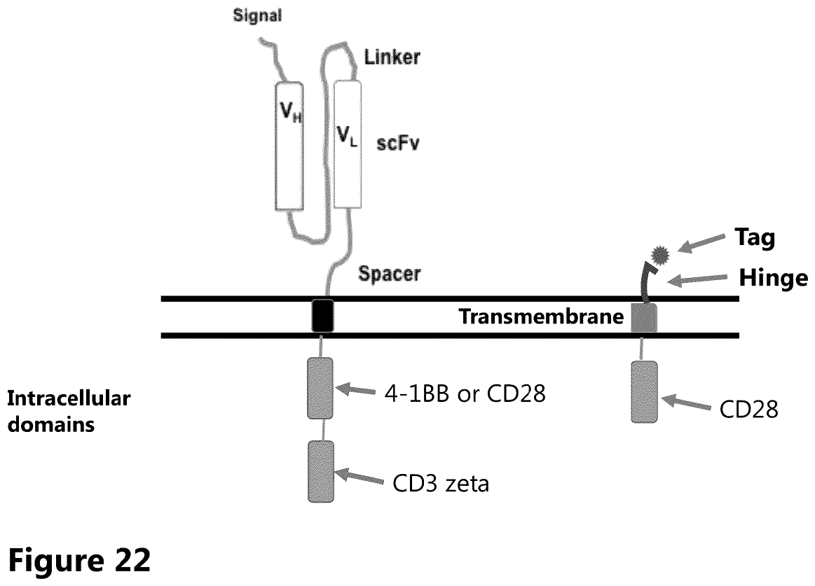

Down-regulation of the CD5 expression on the CAR T cells would also be to simply transduce the T cell population with CDSCAR, then allowing for the population of CDSCAR cells to expand and eliminate all CD5+ T cells within the population, resulting in a CD5 negative CDSCAR T-cell population without the need for CD5 knockdown using CRISPR/Cas9 in situ genetic editing. T cells were transduced with a lentivirus containing the CDSCAR genetic construct (FIG. 1A) with an either single or double transduction technique (FIGS. 2A and 2B). Complete CD5 protein down-regulation was observed only with the double transduction technique after a 4-day incubation (FIG. 2C). The elimination of surface CD5 expression on the doubly transduced T cell population remained stable until incubation day 8.

In order to further elucidate the mechanism by which CDSCAR down-regulates CD5 expression on T cells, a new construct was created entitled anchored CD5 scFv (FIG. 1A). This construct comprises an anti-CD5 scFv lined to a transmembrane domain via a hinge region, which allows CD5 scFv to anchor on the T cell surface. The anchored CD5 scFv binds to CD5 target without target cell lysis as observed with a functional CDSCAR. A single transduction and flow data analysis is shown in FIGS. 3A and 3B, with partial down-regulation of CD5 expression for T cells on day 7 of incubation. This is consistent with the partial down-regulation of CD5 expression seen for CDSCAR T-cells after a single transduction.

### CD5CAR Cytotoxic Studies

The successful transduction of CDSCAR was demonstrated utilizing HEK293 cells and T cells. CDSCAR protein expression in HEK293 cells was confirmed by western blot analysis (FIG. 1B). Flow cytometry analysis confirmed CDSCAR expression in transduced T-cells (FIG. 1C). Once the CDSCAR constructs were generated, anti-CD5-directed cytotoxicity was tested via CDSCAR T cell co-culture with T-ALL cell lines and patient samples. Co-culture CDSCAR T cell “killing efficiency” was assessed via flow cytometric analysis.

### CD5CAR T Cells Bear the Potent Target Cell Killing Ability

The killing ability of CDSCAR T cells was first tested against T-cell ALL established cell lines CCRF-CEM and Molt-4, and an anaplastic large cell leukemic cell line KARPAS 299 as shown in FIGS. 5A and 5B. An avid killing ability was seen for the two CD5+ cell lines when compared to GFP control, with target cell lysis above 75% for both lines.

The CDSCAR ability to lyse patient sample T-ALL cells was also assessed using multiple patient samples and CDSCAR cell co-cultures as shown in FIG. 6 and FIG. 7. While there was an avid cell killing noted for the T-ALL 1 patient leukemic cells that was similar to the CD5 target cell lysis seen when CDSCAR cells targeted T cell ALL cell lines, three other patient leukemic cells showed comparatively weaker lysis of target cells (FIG. 6A. and FIG. 6B).

The ability of killing by CDSCAR on the patient leukemic cells correlated with the intensity of CD5 expression as shown in FIGS. 6A, 6B, and 6D. As shown in FIGS. 6C and 6D, the CD5 expression for T-ALL-1, T-ALL 3, T-ALL 6 and T-ALL 7 through flow cytometry analysis was observed. The CD5 expression was significantly lower for the T-ALL patient samples, except for T-ALL-1 sample.

### CDSCAR T Cells Exhibits the Specificity and Potent Target Cell Killing

As a control, the CDSCAR T cells were also tested for their ability to ablate CD5 negative leukemic T cells. Anaplastic large T cell lymphoma line is the cell line that does not express CD5. Flow cytometry analysis showed that CDSCAR T cells were unable to lyse or eliminate KARPAS 299 cells, as shown in FIG. 5A, lower panel.

A patient sample (T-ALL-8) with a high level of CD5 expression was obtained from a patient with a minimal disease of T-ALL. Co-culture was performed with CDSCAR and analyzed in detail as shown in FIG. 7. Three population cells including CD5+ normal T cells, CD5+CD34+T-ALL cells and CD5-CD34+T-ALL cells were assessed by flow cytometry after co-culture. CDSCAR exhibited the specificity and potent target cell lysis ability with >95% of CD5 positive cell lysis for all CD5+ cell populations when compared to GFP control. The CDSCAR killed leukemic cells as efficiently as CD5 normal T cells. Killing was not observed in the CD5-34+ leukemic population.

### CD123CAR NK Cells

### Generation of the Third Generation CD123CAR

CD123 CAR construct comprised of leader sequence, anti-CD123 scFv (single-chain fragment), a hinge (H) region, a transmembrane domain (TM), co-stimulatory domains (CD28, 4-1BB), and an intracellular signaling domain, CD3 zeta chain (FIG. 8A). Since there were two co-activation domains (CD28 and 4-1BB) in CD123CAR, it was considered as a third generation CAR. The CD123CAR expression was carried by a strong promoter, SFFV.

### Characterization of CD123CAR

To verify the CD123CAR construct, transfected HEK 293 cells were subjected to Western blot analysis. Immunoblotting with an anti-CD3zeta monoclonal antibody showed a band of predicted size for the CD123CAR CD3zeta fusion protein as shown in FIG. 8B. There was no band for the GFP control vector as shown in FIG. 8B. In addition, NK-92 cells were transduced with either CD123CAR or GFP control lentiviruses twice in the course of 3 days, and then washed with fresh culture media. Transduced NK-92 cells were expanded for 2 or 3 days, expanded cells were labeled to determine transduction efficiency as shown in FIGS. 9A and 9). The transduced cells were analyzed by flow cytometry and approximately about 35% of NK-92 cells expressed CD123CAR as shown in FIGS. 9A and 9B. The transduced NK-92 cells were also sorted after expansion resulting in up to about 94% cells expressing CD123CAR as shown in FIG. 9C.

### CD123CAR NK Cells Effectively Lysed Leukemic Cells in Co-Culture Assay

In order to test the function of CD123CAR NK cells, CD123CAR NK cells were cultured with AML cell lines that expressed CD123. In this case, an AML cell line KG1A, which expressed about 72% of CD123, was used in this co-culture experiment as shown in FIG. 10A. Three different ratios were used: 0.5 to 1, 1 to 1, and 2 to 1 (with increasing number of CD123CAR NK cells). After 3 or 4h of incubation, effective lysis by CD123CAR NK cells was observed as compared to GFP control in a dose-dependent manner as shown in FIG. 10B. A similar observation was seen in another AML cell line, TF1 as shown in FIG. 10C. FIG. 10D shows the linear graph of the killing percentage of CD123 positive population in these AML cell lines in a dose-dependent manner.

### CD123CAR NK Cells Effectively Lysed Human AML Cells

The killing ability of CD123CARNK cells using patient AML cells (AML-9) was performed using an approach similar to co-culture assay described above. Both CD34 and CD123 were expressed in human sample shown in FIG. 11A. CD123CAR NK cells or GFP-transduced NK cells were used to co-culture with patient's leukemic cells at the ratio of 5 to 1 for 3h incubation (5 CD123CAR NK cells to 1 target cell). The effective lysis by CD123CAR NK cells as compared to that of control, GFP cells was observed. The graph shows a high percentage of killing in both CD123 and CD34 positive leukemic populations as shown in FIG. 11C. These studies support the notion that the CD123CAR NK cells can be used to kill or eliminate CD123 positive leukemic or normal cells in vitro or in vivo.

### CD3CAR NK Cells

### Characterization of CD3CAR NK Cells

A third generation of CD3CAR was generated comprising leader sequence, anti-CD3 scFv (single-chain fragment), a hinge (H) region, a transmembrane domain (TM), co-stimulatory domains (CD28, 4-1BB), and an intracellular signaling domain, CD3 zeta chain. A strong promoter, SFFV, controls the CD3CAR expression. CD3CAR lentiviruses were generated using a similar method described above. Lenti-CD3CAR viruses were used to transduce NK-92 cells. The resulting NK-cells were called as CD3CAR NK cells. The lysis property of CD3CAR NK cells was characterized by testing on the normal T cell population expressing CD3 in the co-culture assay. Co-culture conditions were carried out in NK cell media with 2.5% serum. Co-cultures were incubated for 4 hours and labeled for flow cytometry analysis. The ability of CD3-CAR NK cells to lyse target CD3 cells was evaluated by comparing the amount of residual CD3+ GFP T-cells after co-culture. CD3CAR NK cells exhibited potent target cell lysis with killing of greater than about 60% of CD3 positive T cells at the ratios of E:T, 2:1 and 5:1 when compared to GPF controls as shown in FIG. 12A. With an increased incubation period to 24h, target CD3+ GFP T-cells were dramatically increased with greater than about 85% killing efficiency at a ratio of 5:1 (effector to target cell) (FIGS. 12A and 12B). These studies indicate that CD3CAR NK cells bear a potent ability of killing CD3 positive cells. The CD3CAR NK cells can be used to kill or eliminate CD3 positive leukemic or normal cells in vitro or in vivo.

In Vitro and In Vivo CD4-Specific Chimeric Antigen Receptor (CAR)-Engineered NK Cells

Chimeric antigen receptor (CAR) immunotherapy has shown exceptional promise in targeting otherwise untreatable hematologic and solid tumor malignancies, providing new hope to both pediatric and adult patients. Although remarkable progress has been achieved in clinical trials for patients with relapsed/refractory B cell malignancies, CAR immunotherapy for patients with T cell leukemias and lymphomas has not yet been developed, despite a generally poorer prognosis stage-for-stage. In light of this unmet clinical need, we engineered natural killer (NK) cells to express a third generation CAR directed against CD4. In contrast to donor T cells, CAR NK cells have the advantage of mediating anti-cancer effects without the risk of inducing graft-versus-host disease (GvHD). Also, their shorter lifespan relative to T cells may limit off-target events and thus eliminate the need for a “suicide switch” that would ablate the modified cells in the event of off-target effects. Other potential advantages of CAR NK cells over CAR T cells include the opportunity to be an off-the-shelf therapy, and simpler manufacturing.

We generated a third generation CD4-specific CAR (CD4CAR) containing CD28, 4-1BB and CD3zeta signaling domains. This CAR was introduced into the NK-92 cell line, which has used in multiple clinical studies, resulting in CD4CAR NK cells. When assayed in co-culture, these CD4CAR cells had a profound ability to kill CD4 positive tumor cells in vitro using both CD4+ cell lines (Karpas 299, CCRF-CEM, and HL60) and primary patient samples from pediatric and adult T cell leukemia and lymphomas. To address any potential CD4CAR NK cell impact on the hematopoietic compartment's ability to repopulate, we also confirmed by CFU assay that CD34+ cells co-cultured with NK CD4CAR cells were able to differentiate into BFU-E and CFU-GM colonies at ratios statistically similar to CD34+ cells co-cultured with non-CAR NK cells. We then confirmed in vivo anti-CD4 positive tumor activity using xenogeneic mouse models. Together, our encouraging results of this preclinical study support the further development of anti-CD4 CAR-engineered NK cell immunotherapy for patients with T cell malignancies.

In our studies, we modified CD8 positive T-cells to express a third generation CD4-specific CAR (CD4CAR) containing CD28, 4-1BB and CD3zeta signaling domains (Pinz et al. 2015). We showed that CD4CAR T-cells have profound ability to kill CD4 positive tumor cells in vitro when co-cultured with Karpas 299 lymphoma cell line as well as primary T-cell leukemia and peripheral T-cell lymphoma (PTCL) cells from two patients. Furthermore, we demonstrated in vivo anti-tumor effects of CD4CAR T-cells using xenogeneic mouse models. Thus, we established the strong therapeutic potential of CD4CAR T-cells in CD4 positive hematologic malignancies.

In this study, we engineered NK-92 cells to express the same third generation CD4CAR and showed that these cells effectively target CD4 positive hematologic malignancies. In our study, CD4CAR NK-92 cells exhibit robust anti-CD4 positive tumor cell activity in vitro against both adult and pediatric lymphoma/leukemia cell lines, CD4 positive T cells isolated from umbilical cord blood, as well as both adult and pediatric T-cell leukemia primary cells. CD4CAR NK-92 cells also present robust in vivo anti-CD4 activity in xenogeneic mouse models. Together, these pre-clinical data support CD4CAR NK cells as a promising bridge to transplant or conditioning regime strategy for the treatment of CD4 positive malignancies that would allow patients with no therapeutic options to qualify for curative bone marrow transplantation.

### Materials and Methods

### Primary Tumor Cells and Cell Lines

Human leukemia cells were obtained from residual samples on a protocol approved by the Institutional Review Board of Stony Brook University. Cord blood cells were also obtained under protocol from donors at Stony Brook University Hospital. Written, informed consent was obtained from all donors. Karpas 299, HL-60, CCRF-CEM, and NK-92 cell lines were from ATCC (Manassas, VA).

### CAR Construct Generation

The CD-4 specific CAR (pRSC.SFFV.CD4.3G) was designed to contain an intracellular CD28 domain upstream of 4-1BB and CD3zeta domains, thereby making the construct a third generation CAR.

### Lentivirus Production and Transduction

To produce viral supernatant, 293FT-cells were co-transfected with pMD2G and pSPAX viral packaging plasmids containing either pRSC.SFFV.CD4.3G or GFP lentiviral vector, using Lipofectamine 2000 (Life Technologies, Carlsbad, CA) according to the manufacturer's protocol.

NK cells were activated for 2 days in the presence of 300 IU/mL IL-2 and 1 ug/mL anti-human CD3 (Miltenyi Biotec, Bergisch Gladbach, Germany) prior to transduction with viral supernatant. Transfection and transduction procedures are further detailed in Supplementary Information.

### CAR Detection on Transduced NK Cells

NK cells were washed and suspended in FACs buffer (0.2% BSA in DPBS) 3 days after the second transduction. To determine CAR expression, flow cytometry analysis was used. Normal goat IgG (Jackson Immunoresearch, West Grove, PA) was used to block nonspecific binding. Each NK cell sample was probed with Biotin-labeled polyclonal goat anti-mouse F(Ab′) 2 (1:250, Jackson) for 30 minutes at 4° C. Cells were washed once, and suspended in FACs buffer. Cells were then stained with PE-labeled streptavidin (1:250, Jackson) for 30 minutes at 4° C. Cells were washed with FACs buffer, and suspended in 2% formalin. Flow cytometry analysis was performed using a FACS Calibur instrument (Becton Dickinson).

### Co-Culture Assays

CD4CAR or GFP (control) NK cells were incubated with CD4 expressing Karpas 299 cells (large T-cell lymphoma), HL-60 cells (acute promyelocytic leukemia), CCRF-CEM cells (T-cell acute lymphoblastic leukemia, or ALL), CD4 positive T cells isolated from human cord blood, or CD4 expressing primary human leukemic cells (adult Sézary syndrome and pediatric T-cell ALL) at ratios of 2:1 and 5:1 (200,000 and 500,000 effector cells to 100,000 target cells, respectively) in 1 mL T-cell culture media, without IL-2. After 24 hours of co-culture, remaining live cells were harvested and stained with mouse anti-human CD56 and CD4 antibodies, and were incubated at 4° C. for 30 minutes. All cells were washed with FACs buffer, suspended in 2% formalin, and analyzed by flow cytometry.

### Co-Culture Killing Curve

CD4CAR or GFP NK cells were incubated with CFSE-stained Karpas 299 cells and CMTMR-stained CCRF-CEM cells at 2:1, 5:1, and 10:1 ratios in 1 mL T-cell culture media, without IL-2. After 24 hours, dead cells were stained with 7-AAD (BioLegend, San Diego, CA). Co-culture cells were then washed with FACs buffer and analyzed by flow cytometry.

### Colony Forming Unit (CFU) Assay

CD4CAR NK cells were incubated at co-culture effector:target ratios of 2:1 and 5:1 respectively with 500 CD34+CB cells for 24 hours in NK cell media supplemented with IL-2. Experimental controls used were CD34+ cells alone, and non-transduced NK cells co-cultured at respective 2:1 and 5:1 effector:target ratios with CD34+CB cells. Hematopoietic compartment output was assessed via formation of erythroid burst-forming units (BFU-E) and number of granulocyte/monocyte colony-forming units (CFU-GM) at Day 16. CFU statistical analysis was performed via 2-way ANOVA with alpha set at 0.05. Day 10 and Day 14 data are included under Supplementary information.

### Reduction of Tumor Burden in NSG Mice by CD4CAR NK Cells

Twelve male 12-week-old NSG mice (NOD.Cg-Prkdcsid Il2rgtm1Wjl/SzJ) were purchased from the Jackson Laboratory and used under a Stony Brook University IACUC-approved protocol. NSG mice were irradiated with a sublethal (2.5 Gy) dose of gamma irradiation. Twenty-four hours later, mice were intradermally injected with 0.5×10⁶ Karpas 299 cells that had been stably transduced to express luciferase, in order to cause a measurable subcutaneous tumor to form. On day 1, twenty-four hours following Karpas 299 cell injection, mice were intravenously injected via tail vein with 5×10 6 CD4CAR NK cells or GFP NK control cells (6 mice per group). Intravenous injections were repeated every 5 days for a total of 6 courses. Tumor size area was measured every other day. On days 7, 14, and 21 following Karpas 299 cell injection, mice were injected subcutaneously with 100 uL RediJect D-Luciferin™ (Perkin Elmer, Waltham, MA) and subjected to IVIS imaging. Images were analyzed using Caliper Life Sciences software (PerkinElmer, Waltham, MA).

### Results

### Generation of the Third Generation CD4CAR

The single-chain variable fragment (scFv) nucleotide sequence of the anti-CD4 molecule was derived from the humanized monoclonal antibody ibalizumab (Hu5A8 or TNX-355)—the safety and efficacy of which have been well studied in clinical trials for HIV (Kuritzkes, Jacobson et al. 2004, Jacobson, Kuritzkes et al. 2009). To improve signal transduction, the CD4CAR was designed with CD28 and 4-1BB domains fused to the CD3 zeta signaling domain, making it a third generation CAR (FIG. 14A). For efficient expression of the CD4CAR molecule on the NK cell surface, a strong spleen focus-forming virus promoter (SFFV) was used and the leader sequence of CD8 was incorporated in the construct. The anti-CD4 scFv was separated from the intracellular signaling domains by CD-8 derived hinge (H) and transmembrane (TM) regions (FIGS. 14A and 14C). The CD4CAR DNA molecule was sub-cloned into a lentiviral plasmid.

### Characterization of CD4CAR

In order to verify the CD4CAR construct, HEK293-FT cells were transfected with the CD4CAR lentiviral plasmid or GFP control plasmid, and 48 hours later were harvested for Western blot analysis. Immunoblotting with an anti-CD3zeta monoclonal antibody showed bands of predicted size for the CD4CAR-CD3zeta fusion protein (FIG. 14B). As expected, no CD3zeta expression was observed for the GFP control protein (FIG. 14B).

### Generation of CD4CAR NK Cells

NK-92 cells were activated and transduced with CD4CAR and GFP control lentiviral constructs. NK cells were activated for 2 days with an anti-CD3 antibody and cultured in the presence of IL-2. Cells were transduced with either CD4CAR or GFP (See FIG. 15A). CD4CAR NK transduction efficiency was determined to be 15.9%, as determined by flow cytometry (FIG. 15B). Next, fluorescence-activated cell sorting (FACS) was used in order to further enrich for CD4CAR positive NK cells. Following sorting, collected CD4CAR^{high} NK cells were confirmed to be more than 85% CD4CAR positive (Supplementary FIG. 15). After FACS collection of CD4CAR^{high} cells, CD4CAR expression levels remained consistently stable at 75-90% on NK cells during expansion of up to 10 passages, and following cryofreezing. Indeed, at the onset of co-culture experiments, expanded CD4CAR^{high} NK cells still expressed CAR at 85%.

### (FIG. 15C).

### CD4CAR NK Cells Specifically Kill CD4 Positive Tumor Cells

CD4CAR NK cells were then tested for anti-lymphoma activity in vitro using the following CD4 positive cell lines: Karpas 299, HL-60, and CCRF-CEM. The Karpas 299 cell line was established from the peripheral blood of a 25-year-old patient with large T-cell lymphoma. The HL-60 cell line was established from the peripheral blood of a 36-year-old patient with acute promyelocytic leukemia. The CCRF-CEM cell line was established from the peripheral blood of a 4-year-old patient with acute lymphoblastic leukemia (ALL).

During 24-hour co-culture experiments, CD4CAR NK cells showed profound killing of CD4 positive leukemia/lymphoma established cell line cells at both effector cell to target cell ratios (E:T) of 2:1 (FIG. 16A, 16C, 16E) and 5:1 (FIG. 16B, 16D, 16F). As expected, control GFP NK cells showed some non-specific tumor cell killing ability that is innate to NK cells, but were not as effective against CD4 positive tumor cells as CD4CAR NK cells were. At the E:T ratio of 2:1, CD4CAR NK cells successfully eliminated Karpas 299 cells (0.0% CD4 positive cells remaining), while GFP NK cells did not (25% target cells remaining) (FIG. 16A). Analysis of Karpas 299 cells alone confirmed high expression of CD4 on this cell line (99.1%) (FIG. 16A, 16B). Similarly, analysis of HL-60 and CCRF-CEM cells alone confirmed high expression of CD4 (99.9% and 92.1%, respectively) (FIGS. 16C-16F). Accordingly, at a 2:1 E:T, CD4CAR NK cells also killed HL-60 cells and CCRF-CEM cells (3.5% and 0.6% CD4 positive cells remaining, respectively), while GFP NK cells did not (13.6% and 18.3% target cells remaining, respectively) (FIGS. 16C-16F). These data show that CD4CAR NK cells specifically target CD4 positive cells in addition to retaining non-specific anti-tumor cell activity intrinsic to NK cells.

Co-culture studies were also conducted using patient samples (FIGS. 16G-16J). Patient 1 presented with Sézary syndrome, an aggressive form of CD4 positive T-cell leukemia that did not respond to standard chemotherapy (FIGS. 16G, and 16H). Patient 2 presented with pediatric T-cell ALL (FIGS. 16I, 16J). Flow cytometry analysis of both patient samples incubated alone showed that 78.1% and 43.7% of leukemia cells expressed CD4. At E:T of 2:1, CD4CAR NK cells co-cultured for 24 hours with leukemic cells from patients 1 and 2 showed that of the remaining cells, only 12.8% and 2.6% were CD4 positive, respectively. At an E:T of 5:1, anti-tumor cell activity was further evident with only 2.8% and 0.7% CD4 positive cells remaining. When co-cultures were performed with control GFP NK cells and compared to those with CD4CAR NK cells, a greater percentage of remaining cells were CD4 positive for both E:T evaluated and for both patient samples used. Therefore, we showed that in co-culture assay, CD4CAR NK cells successfully targeted both adult and pediatric CD4 positive and aggressive leukemia.

Additional co-culture studies were conducted using CD4 positive T cells isolated from cord blood. In these experiments, CD4CAR NK cells depleted CD4 positive T cells at effector:target ratio of 2:1 after 24 hours of co-culture (0.0% CD4 positive cells remaining), but GFP NK cells did not (29.6% CD4 positive cells remaining) (FIG. 16K).

In summary, CD4 positive cells both patient and cell line were lysed when co-cultured with CD4CAR NK cells when compared to GHP NK co-culture (FIG. 16L).

### CD4CAR NK Cells Kill CD4-Expressing Tumor Cell Lines in Dose Dependent Manner

CD4CAR NK cells were shown to specifically kill CD-4 expressing Karpas 299 and CCRF-CEM leukemic cell lines in a dose dependent manor in a co-culture assay evaluating effector:target ratios of 1:4, 1:2, and 1:1 (FIG. 17). CD4CAR NK effector cells or GFP NK effector cells were incubated with CFSE-stained Karpas 299 and CMTMR-stained CCRF-CEM target cells at stated ratios. After 24 hours, 7-AAD dye was added and unstained live cells were analyzed by flow cytometry (FIG. 17). Percent killing of target cells was measured by comparing CD4 positive target cell survival in CD4CAR NK co-culture relative to that in GFP NK control co-culture. Karpas 299 cells were eliminated at rates of 67%, 95%, and 100%, at effector to target ratios of 1:4, 1:2, and 1:1, respectively (FIG. 17). CCRF-CEM cells were eliminated at rates of 39%, 58%, and 69%, at the same E:Ts, respectively (FIG. 17). These data indicate a dose-response relationship for CD4CAR NK cells.

### CD4CAR NK Cells do not Affect Stem Cell Output in Hematopoietic Compartment

CFU (Colony-Forming-Unit) assay analysis revealed that CD4CAR NK cells did not significantly affect the CD34+ Cord Blood (CB) stem cell output of the hematopoietic compartment. Hematopoietic compartment output is assessed by the presence of erythroid progenitors and granulocyte/macrophage progenitors at Day 0, which is measured by number of erythroid burst-forming units (BFU-E) and number of granulocyte/monocyte colony-forming units (CFU-GM) at Day 16 (FIG. 18). This data is consistent with CD4CAR NK cells targeting CD4 specifically, which is a more mature T-cell marker, with limited impact on hematopoietic stem cells and early progenitors, and no evidence of pronounced lineage skewing.

### CD4CAR NK Cells Exhibit Significant Anti-Tumor Activity In Vivo

In order to evaluate the in vivo anti-tumor activity of CD4CAR NK cells, we developed a xenogeneic mouse model using NSG mice sublethally irradiated and intradermally injected with luciferase-expressing Karpas 299 cells to induce measurable tumor formation. On day 1, 24 hours following Karpas 299 cell injection, and every 5 days after for a total of 6 courses, mice were intravenously injected with 5×10⁶ CD4CAR NK cells or GFP NK control cells. On days 7, 14, and 21, mice were injected subcutaneously with RediJect D-Luciferin™ and subjected to IVIS imaging to measure tumor burden (FIGS. 19A and B). Average light intensity measured for the CD4CAR NK injected mice was compared to that of GFP NK injected mice (FIG. 19 C). Unpaired T test analysis showed a significant difference between the two groups by day 21 with less light intensity and thus less tumor burden in the CD4CAR NK injected group than in the GFP NK injected group (p<0.01). On day 1, and every other day after, tumor size area was measured and the average tumor size between the two groups was compared (FIG. 19 D). Unpaired T test analysis showed that the average tumor size of CD4CAR NK injected mice was significantly smaller than that of GFP NK injected mice starting on day 17 (p<0.05) and continuing on days 19-25 (p<0.01). Percent survival of mice was measured and compared between the two groups (FIG. 19E). All of the CD4CAR NK injected mice survived through day 30 (FIG. 19E). However, percent survival of GFP NK injected mice started to decline by day 17 with no survival by day 23 (FIG. 19E). In summary, these in vivo data indicate that CD4CAR NK cells significantly reduce tumor burden and prolong survival in Karpas 299-injected NSG mice when compared to GFP NK control cells.

### CDSCAR NK Cells Exhibits a Potent Killing Ability of CD5 Positive Cells.

CD5 CAR NK-92 were generated by transduction of lenti-CDSCAR viruses. The CDSCAR expression on NK-92 cells was sorted by flow cytometry. The sorted CDSCAR NK-92 cells were used for killing assays.

In order to determine the killing ability of Natural Killer cell derived CDSCAR cells (CDSCAR NK cells), we completed a co-culture experiment and flow data analysis. We compared GFP transduced T cells alone (the most left pane) to non-transduced NK-92 cells co-cultured with GFP labeled T-cells and CDSCAR NK-92 cells co-cultured with GFP labeled T-cells (FIG. 20). The NK-92 cells were transduced with lenti-CDSCAR viruses. The transduced cells, CDSNK-92 cells were sorted by flow cytometry. The CD5 positive T cells were labeled with GFP with lenti-GFP viruses. All effector:target ratios were 4:1, and all co-cultures were performed for a duration of 4 hours. GFP-transduced T-cells were detected by CD3-PerCP antibody and NK-92 cells were identified CD56-PE antibody. The % of cell lysis compared to non-transduced NK92, both of sorted CD5CARNK-92 cells (preparation a, and preparation b) showed over 88% of cell lysis activity against T cells (FIG. 20A).

A bar graph (FIG. 20B) summarizes the results seen with flow cytometry analysis (FIG. 20A). This bar graph indicates % of cell lysis activity by sorted CDSCAR NK-92-(a) or -(b) cells compared to the non-transduced NK92 cells in co-culture assay described in FIG. 20A. CDSCAR NK-92 cells almost entirely eliminate CD5 positive cells in a co-culture assay.

### Discussion

The efficacy of CAR NK cells has been well-established in pre-clinical studies using both primary human NK cells as well as the NK-92 cell line used in this study (Glienke, Esser et al. 2015). Clinical validation on efficacy of CAR NK cells is currently underway in two ongoing studies using CD-19 CAR modified donor-derived or haploidentical NK cells in patients with B-cell ALL (NCT00995137 and NTC01974479).

NK cells are unique effector cells in that they possess innate tumor associated antigen independent cytotoxicity via multiple natural cytotoxicity receptors. Additionally, they have the capacity for antibody-dependent cell-mediate cytotoxicity as they express Fc fragment of IgG, low affinity III receptor (FcRYIII). Therefore, when compared to CAR T cells, CAR NK cells have the advantage of targeting tumor cells via multiple mechanisms. Additionally, the cytokine production profile of NK cells is less pro-inflammatory when compared to those of T cells. Furthermore, NK cells have been shown to serially kill tumor cells.

Finally, in contrast to T cells, NK cells have a relatively short lifespan of approximately 2 weeks. Therefore, it is expected that NK cells would be exhausted shortly after destroying cancer cells. Thus, unlike CAR constructs in T cells, those in NK cells would not necessarily require incorporation of safety modifications such as inducible suicide genes, and long-term toxicity would not be expected.

The short lifespan of NK cells affords CAR NK cells unique clinical applications. For example, CD4CAR NK therapy would be a particularly attractive therapeutic option for CD4 expressing leukemia/lymphoma in patients with minimal disease, resistant to standard chemotherapy. In these cases, CD4CAR NK therapy could be used to specifically target and eliminate cancer cells and fall out of circulation shortly after. Furthermore, there may be no subsequent need for bone marrow transplant/stem cell rescue following CD4 NK therapy, as hematopoietic stem cells do not uniformly express CD4 and myeloablation would not be expected, as supported by the CFU analysis shown in this study. In fact, multiple clinical studies with monoclonal antibody-based therapies have shown that ablation of CD4 expressing cells is well tolerated in patients with T-cell lymphoma without evidence of irreversible immunosuppression or other long-term adverse events (Knox, Hoppe et al. 1996, Hagberg, Pettersson et al. 2005, Kim, Duvic et al. 2007).

On the other hand, CD4CAR NK cells may also be useful as a bridge to bone marrow transplant in candidates who do not meet criteria for transplant due to a small percentage of residual blasts following standard chemotherapy treatment. These potential clinical indications for CD4CAR are particularly significant given the markedly poor prognosis associated with T-cell malignancies.

As our CD4CAR construct was designed based on that of the humanized monoclonal antibody ibalizumab (Hu5A8 or TNX-355), we propose that the in vivo specificity of the CD4CAR should be similar, if not nearly identical, to that of ibalizumab. Clinical studies to date utilizing ibalizumab have already characterized the safety profile and efficacy of this molecule in patients with HIV (Kuritzkes, Jacobson et al. 2004, Jacobson, Kuritzkes et al. 2009), and as mentioned above, other anti-CD4 monoclonal antibody studies have shown that ablation of CD4 positive cells is well tolerated. Additionally, although to our knowledge this is the only CD4CAR studied in hematologic malignancy to date, there have been preclinical studies of a CD4CAR used in T cells to target HIV (Liu, Patel et al. 2015). Thus, based on these studies, our pre-clinical data presented here, we anticipate that our CD4CAR would likely be effective with a tolerable safety profile in patients with CD4 positive malignancies.

The NK-92 cell line used in this study has been shown to pose a low tumorigenicity risk when irradiated and transfused in oncology patients (Tonn, Becker et al. 2001). One safety aspect that supports use of this cell line is that NK-92 cells are dependent on IL-2 for growth and cytotoxic activity. More importantly, studies have shown that irradiation of NK-92 cells halts cell division without diminishing cytotoxicity, and as a result, the safety and efficacy of irradiated NK-92 cells has been well-established oncologic clinical trials (Tonn et al., (2001). “Cellular Immunotherapy of Malignancies Using the Cloncal Natural Killer Cell Line NK-92.” Journal of the Hematotherapy & Stem Cell Research 10: 535-544; Tonn et al. (2013). “Treatment of patients with advanced cancer with the natural killer cell line NK-92.” Cytotherapy 15(12): 1563-1570; Arai, Meagher et al. 2008).

In this study we provide pre-clinical evidence for the efficacy and specificity of CD4CAR NK cells in targeting CD4 expressing leukemia and lymphoma. The strong potential for a favorable safety profile of this novel immunotherapy is supported by the relatively short half-life of NK effector cells, the absence of CD4 on hematopoietic stem cells, and studies with anti-CD4 monoclonal antibodies from which our anti-CD4 CAR construct was designed. Clinical studies of CD4CAR NK cells will need to be carried out to further investigate the efficacy and safety of this novel immunotherapy in CD4 expressing hematologic malignancies.

### Supplementary Information

### Detailed Lentivirus Production and Transduction of NK Cells

To produce viral supernatant, 293FT-cells were co-transfected with pMD2G and pSPAX viral packaging plasmids containing either pRSC.SFFV.CD4.3G or GFP lentiviral vector, using Lipofectamine 2000 (Life Technologies, Carlsbad, CA) according to the manufacturer's protocol, and incubated for 6 hours. Cells were then washed and suspended in DMEM with 10% FBS, sodium butyrate, sodium pyruvate, and HEPES (20 mM) (all Gibco). Viral supernatant was collected 24 and 48 hours after transfection, cleared of cellular debris via centrifugation and filtration (0.45 uM), aliquoted, and flash frozen in liquid nitrogen for storage at −80° C.

To confirm virus production, 293-FT cells were harvested 48 hours after transfection, lysed in 1 mL RIPA buffer with deoxycholate and protease inhibitor cocktail (June, Linette et al. 1993), and 10 uL sample was electrophoresed on a 10% PAGE-SDS gel, and transferred to Immobilon FL (0.45 uM) membrane using the wet cell method. Milk (5%) in TBS/Tween was used to block blots. Blots were probed with anti-CD247/CD3z (Thermo Fisher™ Holtsvile, NY) at 1:500 overnight, washed 4 times with TBS/Tween, and probed with anti-goat IgG, HRP-conjugated antibody (Thermo Fisher™) at 1:5000 for 2 hours. Following additional washes, HRP substrate (HyGlow, Denville, Holliston, MA) was added to the membrane and the membrane was exposed to autoradiographic film.

NK-92 cells (ATCC; Manassas, VA) were activated for 2 days in the presence of 300 IU/mL IL-2 and 1 ug/mL anti-human CD3 (Miltenyi Biotec, Bergisch, Gladbach, Germany). A non-tissue culture treated 6-well plate was coated with RetroNectin™ (Clontech, Mountain View, CA) at 15 ug/mL in DPBS for 2 hours at room temperature or overnight at 4° C. Wells were blocked with 2% BSA in PBS for 30 minutes at room temperature, then washed once with PBS. Viral supernatant (CD4CAR or GFP) was diluted 1:1 with DMEM containing 10% FBS and added to the washed wells by centrifugation at 2000 g for 2 hours at 32° C. Wells were washed once with NK cell media, and activated NK cells were added, 4 mL per well at 0.5×10⁶ cells/mL, with IL-2 (300 IU/mL. Plates were centrifuged at 1000 g for 10 minutes and incubated overnight at 37° C. in the presence of 5% CO₂. The following morning, a second transduction, identical to the first, was carried out. The morning after that, cells were transferred to a fresh non-coated 6-well plate in NK cell media with IL-2 (300 IU/mL), cells were sorted for CD4CAR+NK cells, and subsequently incubated as above for a total of 7 days from activation.

CFU assays were conducted in 4-7 replicates per set in 35 mm dishes in MethoCult™ H4435 Enriched (Stem Cell Technologies, Vancouver, Canada), optimized for CD34+ purified cord blood. CFU statistical analysis was performed via 2-way ANOVA with alpha set at 0.05.

### CD5CAR NK-92 Cells Almost Eliminate CD5 Positive Cells in a Co-Culture Assay

The NK-92 cells were transduced with lenti-CDSCAR viruses. The transduced cells, CDSNK-92 cells were sorted by flow cytometry. The CD5 positive T cells were labeled with GFP with lenti-GFP viruses. The incubation time for all co-cultures was 4 hrs, with an effector:target cell ratio of 4:1. GFP-transduced T-cells were detected by CD3-PerCP antibody and NK-92 cells were identified CD56-PE antibody. The % of cell lysis compared to non-transduced NK92 (control), both of sorted CD5CARNK-92 cells (preparation a and preparation b) showed over 88% of cell lysis activity against T cells (FIG. 20A)

The results from 20A were summarized in the bar graph (FIG. 20B). This bar graph indicates % of cell lysis activity by sorted CDSCAR NK-92-(a) or -(b) cells compared to the non-transduced NK92 cells in co-culture assay described in above. CDSCAR NK-92 cells almost eliminated CD5 positive T cells in a co-culture assay.

While there have been described what are presently believed to be the preferred embodiments of the present disclosure, those skilled in the art will realize that other and further changes and modifications may be made thereto without departing from the spirit of the disclosure, and it is intended to claim all such modifications and changes as come within the true scope of the disclosure.

1. An engineered cell comprising:
a first polypeptide comprising a chimeric antigen receptor polypeptide; said chimeric antigen receptor polypeptide comprising a first antigen recognition 5 domain, a first signal peptide, a first hinge region, a first transmembrane domain, a co-stimulatory domain, and a signaling domain; and
a second polypeptide comprising a second antigen recognition domain, a second
signal peptide, a second hinge region, and a second transmembrane domain, wherein the second polypeptide does not comprise a co-stimulatory domain or a signaling domain.

2. The engineered cell according to claim 1, wherein the engineered cell is CD5 deficient.

3. The engineered cell according to claim 1, wherein the engineered cell further comprises a third polypeptide comprising CD5 antigen recognition domain.

4. The engineered cell according to claim 3, wherein the CD5 antigen recognition domain comprises an antibody, binding portion or variable region of a monoclonal antibody, or scFv.

5. The engineered cell according to claim 1, wherein said first antigen recognition domain and second antigen recognition domain independently comprise a CD2, CD3, CD4, CD5, CD7, CD8, CD19, CD20, CD22, CD30, CD33, CD52, CD123, CS1, BAFF, TACI, or BCMA antigen recognition domain.

6. The engineered cell according to claim 1, wherein the first antigen recognition domain and the second antigen recognition domain are different.

7. The engineered cell according to claim 1, wherein the first signal peptide and second signal peptide independently comprise CD8, CD45, or CSF.

8. The engineered cell according to claim 1, wherein the first hinge region and second hinge region independently comprise the hinge region from CD8a, CD4, IgG1, IgG2, IgG3, IgG4, or IgD innate immune cells comprising at least one of CD2, CD3, CD4, CD5, CD7, or CD8 are recruited to cancer cells.

9. The engineered cell according to claim 1, wherein the first hinge region and the second hinge region are different.

10. The engineered cell according to claim 1, wherein the first transmembrane domain and second transmembrane domain independently comprise CD3 epsilon, CD4, CD5, CD7, CD8, CD9, CD16, CD22, CD28, CD33, 5 CD41, CD64, CD68, CD86, CD137, or CD154.

11. The engineered cell according to claim 1, wherein the first transmembrane domain and the second transmembrane domain are different.

12. The engineered cell according to claim 1, wherein the engineered cell 10 comprises a T-cell or Natural killer cell.

13. The engineered cell according to claim 1, wherein the engineered cell is CD2, CD3, CD4, CD5, CD7, or CD8 deficient.

14. The engineered cell according to claim 1, wherein the engineered cell comprises recombinant IL-15, IL-15 α, or IL-12.

15. A method of reducing cancer cell proliferation or increasing cancer cell death comprising administering an engineered cell comprising:
a first polypeptide comprising a chimeric antigen receptor polypeptide; said chimeric antigen receptor polypeptide comprising a first antigen recognition domain, a first signal peptide, a first hinge region, a first transmembrane domain, a co-stimulatory domain, and a signaling domain; and
a second polypeptide comprising a second antigen recognition domain, a second signal peptide, a second hinge region, and a second transmembrane domain, wherein the second polypeptide does not comprise a co-stimulatory domain or a signaling domain to a subject in need thereof; and wherein the second antigen recognition domain comprises CD2, CD3, CD4, CD5, CD7, or CD8; and innate immune cells comprising at least one of CD2, CD3, CD4, CD5, CD7, or CD8 are recruited to cancer cells.

16. The method of treating a cell proliferative disease according to claim 15, wherein the cell proliferative disease comprises neuroblastoma, small cell lung cancer, melanoma, ovarian cancer, renal cell carcinoma, colon cancer, lymphoma, childhood acute lymphoblastic leukemia, T cell acute lymphoblastic leukemia, blood cancer, T cell lymphoma, T cell leukemia, precursor acute T cell lymphoblastic leukemia, precursor acute T cell lymphoblastic lymphoma, mantle cell lymphoma, acute myeloid leukemia (AML), B-cell acute lymphoblastic leukemia (B-ALL), hairy cell leukemia, blastic plasmocytoid dendritic neoplasm, EBV-positive T-cell lymphoproliferative disorders, adult T-cell leukemia, adult T-cell lymphoma, mycosis fungoides, sezary syndrome, primary cutaneous CD30 positive T-cell lymphoproliferative disorders, peripheral T-cell lymphoma, angioimmunoblastic T-cell lymphoma, anaplastic large cell lymphoma, and thymic carcinoma.

17. An engineered cell comprising:
a first polypeptide comprising a chimeric antigen receptor polypeptide; said chimeric antigen receptor polypeptide comprising a first antigen recognition domain, a first signal peptide, a first hinge region, a first transmembrane domain, and one of a first co-stimulatory domain and a first signaling domain; and
a second polypeptide comprising a tag binding domain, a second signal peptide, a second hinge region, and a second transmembrane domain, and a second co-stimulatory domain; wherein the second polypeptide does not comprise a signaling domain.

18. The engineered cell according to claim 17, wherein the tag comprises streptavidin, biotin, HIS, MYC, HA, agarose, V5, Maltose, GST, or GFP.

19. The engineered cell according to claim 17, wherein the engineered cell comprises a T-cell or Natural killer cell.

20. The engineered cell according to claim 17, wherein the first transmembrane domain and the second transmembrane domain are different.


SEQUENCE LISTING (46 sequences)

SEQ ID NO: 1 - DNA, 1596 bp, Artificial Sequence
  Synthetic Sequence
gcgatcgcatggccttaccagtgaccgccttgctcctgccgctggccttgctgctccacgccgccaggccggacatccaggtgacccagagccccagcagcctgagcgccagcctgggcgagagaatcagcctgacctgcagaaccagccaggacatcagcaactacctgaactggttccagcagaagcccgacggcaccttcaagagactgatctacgccaccagcagcctggacagcggcgtgcccaagagattcagcggcagcggcagcggcagcgactacagcctgaccatcagcagcctggagagcgaggacttcgccgactactactgcctgcagtacgccagctaccccttcaccttcggcagcggcaccaagctggagatcaagggaggggggggatccgggggaggaggctccggcggaggcggaagcgaggtgcagctgcaggagagcggccccggcctggtgaagcccagccagaccctgagcctgacctgcagcgtgaccggctacagcatcaccagcggctactactggcactggatcagacagttccccggcaacaagctgcagtggatgggctacatcagctacagcggcttcaccaactacaagaccagcctgatcaacagaatcagcatcacccacgacaccagcgagaaccagttcttcctgaacctgaacagcgtgaccaccgaggacaccgccacctactactgcgccggcgacagaaccggcagctggttcgcctactggggccagggcaccctggtgaccgtgagcgccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgatttaaac

SEQ ID NO: 2 - protein, 526 aa, Artificial Sequence
  Synthetic Sequence
MALPVTALLLPLALLLHAARPDIQVTQSPSSLSASLGERISLTCRTSQDISNYLNWFQQKPDGTFKRLIYATSSLDSGVPKRFSGSGSGSDYSLTISSLESEDFADYYCLQYASYPFTFGSGTKLEIKGGGGSGGGGSGGGGSEVQLQESGPGLVKPSQTLSLTCSVTGYSITSGYYWHWIRQFPGNKLQWMGYISYSGFTNYKTSLINRISITHDTSENQFFLNLNSVTTEDTATYYCAGDRTGSWFAYWGQGTLVTVSATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 3 - DNA, 1009 bp, Artificial Sequence
  Synthetic Sequence
gcgatcgcatggccttaccagtgaccgccttgctcctgccgctggccttgctgctccacgccgccaggccggacatccaggtgacccagagccccagcagcctgagcgccagcctgggcgagagaatcagcctgacctgcagaaccagccaggacatcagcaactacctgaactggttccagcagaagcccgacggcaccttcaagagactgatctacgccaccagcagcctggacagcggcgtgcccaagagattcagcggcagcggcagcggcagcgactacagcctgaccatcagcagcctggagagcgaggacttcgccgactactactgcctgcagtacgccagctaccccttcaccttcggcagcggcaccaagctggagatcaagggaggggggggatccgggggaggaggctccggcggaggcggaagcgaggtgcagctgcaggagagcggccccggcctggtgaagcccagccagaccctgagcctgacctgcagcgtgaccggctacagcatcaccagcggctactactggcactggatcagacagttccccggcaacaagctgcagtggatgggctacatcagctacagcggcttcaccaactacaagaccagcctgatcaacagaatcagcatcacccacgacaccagcgagaaccagttcttcctgaacctgaacagcgtgaccaccgaggacaccgccacctactactgcgccggcgacagaaccggcagctggttcgcctactggggccagggcaccctggtgaccgtgagcgccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgctgagtttaaac

SEQ ID NO: 4 - protein, 330 aa, Artificial Sequence
  Synthetic Sequence
MALPVTALLLPLALLLHAARPDIQVTQSPSSLSASLGERISLTCRTSQDISNYLNWFQQKPDGTFKRLIYATSSLDSGVPKRFSGSGSGSDYSLTISSLESEDFADYYCLQYASYPFTFGSGTKLEIKGGGGSGGGGSGGGGSEVQLQESGPGLVKPSQTLSLTCSVTGYSITSGYYWHWIRQFPGNKLQWMGYISYSGFTNYKTSLINRISITHDTSENQFFLNLNSVTTEDTATYYCAGDRTGSWFAYWGQGTLVTVSATTTPAPRPPTPAPTIASQPLSLRPEACRPAAGGAVHTRGLDFACDIYIWAPLAGTCGVLLLSLVITLYC

SEQ ID NO: 5 - DNA, 1585 bp, Artificial Sequence
  Synthetic Sequence
gcgatcgcatggccttaccagtgaccgccttgctcctgccgctggccttgctgctccacgccgccaggccggacgtgcagatcacccagagccccagctacctggccgccagccccggcgagaccatcaccatcaactgcagagccagcaagagcatcagcaaggacctggcctggtaccaggagaagcccggcaagaccaacaagctgctgatctacagcggcagcaccctgcagagcggcatccccagcagattcagcggcagcggcagcggcaccgacttcaccctgaccatcagcagcctggagcccgaggacttcgccatgtactactgccagcagcacaacaagtacccctacaccttcggcggcggcaccaagctggagatcaagggaggggggggatccgggggaggaggctccggcggaggcggaagccaggtgcagctgcagcagcccggcgccgagctggtgagacccggcgccagcgtgaagctgagctgcaaggccagcggctacaccttcaccagctactggatgaactgggtgaagcagagacccgaccagggcctggagtggatcggcagaatcgacccctacgacagcgagacccactacaaccagaagttcaaggacaaggccatcctgaccgtggacaagagcagcagcaccgcctacatgcagctgagcagcctgaccagcgaggacagcgccgtgtactactgcgccagaggcaactgggacgactactggggccagggcaccaccctgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtttaaac

SEQ ID NO: 6 - protein, 522 aa, Artificial Sequence
  Synthetic Sequence
MALPVTALLLPLALLLHAARPDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKGGGGSGGGGSGGGGSQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - DNA, 1594 bp, Artificial Sequence
  Synthetic Sequence
gcgatcgcatggccttaccagtgaccgccttgctcctgccgctggccttgctgctccacgccgccaggccggacatccagatgacccagagccccagcagcctgagcgccagcgtgggcgacagagtgaccatcacctgcagcgccagcagcagcgtgagctacatgaactggtaccagcagacccccggcaaggcccccaagagatggatctacgacaccagcaagctggccagcggcgtgcccagcagattcagcggcagcggcagcggcaccgactacaccttcaccatcagcagcctgcagcccgaggacatcgccacctactactgccagcagtggagcagcaaccccttcaccttcggccagggcaccaagctgcagatcggcggcggcggcagcggcggcggcggcagcggcggcggcggcagccaggtgcagctggtgcagagcggcggcggcgtggtgcagcccggcagaagcctgagactgagctgcaaggccagcggctacaccttcaccagatacaccatgcactgggtgagacaggcccccggcaagggcctggagtggatcggctacatcaaccccagcagaggctacaccaactacaaccagaaggtgaaggacagattcaccatcagcagagacaacagcaagaacaccgccttcctgcagatggacagcctgagacccgaggacaccggcgtgtacttctgcgccagatactacgacgaccactactgcctggactactggggccagggcacccccgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtttaaac

SEQ ID NO: 8 - protein, 525 aa, Artificial Sequence
  Synthetic Sequence
MALPVTALLLPLALLLHAARPDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQIGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 9 - protein, 877 aa, Artificial Sequence
  Synthetic Sequence
MALPVTALLLPLALLLHAARPDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKGGGGSGGGGSGGGGSQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQIGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSTTTPAPRPPTPAPTIASQPLSLRPEACRPAAGGAVHTRGLDFACDFWVLVVVGGVLACYSLLVTVAFIIFWV

SEQ ID NO: 10 - DNA, 2635 bp, Artificial Sequence
  Synthetic Sequence
atggccctgcccgtgaccgccctgctgctgcccctggccctgctgctgcacgccgccaggcccgacgtgcagatcacccagagccccagctacctggccgccagccccggcgagaccatcaccatcaactgcagggccagcaagagcatcagcaaggacctggcctggtaccaggagaagcccggcaagaccaacaagctgctgatctacagcggcagcaccctgcagagcggcatccccagcaggttcagcggcagcggcagcggcaccgacttcaccctgaccatcagcagcctggagcccgaggacttcgccatgtactactgccagcagcacaacaagtacccctacaccttcggcggcggcaccaagctggagatcaagggcggcggcggcagcggcggcggcggcagcggcggcggcggcagccaggtgcagctgcagcagcccggcgccgagctggtgaggcccggcgccagcgtgaagctgagctgcaaggccagcggctacaccttcaccagctactggatgaactgggtgaagcagaggcccgaccagggcctggagtggatcggcaggatcgacccctacgacagcgagacccactacaaccagaagttcaaggacaaggccatcctgaccgtggacaagagcagcagcaccgcctacatgcagctgagcagcctgaccagcgaggacagcgccgtgtactactgcgccaggggcaactgggacgactactggggccagggcaccaccctgaccgtgagcagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaggagcaagaggagcaggctgctgcacagcgactacatgaacatgacccccaggaggcccggccccaccaggaagcactaccagccctacgccccccccagggacttcgccgcctacaggagcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagccccagaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggggcagcggcgccaccaacttcagcctgctgaagcaggccggcgacgtggaggagaaccccggccccatggccctgcccgtgaccgccctgctgctgcccctggccctgctgctgcacgccgccaggcccgacatccagatgacccagagccccagcagcctgagcgccagcgtgggcgacagggtgaccatcacctgcagcgccagcagcagcgtgagctacatgaactggtaccagcagacccccggcaaggcccccaagaggtggatctacgacaccagcaagctggccagcggcgtgcccagcaggttcagcggcagcggcagcggcaccgactacaccttcaccatcagcagcctgcagcccgaggacatcgccacctactactgccagcagtggagcagcaaccccttcaccttcggccagggcaccaagctgcagatcggcggcggcggcagcggcggcggcggcagcggcggcggcggcagccaggtgcagctggtgcagagcggcggcggcgtggtgcagcccggcaggagcctgaggctgagctgcaaggccagcggctacaccttcaccaggtacaccatgcactgggtgaggcaggcccccggcaagggcctggagtggatcggctacatcaaccccagcaggggctacaccaactacaaccagaaggtgaaggacaggttcaccatcagcagggacaacagcaagaacaccgccttcctgcagatggacagcctgaggcccgaggacaccggcgtgtacttctgcgccaggtactacgacgaccactactgcctggactactggggccagggcacccccgtgaccgtgagcagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacttctgggtgctggtggtggtgggcggcgtgctggcctgctacagcctgctggtgaccgtggccttcatcatcttctgggtgttaa

SEQ ID NO: 11 - protein, 877 aa, Artificial Sequence
  Synthetic Sequence
MALPVTALLLPLALLLHAARPQIVLTQSPAIMSASPGEKVTITCSASSSISYMHWFQQKPGTSPKLWIYTTSNLASGVPARFSGSGSGTSYSLTISRMEAEDAATYYCHQRSTYPLTFGSGTKLELKGGGGSGGGGSGGGGSQVQLQQSGAELAKPGASVKMSCKASGYTFTSYRMHWVKQRPGQGLEWIGYINPSTGYTEYNQKFKDKATLTADKSSSTAYMQLSSLTFEDSAVYYCARGGGVFDYWGQGTTLTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQIGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSTTTPAPRPPTPAPTIASQPLSLRPEACRPAAGGAVHTRGLDFACDFWVLVVVGGVLACYSLLVTVAFIIFWV

SEQ ID NO: 12 - DNA, 2634 bp, Artificial Sequence
  Synthetic Sequence
atggccctgcccgtgaccgccctgctgctgcccctggccctgctgctgcacgccgccaggccccagatcgtgctgacccagagccccgccatcatgagcgccagccccggcgagaaggtgaccatcacctgcagcgccagcagcagcatcagctacatgcactggttccagcagaagcccggcaccagccccaagctgtggatctacaccaccagcaacctggccagcggcgtgcccgccaggttcagcggcagcggcagcggcaccagctacagcctgaccatcagcaggatggaggccgaggacgccgccacctactactgccaccagaggagcacctaccccctgaccttcggcagcggcaccaagctggagctgaagggcggcggcggcagcggcggcggcggcagcggcggcggcggcagccaggtgcagctgcagcagagcggcgccgagctggccaagcccggcgccagcgtgaagatgagctgcaaggccagcggctacaccttcaccagctacaggatgcactgggtgaagcagaggcccggccagggcctggagtggatcggctacatcaaccccagcaccggctacaccgagtacaaccagaagttcaaggacaaggccaccctgaccgccgacaagagcagcagcaccgcctacatgcagctgagcagcctgaccttcgaggacagcgccgtgtactactgcgccaggggcggcggcgtgttcgactactggggccagggcaccaccctgaccgtgagcagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaggagcaagaggagcaggctgctgcacagcgactacatgaacatgacccccaggaggcccggccccaccaggaagcactaccagccctacgccccccccagggacttcgccgcctacaggagcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagccccagaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggggcagcggcgccaccaacttcagcctgctgaagcaggccggcgacgtggaggagaaccccggccccatggccctgcccgtgaccgccctgctgctgcccctggccctgctgctgcacgccgccaggcccgacatccagatgacccagagccccagcagcctgagcgccagcgtgggcgacagggtgaccatcacctgcagcgccagcagcagcgtgagctacatgaactggtaccagcagacccccggcaaggcccccaagaggtggatctacgacaccagcaagctggccagcggcgtgcccagcaggttcagcggcagcggcagcggcaccgactacaccttcaccatcagcagcctgcagcccgaggacatcgccacctactactgccagcagtggagcagcaaccccttcaccttcggccagggcaccaagctgcagatcggcggcggcggcagcggcggcggcggcagcggcggcggcggcagccaggtgcagctggtgcagagcggcggcggcgtggtgcagcccggcaggagcctgaggctgagctgcaaggccagcggctacaccttcaccaggtacaccatgcactgggtgaggcaggcccccggcaagggcctggagtggatcggctacatcaaccccagcaggggctacaccaactacaaccagaaggtgaaggacaggttcaccatcagcagggacaacagcaagaacaccgccttcctgcagatggacagcctgaggcccgaggacaccggcgtgtacttctgcgccaggtactacgacgaccactactgcctggactactggggccagggcacccccgtgaccgtgagcagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacttctgggtgctggtggtggtgggcggcgtgctggcctgctacagcctgctggtgaccgtggccttcatcatcttctgggtgtaa

SEQ ID NO: 13 - protein, 844 aa, Artificial Sequence
  Synthetic Sequence
MALPVTALLLPLALLLHAARPDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKGGGGSGGGGSGGGGSQIQLVQSGPDLKKPGETVKLSCKASGYTFTNFGMNWVKQAPGKGFKWMAWINTTRYTGESYFADDFKGRFAFSVETSATTAYLQINNLKTEDTATYFCARGEIYYGYDGGFAYWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQIGGGGSGGGGSGGGGSQVQLVQSGGGVVQPGRSLRLSCASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSTTTPAPRPPTPAPTIASQPLSLRPEACRPAAGGAVHTRGLDFACDFWVLVVVGGVLACYSLLVTVAFIIFWV

SEQ ID NO: 14 - DNA, 2535 bp, Artificial Sequence
  Synthetic Sequence
atggccctgcccgtgaccgccctgctgctgcccctggccctgctgctgcacgccgccaggcccgacgtggtgatgacccagagccacaggttcatgagcaccagcgtgggcgacagggtgagcatcacctgcagggccagccaggacgtgaacaccgccgtgagctggtaccagcagaagcccggccagagccccaagctgctgatcttcagcgccagctacaggtacaccggcgtgcccgacaggttcaccggcagcggcagcggcgccgacttcaccctgaccatcagcagcgtgcaggccgaggacctggccgtgtactactgccagcagcactacagcaccccctggaccttcggcggcggcaccaagctggacatcaagggcggcggcggcagcggcggcggcggcagcggcggcggcggcagccagatccagctggtgcagagcggccccgacctgaagaagcccggcgagaccgtgaagctgagctgcaaggccagcggctacaccttcaccaacttcggcatgaactgggtgaagcaggcccccggcaagggcttcaagtggatggcctggatcaacaccaccaggtacaccggcgagagctacttcgccgacgacttcaagggcaggttcgccttcagcgtggagaccagcgccaccaccgcctacctgcagatcaacaacctgaagaccgaggacaccgccacctacttctgcgccaggggcgagatctactacggctacgacggcggcttcgcctactggggccagggcaccctggtgaccgtgagcgccaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagccccagaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggggcagcggcgccaccaacttcagcctgctgaagcaggccggcgacgtggaggagaaccccggccccatggccctgcccgtgaccgccctgctgctgcccctggccctgctgctgcacgccgccaggcccgacatccagatgacccagagccccagcagcctgagcgccagcgtgggcgacagggtgaccatcacctgcagcgccagcagcagcgtgagctacatgaactggtaccagcagacccccggcaaggcccccaagaggtggatctacgacaccagcaagctggccagcggcgtgcccagcaggttcagcggcagcggcagcggcaccgactacaccttcaccatcagcagcctgcagcccgaggacatcgccacctactactgccagcagtggagcagcaaccccttcaccttcggccagggcaccaagctgcagatcggcggcggcggcagcggcggcggcggcagcggcggcggcggcagccaggtgcagctggtgcagagcggcggcggcgtggtgcagcccggcaggagcctgaggctgagctgcgccagcggctacaccttcaccaggtacaccatgcactgggtgaggcaggcccccggcaagggcctggagtggatcggctacatcaaccccagcaggggctacaccaactacaaccagaaggtgaaggacaggttcaccatcagcagggacaacagcaagaacaccgccttcctgcagatggacagcctgaggcccgaggacaccggcgtgtacttctgcgccaggtactacgacgaccactactgcctggactactggggccagggcacccccgtgaccgtgagcagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacttctgggtgctggtggtggtgggcggcgtgctggcctgctacagcctgctggtgaccgtggccttcatcatcttctgggtgtaa

SEQ ID NO: 15 - protein, 841 aa, Artificial Sequence
  Synthetic Sequence
MALPVTALLLPLALLLHAARPQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQIGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSTTTPAPRPPTPAPTIASQPLSLRPEACRPAAGGAVHTRGLDFACDFWVLVVVGGVLACYSLLVTVAFIIFWV

SEQ ID NO: 16 - DNA, 2526 bp, Artificial Sequence
  Synthetic Sequence
atggccctgcccgtgaccgccctgctgctgcccctggccctgctgctgcacgccgccaggccccagatcgtgctgagccagagccccgccatcctgagcgccagccccggcgagaaggtgaccatgacctgcagggccagcagcagcgtgagctacatccactggttccagcagaagcccggcagcagccccaagccctggatctacgccaccagcaacctggccagcggcgtgcccgtgaggttcagcggcagcggcagcggcaccagctacagcctgaccatcagcagggtggaggccgaggacgccgccacctactactgccagcagtggaccagcaacccccccaccttcggcggcggcaccaagctggagatcaagggcggcggcggcagcggcggcggcggcagcggcggcggcggcagccaggtgcagctgcagcagcccggcgccgagctggtgaagcccggcgccagcgtgaagatgagctgcaaggccagcggctacaccttcaccagctacaacatgcactgggtgaagcagacccccggcaggggcctggagtggatcggcgccatctaccccggcaacggcgacaccagctacaaccagaagttcaagggcaaggccaccctgaccgccgacaagagcagcagcaccgcctacatgcagctgagcagcctgaccagcgaggacagcgccgtgtactactgcgccaggagcacctactacggcggcgactggtacttcaacgtgtggggcgccggcaccaccgtgaccgtgagcgccaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagccccagaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggggcagcggcgccaccaacttcagcctgctgaagcaggccggcgacgtggaggagaaccccggccccatggccctgcccgtgaccgccctgctgctgcccctggccctgctgctgcacgccgccaggcccgacatccagatgacccagagccccagcagcctgagcgccagcgtgggcgacagggtgaccatcacctgcagcgccagcagcagcgtgagctacatgaactggtaccagcagacccccggcaaggcccccaagaggtggatctacgacaccagcaagctggccagcggcgtgcccagcaggttcagcggcagcggcagcggcaccgactacaccttcaccatcagcagcctgcagcccgaggacatcgccacctactactgccagcagtggagcagcaaccccttcaccttcggccagggcaccaagctgcagatcggcggcggcggcagcggcggcggcggcagcggcggcggcggcagccaggtgcagctggtgcagagcggcggcggcgtggtgcagcccggcaggagcctgaggctgagctgcaaggccagcggctacaccttcaccaggtacaccatgcactgggtgaggcaggcccccggcaagggcctggagtggatcggctacatcaaccccagcaggggctacaccaactacaaccagaaggtgaaggacaggttcaccatcagcagggacaacagcaagaacaccgccttcctgcagatggacagcctgaggcccgaggacaccggcgtgtacttctgcgccaggtactacgacgaccactactgcctggactactggggccagggcacccccgtgaccgtgagcagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacttctgggtgctggtggtggtgggcggcgtgctggcctgctacagcctgctggtgaccgtggccttcatcatcttctgggtgtaa

SEQ ID NO: 17 - protein, 817 aa, Artificial Sequence
  Synthetic Sequence
MALPVTALLLPLALLLHAARPDIQLTQSPAILSASPGEKVTMTCRASSSLSFMHWYQQKPGSSPKPWIYATSNLASGVPARFSGSGSGTSYSLTISTVEAEDAASYFCHQWSSNPLTFGAGTKLEISSGGGGSGGGGSGDVMGVDSGGGLVQPGGSRKLSCAAPGFTFSSFGMHWVRQAPEKGLEWVAYISSPSSTLHYADRVKGRFTISRDNPKNTLFLQMKLPSLCYGLLGPRDHVHTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQIGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSTTTPAPRPPTPAPTIASQPLSLRPEACRPAAGGAVHTRGLDFACDFWVLVVVGGVLACYSLLVTVAFIIFWV

SEQ ID NO: 18 - DNA, 2454 bp, Artificial Sequence
  Synthetic Sequence
atggccctgcccgtgaccgccctgctgctgcccctggccctgctgctgcacgccgccaggcccgacatccagctgacccagagccccgccatcctgagcgccagccccggcgagaaggtgaccatgacctgcagggccagcagcagcctgagcttcatgcactggtaccagcagaagcccggcagcagccccaagccctggatctacgccaccagcaacctggccagcggcgtgcccgccaggttcagcggcagcggcagcggcaccagctacagcctgaccatcagcaccgtggaggccgaggacgccgccagctacttctgccaccagtggagcagcaaccccctgaccttcggcgccggcaccaagctggagatcagcagcggcggcggcggcagcggcggcggcggcagcggcgacgtgatgggcgtggacagcggcggcggcctggtgcagcccggcggcagcaggaagctgagctgcgccgcccccggcttcaccttcagcagcttcggcatgcactgggtgaggcaggcccccgagaagggcctggagtgggtggcctacatcagcagccccagcagcaccctgcactacgccgacagggtgaagggcaggttcaccatcagcagggacaaccccaagaacaccctgttcctgcagatgaagctgcccagcctgtgctacggcctgctgggccccagggaccacgtgcacaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagccccagaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggggcagcggcgccaccaacttcagcctgctgaagcaggccggcgacgtggaggagaaccccggccccatggccctgcccgtgaccgccctgctgctgcccctggccctgctgctgcacgccgccaggcccgacatccagatgacccagagccccagcagcctgagcgccagcgtgggcgacagggtgaccatcacctgcagcgccagcagcagcgtgagctacatgaactggtaccagcagacccccggcaaggcccccaagaggtggatctacgacaccagcaagctggccagcggcgtgcccagcaggttcagcggcagcggcagcggcaccgactacaccttcaccatcagcagcctgcagcccgaggacatcgccacctactactgccagcagtggagcagcaaccccttcaccttcggccagggcaccaagctgcagatcggcggcggcggcagcggcggcggcggcagcggcggcggcggcagccaggtgcagctggtgcagagcggcggcggcgtggtgcagcccggcaggagcctgaggctgagctgcaaggccagcggctacaccttcaccaggtacaccatgcactgggtgaggcaggcccccggcaagggcctggagtggatcggctacatcaaccccagcaggggctacaccaactacaaccagaaggtgaaggacaggttcaccatcagcagggacaacagcaagaacaccgccttcctgcagatggacagcctgaggcccgaggacaccggcgtgtacttctgcgccaggtactacgacgaccactactgcctggactactggggccagggcacccccgtgaccgtgagcagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacttctgggtgctggtggtggtgggcggcgtgctggcctgctacagcctgctggtgaccgtggccttcatcatcttctgggtgtaa

SEQ ID NO: 19 - protein, 846 aa, Artificial Sequence
  Synthetic Sequence
MALPVTALLLPLALLLHAARPDIQMTQSPSSLSASVGDRVTITCRSSQSIVHSVGNTFLEWYQQKPGKAPKLLIYKVSNRFSGVPSRFSGSGSGTDFTLTISSLQPEDFATYYCFQGSQFPYTFGQGTKVEIKGGGGSGGGGSGGGGSEVQLVESGGGLVQPGGSLRLSCAASGYEFSRSWMNWVRQAPGKGLEWVGRIYPGDGDTNYSGKFKGRFTISADTSKNTAYLQMNSLRAEDTAVYYCARDGSSWDWYFDVWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQIGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSTTTPAPRPPTPAPTIASQPLSLRPEACRPAAGGAVHTRGLDFACDFWVLVVVGGVLACYSLLVTVAFIIFWV

SEQ ID NO: 20 - DNA, 2541 bp, Artificial Sequence
  Synthetic Sequence
atggccctgcccgtgaccgccctgctgctgcccctggccctgctgctgcacgccgccaggcccgacatccagatgacccagagccccagcagcctgagcgccagcgtgggcgacagggtgaccatcacctgcaggagcagccagagcatcgtgcacagcgtgggcaacaccttcctggagtggtaccagcagaagcccggcaaggcccccaagctgctgatctacaaggtgagcaacaggttcagcggcgtgcccagcaggttcagcggcagcggcagcggcaccgacttcaccctgaccatcagcagcctgcagcccgaggacttcgccacctactactgcttccagggcagccagttcccctacaccttcggccagggcaccaaggtggagatcaagggcggcggcggcagcggcggcggcggcagcggcggcggcggcagcgaggtgcagctggtggagagcggcggcggcctggtgcagcccggcggcagcctgaggctgagctgcgccgccagcggctacgagttcagcaggagctggatgaactgggtgaggcaggcccccggcaagggcctggagtgggtgggcaggatctaccccggcgacggcgacaccaactacagcggcaagttcaagggcaggttcaccatcagcgccgacaccagcaagaacaccgcctacctgcagatgaacagcctgagggccgaggacaccgccgtgtactactgcgccagggacggcagcagctgggactggtacttcgacgtgtggggccagggcaccctggtgaccgtgagcagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagccccagaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggggcagcggcgccaccaacttcagcctgctgaagcaggccggcgacgtggaggagaaccccggccccatggccctgcccgtgaccgccctgctgctgcccctggccctgctgctgcacgccgccaggcccgacatccagatgacccagagccccagcagcctgagcgccagcgtgggcgacagggtgaccatcacctgcagcgccagcagcagcgtgagctacatgaactggtaccagcagacccccggcaaggcccccaagaggtggatctacgacaccagcaagctggccagcggcgtgcccagcaggttcagcggcagcggcagcggcaccgactacaccttcaccatcagcagcctgcagcccgaggacatcgccacctactactgccagcagtggagcagcaaccccttcaccttcggccagggcaccaagctgcagatcggcggcggcggcagcggcggcggcggcagcggcggcggcggcagccaggtgcagctggtgcagagcggcggcggcgtggtgcagcccggcaggagcctgaggctgagctgcaaggccagcggctacaccttcaccaggtacaccatgcactgggtgaggcaggcccccggcaagggcctggagtggatcggctacatcaaccccagcaggggctacaccaactacaaccagaaggtgaaggacaggttcaccatcagcagggacaacagcaagaacaccgccttcctgcagatggacagcctgaggcccgaggacaccggcgtgtacttctgcgccaggtactacgacgaccactactgcctggactactggggccagggcacccccgtgaccgtgagcagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacttctgggtgctggtggtggtgggcggcgtgctggcctgctacagcctgctggtgaccgtggccttcatcatcttctgggtgtaa

SEQ ID NO: 21 - protein, 848 aa, Artificial Sequence
  Synthetic Sequence
MALPVTALLLPLALLLHAARPEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQIGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSTTTPAPRPPTPAPTIASQPLSLRPEACRPAAGGAVHTRGLDFACDFWVLVVVGGVLACYSLLVTVAFIIFWV

SEQ ID NO: 22 - DNA, 2547 bp, Artificial Sequence
  Synthetic Sequence
atggccctgcccgtgaccgccctgctgctgcccctggccctgctgctgcacgccgccaggcccgaggtgcagctgcagcagagcggccccgagctgatcaagcccggcgccagcgtgaagatgagctgcaaggccagcggctacaccttcaccagctacgtgatgcactgggtgaagcagaagcccggccagggcctggagtggatcggctacatcaacccctacaacgacggcaccaagtacaacgagaagttcaagggcaaggccaccctgaccagcgacaagagcagcagcaccgcctacatggagctgagcagcctgaccagcgaggacagcgccgtgtactactgcgccaggggcacctactactacggcagcagggtgttcgactactggggccagggcaccaccctgaccgtgagcagcggcggcggcggcagcggcggcggcggcagcggcggcggcggcagcgacatcgtgatgacccaggccgcccccagcatccccgtgacccccggcgagagcgtgagcatcagctgcaggagcagcaagagcctgctgaacagcaacggcaacacctacctgtactggttcctgcagaggcccggccagagcccccagctgctgatctacaggatgagcaacctggccagcggcgtgcccgacaggttcagcggcagcggcagcggcaccgccttcaccctgaggatcagcagggtggaggccgaggacgtgggcgtgtactactgcatgcagcacctggagtaccccttcaccttcggcgccggcaccaagctggagctgaagaggaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagccccagaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggggcagcggcgccaccaacttcagcctgctgaagcaggccggcgacgtggaggagaaccccggccccatggccctgcccgtgaccgccctgctgctgcccctggccctgctgctgcacgccgccaggcccgacatccagatgacccagagccccagcagcctgagcgccagcgtgggcgacagggtgaccatcacctgcagcgccagcagcagcgtgagctacatgaactggtaccagcagacccccggcaaggcccccaagaggtggatctacgacaccagcaagctggccagcggcgtgcccagcaggttcagcggcagcggcagcggcaccgactacaccttcaccatcagcagcctgcagcccgaggacatcgccacctactactgccagcagtggagcagcaaccccttcaccttcggccagggcaccaagctgcagatcggcggcggcggcagcggcggcggcggcagcggcggcggcggcagccaggtgcagctggtgcagagcggcggcggcgtggtgcagcccggcaggagcctgaggctgagctgcaaggccagcggctacaccttcaccaggtacaccatgcactgggtgaggcaggcccccggcaagggcctggagtggatcggctacatcaaccccagcaggggctacaccaactacaaccagaaggtgaaggacaggttcaccatcagcagggacaacagcaagaacaccgccttcctgcagatggacagcctgaggcccgaggacaccggcgtgtacttctgcgccaggtactacgacgaccactactgcctggactactggggccagggcacccccgtgaccgtgagcagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacttctgggtgctggtggtggtgggcggcgtgctggcctgctacagcctgctggtgaccgtggccttcatcatcttctgggtgtaa

SEQ ID NO: 23 - DNA, 454 bp, Artificial Sequence
  Synthetic Sequence
gcgatcgcatggccttaccagtgaccgccttgctcctgccgctggccttgctgctccacgccgccaggccggactacaaggaccacgacggcgactacaaggaccacgacatcgactacaaggacgacgacgacaagagccccctgttccccggccccagcaagcccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcggcagcggcgagggcaggggcagcctgctgacctgcggcgacgtggaggagaaccccggcccctctagataaagcgtttaaac

SEQ ID NO: 24 - protein, 144 aa, Artificial Sequence
  Synthetic Sequence
MALPVTALLLPLALLLHAARPDYKDHDGDYKDHDIDYKDDDDKSPLFPGPSKPFWVLVVVGGVLACYSLLVTVAFIIFWVRSKRSRLLHSDYMNMTPRRPGPTRKHYQPYAPPRDFAAYRSGSGEGRGSLLTCGDVEENPGPSR

SEQ ID NO: 25 - DNA, 457 bp, Artificial Sequence
  Synthetic Sequence
gcgatcgcatggccttaccagtgaccgccttgctcctgccgctggccttgctgctccacgccgccaggccggactacaaggaccacgacggcgactacaaggaccacgacatcgactacaaggacgacgacgacaagcccgccagggagcccggccacagcccccagatcatcagcttcttcctggccctgaccagcaccgccctgctgttcctgctgttcttcctgaccctgaggttcagcgtggtgaagaggggcaggaagaagctgctgtacatcttcaagcagcccttcatgaggcccgtgcagaccacccaggaggaggacggctgcagctgcaggttccccgaggaggaggagggcggctgcgagctgggcagcggcgagggcaggggcagcctgctgacctgcggcgacgtggaggagaaccccggcccctctagataaagcgtttaaac

SEQ ID NO: 26 - protein, 145 aa, Artificial Sequence
  Synthetic Sequence
MALPVTALLLPLALLLHAARPDYKDHDGDYKDHDIDYKDDDDKPAREPGHSPQIISFFLALTSTALLFLLFFLTLRFSVVKRGRKKLLYIFKQPFMRPVQTTQEEDGCSCRFPEEEEGGCELGSGEGRGSLLTCGDVEENPGPSR

SEQ ID NO: 27 - protein, 24 aa, Artificial Sequence
  synthetic sequence
NSELLSLINDMPITNDQKKLMSNN

SEQ ID NO: 28 - protein, 15 aa, Artificial Sequence
  synthetic sequence
GLNDIFEAQKIEWHE

SEQ ID NO: 29 - protein, 25 aa, Artificial Sequence
  synthetic sequence
KRRWKKNFIAVSAANRFKKISSSGA

SEQ ID NO: 30 - protein, 6 aa, Artificial Sequence
  synthetic sequence
EEEEEE

SEQ ID NO: 31 - protein, 13 aa, Artificial Sequence
  synthetic sequence
GAPVPYPDPLEPR

SEQ ID NO: 32 - protein, 8 aa, Artificial Sequence
  synthetic sequence
DYKDDDDK

SEQ ID NO: 33 - protein, 9 aa, Artificial Sequence
  synthetic sequence
YPYDVPDYA

SEQ ID NO: 34 - protein, 6 aa, Artificial Sequence
  synthetic sequence
HHHHHH

SEQ ID NO: 35 - protein, 10 aa, Artificial Sequence
  synthetic sequence
EQKLISEEDL

SEQ ID NO: 36 - protein, 18 aa, Artificial Sequence
  synthetic sequence
TKENPRSNQEESYDDNES

SEQ ID NO: 37 - protein, 15 aa, Artificial Sequence
  synthetic sequence
KETAAAKFERQHMDS

SEQ ID NO: 38 - protein, 38 aa, Artificial Sequence
  synthetic sequence
MDEKTTGWRGGHVVEGLAGELEQLRARLEHHPQGQREP

SEQ ID NO: 39 - protein, 13 aa, Artificial Sequence
  synthetic sequence
SLAELLNAGLGGS

SEQ ID NO: 40 - protein, 8 aa, Artificial Sequence
  synthetic sequence
TQDPSRVG

SEQ ID NO: 41 - protein, 8 aa, Artificial Sequence
  synthetic sequence
WSHPQFEK

SEQ ID NO: 42 - protein, 6 aa, Artificial Sequence
  synthetic sequence
CCPGCC

SEQ ID NO: 43 - protein, 14 aa, Artificial Sequence
  synthetic sequence
GKPIPNPLLGLDST

SEQ ID NO: 44 - protein, 11 aa, Artificial Sequence
  synthetic sequence
YTDIEMNRLGK

SEQ ID NO: 45 - protein, 8 aa, Artificial Sequence
  synthetic sequence
DLYDDDDK

SEQ ID NO: 46 - protein, 4 aa, Artificial Sequence
  synthetic sequence
KDEL